(19) United States
(12) Patent Application Publication
(10) Pub. No.: US 2025/0179155 A1
(43) Pub. Date: Jun. 5, 2025

(54) NANOBODIES FOR TREATING C. DIFFICILE INFECTION

(71) Applicants: VANDERBILT UNIVERSITY, Nashville, TN (US); The United States Government as Represented by the Department of Veterans Affairs, Washington, DC (US)

(72) Inventors: D. Borden Lacy, Nashville, TN (US); Benjamin W. Spiller, Nashville, TN (US)

(21) Appl. No.: 18/738,660

(22) Filed: Jun. 10, 2024

Related U.S. Application Data
(60) Provisional application No. 63/509,666, filed on Jun. 22, 2023.

(51) Int. Cl. C07K 16/12; A61P 31/04; G01N 33/569

(52) U.S. Cl. CPC C07K 16/1282; A61P 31/04; G01N 33/56911; C07K 2317/569; G01N 2333/33; G01N 2469/10

(57) ABSTRACT

C. difficile is a leading cause of antibiotic-associated diarrhea and nosocomial infection in the United States. The symptoms of C. difficile infection (CDI) are associated with the production of two homologous protein toxins, TcdA and TcdB, and the toxins are considered bone fide targets for clinical diagnosis as well as the development of novel prevention and therapeutic strategies. Disclosed herein are nanobodies that bind specific structural and functional domains of TcdA and TcdB.

### CROSS-REFERENCE TO RELATED APPLICATIONS

This application claims benefit of U.S. Provisional Application No. 63/509,666, filed Jun. 22, 2023, which is hereby incorporated herein by reference in its entirety.

### STATEMENT OF GOVERNMENT INTEREST

This invention was made with Government Support under Grant No. A1957555 awarded by the National Institutes of Health and BX002943 from the Department of Veterans Affairs. The Government has certain rights in the invention.

### SEQUENCE LISTING

This application contains a sequence listing filed in ST.26 format entitled “222230-1330 Sequence Listing” created on Jun. 10, 2024, and having 212,264 bytes. The content of the sequence listing is incorporated herein in its entirety.

### BACKGROUND OF THE INVENTION

Clostridioides difficile is the causative agent of C. difficile infection (CDI), a disease with symptoms ranging from mild diarrhea to more life-threatening conditions such as pseudomembranous colitis, and toxic megacolon. C. difficile has been classified as a “threat-level urgent” pathogen by the US Centers for Disease Control due to high levels of morbidity and mortality. Treatment options are limited, and vaccine efforts have not succeeded, motivating on-going research into novel prevention and therapeutic strategies.

Symptoms are associated with the production of up to three toxins, TcdA, TcdB, and CDT. TodA and TcdB are considered the main virulence factors and are large (308 and 270 kDa, respectively) glucosylating toxins which inhibit Rho-family GTPases. Multiple large scale vaccine trials focused on the use of toxoid antigens have shown promise in pre-clinical models but have failed to meet primary clinical endpoints in people. Among multiple avenues for optimization, there is a need to better understand the toxin sequences and structures that promote broadly neutralizing antibody responses. This is especially true for TcdB, as it is now appreciated that TcdB sequences can vary, with 5 or more sub-types (TcdB1-B5) prevalent among circulating clinical strains.

TcdA and TcdB share approximately 47% sequence identity and have four domains: the N-terminal glucosyltransferase domain (GTD), the autoprotease domain (APD), the delivery domain (DD), and the combined repetitive oligopeptides (CROPs) domain. TcdA and TcdB intoxicate cells via a multistep process: receptor binding and endocytosis, pH-dependent pore formation, translocation of the GTD and APD across the endosomal membrane, autoprocessing and GTD release, and finally, GTD-mediated glucosylation of host GTPases. In cell culture models, the glucosyltransferase activity causes cell rounding, a loss of tight junction formation, and apoptotic cell death, and studies performed in small animal models of infection point to a clear role for the glucosyltransferase activity in pathogenesis.

TcdA and TcdB can bind to multiple receptors, and not all receptor binding sites have been defined. Historically, receptor binding has been associated with the C-terminal CROPS domain. The TcdA CROPS has 7 repetitive sequence blocks (R1-R7) while the TcdB CROPS has 4 (R1-R4), and this repetition has been hypothesized to contribute to immunodominance. The sequence blocks engage glycans with low affinity, but these could promote high avidity interactions if multiple glycans are engaged simultaneously. More recent studies have identified receptor interactions outside of the CROPS domain. For example, TcdA can engage sulfated glycosaminoglycans in a CROPS-independent interaction with the sulfate group. TcdB has been reported to bind four protein receptors classes: chondroitin sulfate proteoglycan 4 (CSPG4); Frizzled (FZD) 1, FZD2, and FZD7; Nectin3; and TFP1. The interactions can vary with TcdB subtype, but arguably all bind in a CROPS-independent interaction. The interaction with CSPG4 occurs at the ‘hinge’ where the CROPS moves relative to the other toxin domains and involves some of the N-terminal residues of the CROPS. Similar to the variation in receptor binding with TcdB subtype, the neutralization efficacy of different monoclonal antibodies can differ depending on the TcdB subtype.

In addition to the need to define broadly neutralizing epitopes, there is also a need to assay and quantify the concentrations of TcdA and TcdB in an animal model of infection. While ELISAs are commercially available for the detection of TcdA and TcdB in human stool and are considered a key component of CDI diagnostics in many clinical microbiology laboratories, they have limited sensitivity and are considered non-quantitative for research purposes. Many labs make use of Vero cell rounding assays, and a proprietary reagent that neutralizes TcdA/TcdB induced rounding, but the reagent cannot differentiate between TcdA and TcdB. Some labs have access to a real time cellular impedance assay, which is sensitive and quantitative but is expensive and not readily able to differentiate between TcdA and TcdB.

### SUMMARY OF THE INVENTION

C. difficile is a leading cause of antibiotic-associated diarrhea and nosocomial infection in the United States. The symptoms of C. difficile infection (CDI) are associated with the production of two homologous protein toxins, TcdA and TcdB, and the toxins are considered bone fide targets for clinical diagnosis as well as the development of novel prevention and therapeutic strategies. While there are extensive studies that document these efforts, there are several gaps in knowledge that could benefit from the creation of new research tools. It was discovered that while TcdA sequences are stable, TcdB sequences can vary across the span of circulating clinical isolates. An understanding of the TcdA and TcdB epitopes that drive broadly neutralizing antibody responses could advance the effort to identify safe and effective toxin protein chimeras and fragments for vaccine development. Further, an understanding of how TcdA and TcdB expression changes in vivo, can guide research into how host and microbiome-focused interventions affect the virulence potential of C. difficile. To address these gaps, a panel of alpaca-derived nanobodies were developed that bind specific structural and functional domains of TcdA and TcdB. In testing for neutralization, it was noted that many of the potent neutralizers of TcdA bind epitopes within the delivery domain, a finding that could reflect roles of the delivery domain in receptor binding and/or the conserved role of pore-formation in the delivery of the toxin enzyme domains to the cytosol. In contrast, neutralizing epitopes for TodB were found in multiple domains. The nanobodies were also used for the creation of sandwich ELISA assays. These assays allow for quantitation of TcdA and/or TcdB in vitro and in the cecal and fecal contents of infected mice. As disclosed herein, these reagents and assays are tools that can allow researchers to monitor the dynamics of TcdA and TcdB production over time, and the impact of various experimental interventions on toxin production in vivo.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows a process for developing nanobodies.

FIGS. 2A to 2K show negative stain EM of TcdA- and TcdB-nanobody (Nb) complexes. FIG. 2A shows TcdA₁₋₁₈₃₂ domain organization used for negative stain. GTD (glucosyltransferase domain); APD (autoprotease domain); DD (delivery domain). 2D class averages determined from TcdA₁₋₁₈₃₂ with 2-fold molar excess of nanobody. White arrow indicates location of Nb. FIGS. 2B to 2F show A1D1 (FIG. 2B), A2B5 (FIG. 2C), A1D8 (FIG. 2D), A2H9 (FIG. 2E), or A1C3 (FIG. 2F). Below are space filling structures using domain colors found in FIG. 2A (PDB: 4R04) with the Nb binding locations from the 2D averages circled. FIG. 2G shows TcdB₁₋₁₈₁₀ domain organization. 2D class averages determined from TcdB₁₋₁₈₁₀ with 2-fold molar excess of Nb. White arrow indicates location of Nb. FIGS. 2H to 2K show B0E2 (FIG. 2H), B0D11 (FIG. 2I), B0D10 (FIG. 2J), or B2C11 (FIG. 2K). Below are space filling structures (PDB: 6OQ5) with the Nb binding locations from the 2D averages circled. Scale bar: 100 Å.

FIGS. 3A to 3P show development of an anti-TcdA nanobody (Nb) based sandwich ELISA. FIG. 3A shows detection of purified, recombinant TcdA (rTcdA) by sandwich ELISA using capture Nb A2B10 (anti-CROPs) and detection Nb A1A6 (anti-GTD) using two-fold serial dilutions of TcdA. FIG. 3B shows a standard curve using two fold serial dilutions of rTcdA in either PBST+BSA or filtered supernatant of M7404 tcdA::ermB todB::ermB. FIG. 3C shows an ELISA measuring TcdA in filtered supernatant of M7404 tcdA::ermB (black), M7404 tcdB::ermB, or M7404 todA::ermB tcdB::ermB (aqua). FIG. 3D shows use of the A2B10/A1A6 sandwich ELISA to quantify TcdA in C. difficile filtered supernatant of strains M7404, R20291 (light purple), and VPI10463. FIG. 3E is a standard curve for rTcdA in feces using the A2B10/A1A6 Nb pair and two-fold serial dilutions. Limit of detection (LOD) noted by dashed line and was determined in roughly 50 mg/ml of feces. FIG. 3F is a standard curve for rTcdA in cecal content using the A2B10/A1A6 Nb pair and two-fold serial dilutions of TcdA. LOD noted by dashed line and was determined in roughly 500 mg/ml of cecal content. FIG. 3G shows evaluation of two anti-delivery domain (DD) Nbs (A1D8 and A1C3) in the sandwich ELISA assay using capture Nb A1D8 and detection Nb A1C3. A1D8 was used to coat the plate, followed by two-fold serial dilutions of rTcdA except where noted. FIG. 3H is a standard curve using two fold serial dilutions of rTcdA in either PBST+BSA or filtered supernatant of M7404 tcdA::ermB tcdB::ermB. FIG. 3I shows an ELISA measuring TcdA in filtered supernatant of M7404 tcdA::ermB tcdB::ermB (black), M7404 tcdA::ermB M7404, M7404 tcdB::ermB. FIG. 3J shows A1D8/A1C3 ELISAs recognizes TcdA in C. difficile filtered supernatant of strains M7404, R20291, and VPI10463. FIG. 3K is a standard curve for rTcdA in feces using A1D8/A1C3 Nb pair and two-fold serial dilutions of TcdA. LOD noted by dashed line and was determined in roughly 50 mg/ml of feces. Inset shows linear range down to 0.075 ng/ml of TcdA. FIG. 3L is a standard curve for rTcdA in cecal content using A1D8/A1C3 Nb pair and two-fold serial dilutions of TcdA. LOD noted by dashed line and was determined in roughly 500 mg/ml of cecal content. FIG. 3M shows evaluation of two anti-delivery domain (A2H9 and A1C3) in the sandwich ELISA assay using capture Nb A2H9 and detection Nb A1C3. A2H9 was used to coat the plate, followed by two-fold serial dilutions of rTcdA except where noted. FIG. 3N shows evaluating two DD Nb ELISA's for detecting TcdA in the feces of Clostridioides difficile infected mice using a sandwich ELISA. ELISAs were performed using Nb combinations A1D8/A1C3 and A2H9/A1C3 in the feces of mice infected with C. difficile TcdBGTX two days post infection. FIG. 3O shows use of the fecal standard curves from FIGS. 3E and 3K. ELISAs were performed using Nb combinations A2B10/A1A6 and A1D8/A1C3 on feces from mice infected with C. difficile TcdBGTX two days post infection. Filled circles represent samples that are below the LOD (dashed line). FIG. 3P shows use of the cecal content standard curves from FIGS. 3F and 3L. ELISAs were performed using Nb combinations A2B10/A1A6 and A1D8/A1C3 in the cecal contents of mice infected with C. difficile TcdBGTX two days post infection. Filled circles represent samples that are below the LOD (dashed line). All ELISAs were performed in biological triplicate and error bars represent standard error of the mean (SEM).

FIGS. 4A to 4L show development of an anti-TcdB nanobody (Nb) based sandwich ELISA. FIG. 4A shows detection of purified, recombinant TcdB from C. difficile str VPI10463 or R20291 (rTcdB_{VPI} or rTcdB027, respectively) by sandwich ELISA using capture Nb B2C11 (anti-GTD) and detection Nb B0E2 (anti-DD) using 1 nM TcdB. FIG. 4B is a standard curve using two fold serial dilutions of rTcdB_{VPI} in either PBST+BSA or filtered supernatant of M7404 tcdA::ermB tcdB::ermB. FIG. 4C shows an ELISA measuring TcdB in filtered supernatant of M7404 tcdA::ermB, M7404 tcdB::ermB, or M7404 tcdA::ermB tcdB::ermB. FIG. 4D shows use of the B2C11/B0E2 sandwich ELISA to quantify TcdB in C. difficile filtered supernatant of strains M7404, R20291, and VPI10463. FIG. 4E is a standard curve for rTcdB_{VPI} in feces using the B2C11/B0E2 Nb pair and two-fold serial dilutions. Limit of detection (LOD) noted by dashed line and was determined in roughly 50 mg/ml of feces. FIG. 4F is a standard curve for rTcdB_{VPI} in cecal content using the B2C11/B0E2 Nb pair and two-fold serial dilutions of TcdB. LOD noted by dashed line and was determined in roughly 500 mg/mL of cecal content. FIG. 4G shows evaluation of two anti-delivery domain (DD) Nbs (B0D10/B0E2) in the sandwich ELISA assay using capture Nb B0D10 and detection Nb B0E2. B0D10 was used to coat the plate, followed by two-fold serial dilutions of rTcdB_{VPI} or rTcdB₀₂₇ except where noted. FIG. 4H is a standard curve using two fold serial dilutions of rTcdB_{VPI} in either PBST+BSA or filtered supernatant of M7404 tcdA::ermB tcdB::ermB. Inset shows data points below 0.5 ng/ml TcdB. FIG. 4I is an ELISA measuring TcdB in filtered supernatant of M7404 tcdA::ermB tcdB::ermB, M7404 tcdA::ermB M7404, M7404 tcdB::ermB. FIG. 4J shows B0D10/B0E2 ELISAs recognizes TcdB in C. difficile filtered supernatant of strains M7404, R20291, and VPI10463. FIG. 4K is a standard curve for rTcdB_{VPI} in feces using B0D10/B0E2 Nb pair and two-fold serial dilutions of TcdB. FIG. 4L shows use of the fecal and cecal content standard curves from FIGS. 4E and 4F. ELISAs were performed using Nb combinations B2C11/B0E2 in the fecal and cecal contents of mice infected with C. difficile TcdB_{GTX} two days post infection. Filled circles represent samples that are below the LOD (dashed line). All ELISAs were performed in biological triplicate and error bars represent standard error of the mean (SEM).

FIGS. 5A and 5B are cladograms representing amino acid sequence analysis and domain specificity of TcdA (FIG. 5A) and TcdB (FIG. 5B) nanobodies. Highly identical clones from each panel were removed (TcdA cutoff 100% ID, TcdB cutoff 95% ID) for analysis. Total clone numbers for each group are in parentheses. Nanobodies used in the experiments in this study are highlighted in yellow. Domain specificity was determined by ELISA (circle GTD, square DD or APD-DD, star CROPs).

FIGS. 6A and 6B show data graphs of toxin neutralization assays for TcdA (FIG. 6A) or TcdB (FIG. 6B). Analysis was performed in GraphPad Prism by least squares fit of the model: log (agonist) vs. response—Variable slope (four parameters). Error bars represent standard error for triplicate experiments, where possible.

FIGS. 7A to 7D show screening nanobody pairs for anti-TcdA ELISAs. Detection of purified, recombinant TcdA by sandwich ELISA using: capture Nb A1C3 (anti-DD) and detection Nb A2B10 (anti-CROPs) (FIG. 7A), capture Nb A2B10 (anti-CROPs) and detection Nb A1C3 (anti-DD) (FIG. 7B), capture Nb A1D1 (anti-DD) and detection Nb A1C3 (anti-DD) (FIG. 7C), and capture Nb A2B5 (anti-DD) and detection Nb A1C3 (anti-DD) (FIG. 7D). Two-fold serial dilutions of rTcdA were used, except where noted. All ELISAs were performed in biological triplicate and error bars represent standard error of the mean (SEM).

FIGS. 8A to 8G show screening nanobody pairs for anti-TcdB ELISAs. Detection of purified, recombinant TcdB from C. difficile str VPI10463 or R20291 (rTcdBVPI or rTcdB027, respectively) by sandwich ELISA using: capture Nb B2C11 (anti-GTD) and detection Nb B1A11 (anti-CROPs) (FIG. 8A), capture Nb B2F11 (anti-CROPs) and detection Nb B0E2 (anti-DD) (FIG. 8B), capture Nb B2F11 (anti-CROPs) and detection Nb B1A11 (anti-CROPs) (FIG. 8C), capture Nb B2F11 (anti-CROPs) and detection Nb B0B11 (anti-GTD) (FIG. 8D), and capture Nb B2F11 (anti-CROPs) and detection Nb B1A11 (anti-CROPs) using 1 nM TcdB (FIG. 8E). Evaluation of anti-DD Nbs B0A12/B0E2 (FIG. 8F) and B1C11/B0E2 (FIG. 8G) in the sandwich ELISA assay using capture Nb B0A12 or B1C11, respectively, and detection Nb B0E2. B0A12 or B1C11 was used to coat the plate, followed by two-fold serial dilutions of rTcdBVPI or rTcdB027 except where noted. All ELISAs were performed in biological triplicate and error bars represent standard error of the mean (SEM).

FIGS. 9A and 9B show C. difficile R20291 TcdBGTX infection. Mice infected with 10⁴ spores of R20291 TcdB_{GTX} were monitored daily for colony forming units (CFU) (FIG. 9A) and weight loss (FIG. 9B). The mice were sacrificed after 2 days. Cecal contents and fecal contents were collected for TcdA/TcdB ELISAs.

FIG. 10 shows C. difficile R20291 CT::tcdA CT::tcdB infection. Mice infected with 10⁴ spores of R20291 CT::tcdA CT::tcdB were monitored daily for weight loss and sacrificed after 2 days. Cecal contents and fecal contents were collected for spike-in ELISAs.

FIG. 11 shows neutralization of TcdB1 on Vero cells.

FIG. 12 shows a mouse model of CDI.

FIG. 13 shows D1D2 treated mice lose less weight and regain to near pre-infection weight.

FIG. 14 shows no clear trends emerged for colony forming units (CFUs) up to day 6 post infection.

FIG. 15 shows both D1G1 and D1D2 protect against R20291 infection.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

As used herein, the terms “single domain antibody (VHH)” and “nanobodies” have the same meaning referring to a variable region of a heavy chain of an antibody, and construct a single domain antibody (VHH) consisting of only one heavy chain variable region. It is the smallest antigen-binding fragment with complete function. The nanobody may be produced by any means. For instance, the nanobody may be enzymatically or chemically produced by fragmentation of an intact antibody, it may be recombinantly produced from a gene encoding the partial antibody sequence, or it may be wholly or partially synthetically produced.

The term “antigen binding site” refers to a region of an antibody that specifically binds an epitope on an antigen.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “specifically binds”, as used herein refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “cell penetrating peptide”, “cell penetrating protein”, “CPP” and the like, as used herein, refer to a peptide or protein having an ability to pass through cellular membranes. In various embodiments, a CPP is conjugated to a nanobody disclosed herein to facilitate transport of the nanobody across the membrane. In some embodiments, a CPP is capable of being internalized into a cell and passing cellular membranes (including, inter alia, the outer “limiting” cell membrane (also commonly referred to as “plasma membrane”), endosomal membranes, and membranes of the endoplasmatic reticulum). In some embodiments, any possible mechanism of internalization is envisaged including both energy-dependent (i.e. active) transport mechanisms (e.g., endocytosis) and energy-independent (i.e. passive) transport mechanism (e.g., diffusion).

### Nanobodies

Disclosed are compositions and methods for endoscopic visualization of colorectal adenomas. Specifically, disclosed herein are TcdA-specific and TcdB-specific nanobodies capable of neutralizing the activity of the CDT toxin.

In some embodiments, the nanobody comprises a variable domain having CDR1, CDR2 and CDR3 sequences. For example, in some embodiments, the CDR1 sequence comprises the amino acid sequence GRIFSIKS (SEQ ID NO:1); CDR2 sequence of the variable domain comprises the amino acid sequence ITSGGST (SEQ ID NO:2); and the CDR3 sequence of the variable domain comprises the amino acid sequence RRVVVTPYPDEYEYDY (SEQ ID NO:3). In some embodiments, the nanobody has one or more conservative substitutions in SEQ ID NOs: 1, 2, and/or 3. For example, in some embodiments, the disclosed TcdA-specific nanobody has the amino acid sequence: QVQLQESGGGLVQPGGSLRLSCAASGRIFSIKSMGWYRQAPGKQRELVADITSGGSTNYADS VKGRFTISRDSAWNTVYLQMNSLKPEDTAVYYCRRVVVTPYPDEYEYDYWGQGTQVTVSS (SEQ ID NO:4, A1C3), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:4.

In some embodiments, the nanobody comprises a variable domain having CDR1, CDR2 and CDR3 sequences. For example, in some embodiments, the CDR1 sequence comprises the amino acid sequence GFIDDDYA (SEQ ID NO:5); CDR2 sequence of the variable domain comprises the amino acid sequence ISSSNGKI (SEQ ID NO:6); and the CDR3 sequence of the variable domain comprises the amino acid sequence AAETRGWSYCSGYGWSRYKY (SEQ ID NO:7). For example, in some embodiments the nanobody has one or more conservative substitutions in SEQ ID NOs: 5, 6, and/or 7. In some embodiments, the disclosed TcdA-specific nanobody has the amino acid sequence: QVQLQESGGGSVQAGGSLRLSCAASGFIDDDYAIGWFRQAPGKEREGISCISSSNGKIHYADS VKGRFTISEDIAKKTVYLQMNFLKPEDTAVYYCAAETRGWSYCSGYGWSRYKYWGQGTQVTV SS (SEQ ID NO:8, A1A6), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:8.

In some embodiments, the nanobody comprises a variable domain having CDR1, CDR2 and CDR3 sequences. For example, in some embodiments, the CDR1 sequence comprises the amino acid sequence GGTFSSYS (SEQ ID NO:9); CDR2 sequence of the variable domain comprises the amino acid sequence ITWRGIT (SEQ ID NO:10); and the CDR3 sequence of the variable domain comprises the amino acid sequence AARDRRAARIQEFDY (SEQ ID NO:11). In some embodiments, the nanobody has one or more conservative substitutions in SEQ ID NOs: 9, 10, and/or 11 For example, in some embodiments the disclosed TcdA-specific nanobody has the amino acid sequence: QVQLQESGGGLVQAGGSLRLSCAASGGTFSSYSVGWFRQAPGLEREFVGMITWRGITYFEDF VKDRFNISRDNAKNTVYLQMNSLKPEDTAVYSCAARDRRAARIQEFDYWGQGTQVTVSS (SEQ ID NO:12, A1C1), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:12.

In some embodiments, the nanobody comprises a variable domain having CDR1, CDR2 and CDR3 sequences. For example, in some embodiments, the CDR1 sequence comprises the amino acid sequence GRSFSINT (SEQ ID NO:13); CDR2 sequence of the variable domain comprises the amino acid sequence ITTGGNT (SEQ ID NO:14); and the CDR3 sequence of the variable domain comprises the amino acid sequence RTVVVTPYPDEFEYDY (SEQ ID NO:15). In some embodiments, the nanobody has one or more conservative substitutions in SEQ ID NOs: 13, 14, and/or 15. For example, in some embodiments the disclosed TcdA-specific nanobody has the amino acid sequence: QVQLQESGGGLVQPGGSLRLSCAASGRSFSINTMGWYRQAPGNKRDMVATITTGGNTNYAD SVKGRFTISRDNAKNTVYLQMNSLKPEDTAVYYCRTVVVTPYPDEFEYDYWGQGTQVTVSS (SEQ ID NO:16, A1C4), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:16.

In some embodiments, the nanobody comprises a variable domain having CDR1, CDR2 and CDR3 sequences. For example, in some embodiments, the CDR1 sequence comprises the amino acid sequence GRSFSINT (SEQ ID NO:17); CDR2 sequence of the variable domain comprises the amino acid sequence ITTGGNT (SEQ ID NO:18); and the CDR3 sequence of the variable domain comprises the amino acid sequence RTVVVTPYPDEFEYDY (SEQ ID NO:19). In some embodiments, the nanobody has one or more conservative substitutions in SEQ ID NOs: 17, 18, and/or 19. For example, in some embodiments the disclosed TcdA-specific nanobody has the amino acid sequence: QVQLQESGGGLVQAGDSLRLSCAASGRTFSRYAMGWFRQAPGKEREFVAAISWSGGTTYYA DSVKGRFTISRDNAKNTVYLQMNSLKPEDTAVYYCAAGLFRGDLTRFKLDEYDYRGQGTQVTV SS (SEQ ID NO:20, A1C11), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:20.

In some embodiments, the nanobody comprises a variable domain having CDR1, CDR2 and CDR3 sequences. For example, in some embodiments, the CDR1 sequence comprises the amino acid sequence GRTFSTHT (SEQ ID NO:21); CDR2 sequence of the variable domain comprises the amino acid sequence IRWSDGMT (SEQ ID NO:22); and the CDR3 sequence of the variable domain comprises the amino acid sequence GAGPTMYHPTY (SEQ ID NO:23). In some embodiments, the nanobody has one or more conservative substitutions in SEQ ID NOs: 21, 22, and/or 23 For example, in some embodiments the disclosed TcdA-specific nanobody has the amino acid sequence: QVQLQESGGGLVQAGGSLRLSCAASGRTFSTHTMAWFRQAPGKEREFVTGIRWSDGMTIYA DSVKGRFTISRDKATNTMYLEMNTLKPDDTAVYYCGAGPTMYHPTYWGQGTQVTVSS (SEQ ID NO:160, A1D1), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:160.

In some embodiments, the nanobody comprises a variable domain having CDR1, CDR2 and CDR3 sequences. For example, in some embodiments, the CDR1 sequence comprises the amino acid sequence GGTFSRYA (SEQ ID NO:24); CDR2 sequence of the variable domain comprises the amino acid sequence ISYSGATT (SEQ ID NO:25); and the CDR3 sequence of the variable domain comprises the amino acid sequence AAGFFRGDLTKFKLDEYDY (SEQ ID NO:26). In some embodiments, the nanobody has one or more conservative substitutions in SEQ ID NOs: 24, 25, and/or 26. For example, in some embodiments the disclosed TcdA-specific nanobody has the amino acid sequence: QVQLQESGGGLVQAGGSLRLSCVASGGTFSRYALGWFRQAPGKEREFVAAISYSGATTYYAD SVKGRFTISRDNAKNTVFLQMNSLKPEDTAVYYCAAGFFRGDLTKFKLDEYDYRGQGTQVTVS S (SEQ ID NO:27, A1D8), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:27.

In some embodiments, the nanobody comprises a variable domain having CDR1, CDR2 and CDR3 sequences. For example, in some embodiments, the CDR1 sequence comprises the amino acid sequence GRTFTTYN (SEQ ID NO:28); CDR2 sequence of the variable domain comprises the amino acid sequence ITGLTRHT (SEQ ID NO:29); and the CDR3 sequence of the variable domain comprises the amino acid sequence AVSSGGDLNERVNYEY (SEQ ID NO:30). In some embodiments, the nanobody has one or more conservative substitutions in SEQ ID NOs: 28, 29, and/or 30. For example, in some embodiments the disclosed TcdA-specific nanobody has the amino acid sequence: QVQLQESGGGLVQAGGSLRLSCTASGRTFTTYNWAWFCQAPGKEREFVAAITGLTRHTYYAD SVKGRFTISRDNAKNTVYLQMNSLKPEDTAVYFCAVSSGGDLNERVNYEYWGQGTQVTVSS (SEQ ID NO:31, A1F4), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:31.

In some embodiments, the nanobody comprises a variable domain having CDR1, CDR2 and CDR3 sequences. For example, in some embodiments, the CDR1 sequence comprises the amino acid sequence GRTLNSYA (SEQ ID NO:32); CDR2 sequence of the variable domain comprises the amino acid sequence ISRAGGMT (SEQ ID NO:33); and the CDR3 sequence of the variable domain comprises the amino acid sequence AASFALVDSAGAYDY (SEQ ID NO:34). In some embodiments, the nanobody has one or more conservative substitutions in SEQ ID NOs: 32, 33, and/or 34. For example, in some embodiments disclosed TcdA-specific nanobody has the amino acid sequence: QVQLQESGGGLVQAGGSLRLSCAVSGRTLNSYAMGWFRQALGKEREFVAGISRAGGMTRYT DSVKGRFTISRDDAKNTVYLQMNSLKPDDTAVYSCAASFALVDSAGAYDYWGQGTQVTVSS (SEQ ID NO:35, A1G4), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:35.

In some embodiments, the nanobody comprises a variable domain having CDR1, CDR2 and CDR3 sequences. For example, in some embodiments, the CDR1 sequence comprises the amino acid sequence GRAFSSYA (SEQ ID NO:36); CDR2 sequence of the variable domain comprises the amino acid sequence ISWSGGST (SEQ ID NO:37); and the CDR3 sequence of the variable domain comprises the amino acid sequence AADFSQPLLATVPDDYDY (SEQ ID NO:38). In some embodiments, the nanobody has one or more conservative substitutions in SEQ ID NOs: 36, 37, and/or 38. For example, in some embodiments the disclosed TcdA-specific nanobody has the amino acid sequence: QVQLQESGGGLVQPGGSLRLSCAASGRAFSSYAMGWFRQTPGKEREFVAVISWSGGSTYYA DSVKGRFTISRDNTKNMVYLQMMSLKPEDTAVYYCAADFSQPLLATVPDDYDYWGQGTQVTV SS (SEQ ID NO:39, A1G6), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:39.

In some embodiments, the nanobody comprises a variable domain having CDR1, CDR2 and CDR3 sequences. For example, in some embodiments, the CDR1 sequence comprises the amino acid sequence GGTFSKTS (SEQ ID NO:40); CDR2 sequence of the variable domain comprises the amino acid sequence ITWSGNT (SEQ ID NO:41); and the CDR3 sequence of the variable domain comprises the amino acid sequence AARERTAARIQEFDY (SEQ ID NO:42). In some embodiments, the nanobody has one or more conservative substitutions in SEQ ID NOs: 40, 41, and/or 42. For example, in some embodiments the disclosed TcdA-specific nanobody has the amino acid sequence: QVQLQESGGGLVQAGGSLRLSCAASGGTFSKTSVGWFRQAPGLEREFVALITWSGNTYFVDS VRERFAISRDNAENTVYLQMNSLEPEDTAVYYCAARERTAARIQEFDYWGQGTQVTVSS (SEQ ID NO:43, A1H1), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:43.

In some embodiments, the nanobody comprises a variable domain having CDR1, CDR2 and CDR3 sequences. For example, in some embodiments, the CDR1 sequence comprises the amino acid sequence GSIFSINA (SEQ ID NO:44); CDR2 sequence of the variable domain comprises the amino acid sequence ITSGGST (SEQ ID NO:45); and the CDR3 sequence of the variable domain comprises the amino acid sequence HVPWTDDFGWAVKDY (SEQ ID NO:46). In some embodiments, the nanobody has one or more conservative substitutions in SEQ ID NOs: 44, 45, and/or 46. For example, in some embodiments the disclosed TcdA-specific nanobody has the amino acid sequence: QVQLQESGGGLVQPGGSLRLSCAASGSIFSINAMGWYRQAPGKQRELVASITSGGSTNYADSV KGRFTISRDGAKNTVYLQMNSLKPEDTAVYYCHVPWTDDFGWAVKDYWGQGTQVTVSS (SEQ ID NO:47, A1H5), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:47.

In some embodiments, the nanobody comprises a variable domain having CDR1, CDR2 and CDR3 sequences. For example, in some embodiments, the CDR1 sequence comprises the amino acid sequence GRTFSRYE (SEQ ID NO:48); CDR2 sequence of the variable domain comprises the amino acid sequence INRLGRST (SEQ ID NO:49); and the CDR3 sequence of the variable domain comprises the amino acid sequence AAGVRLNLPQIPDVIDF (SEQ ID NO:50). In some embodiments, the nanobody has one or more conservative substitutions in SEQ ID NOs: 48, 49, and/or 50. For example, in some embodiments the disclosed TcdA-specific nanobody has the amino acid sequence: QVQLQESGGGLVQAGGSLRLSCADSGRTFSRYEMGWSRQAPGKEREFVAVINRLGRSTYYA DSVKGRFTISRDNAKNTVYLQMNSLKPEDTAVYYCAAGVRLNLPQIPDVIDFWGQGTQVTVSS (SEQ ID NO:51, A2A6), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:51.

In some embodiments, the nanobody comprises a variable domain having CDR1, CDR2 and CDR3 sequences. For example, in some embodiments, the CDR1 sequence comprises the amino acid sequence GGTFSSYS (SEQ ID NO:52); CDR2 sequence of the variable domain comprises the amino acid sequence ITWRNNT (SEQ ID NO:53); and the CDR3 sequence of the variable domain comprises the amino acid sequence AARERRVARIQEFDY (SEQ ID NO:54). In some embodiments, the nanobody has one or more conservative substitutions in SEQ ID NOs: 52, 53, and/or 54. For example, in some embodiments the disclosed TcdA-specific nanobody has the amino acid sequence: QVQLQESGGGLVQAGDSLRLSCTASGGTFSSYSIGWFRQAPGLEREFVALITWRNNTYLGDS VRERFAISRDNAKNTVYLQMNSLKPEDTAVYSCAARERRVARIQEFDYWGQGTQVTVSS (SEQ ID NO:55, A2A8), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:55.

In some embodiments, the nanobody comprises a variable domain having CDR1, CDR2 and CDR3 sequences. For example, in some embodiments, the CDR1 sequence comprises the amino acid sequence GRDFSSGA (SEQ ID NO:56); CDR2 sequence of the variable domain comprises the amino acid sequence VGWSGGLI (SEQ ID NO:57); and the CDR3 sequence of the variable domain comprises the amino acid sequence AVNSANSCAGYDCHDKPQTYNY (SEQ ID NO:58). In some embodiments, the nanobody has one or more conservative substitutions in SEQ ID NOs: 56, 57, and/or 58. For example, in some embodiments the disclosed TcdA-specific nanobody has the amino acid sequence: QVQLQESGGGTVQPGGSLRLSCAASGRDFSSGAMGWFRQTPGNEREFVGVGWSGGLIDYS DSVKGRFTITRDKLKNEVYLRMDRLKPEDTAVYFCAVNSANSCAGYDCHDKPQTYNYWGQGT QVTVSS (SEQ ID NO:59, A2B10), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:59.

In some embodiments, the nanobody comprises a variable domain having CDR1, CDR2 and CDR3 sequences. For example, in some embodiments, the CDR1 sequence comprises the amino acid sequence ERTFSAYT (SEQ ID NO:60); CDR2 sequence of the variable domain comprises the amino acid sequence IKWSGSGGIT (SEQ ID NO:61); and the CDR3 sequence of the variable domain comprises the amino acid sequence TAGPTVYNPHY (SEQ ID NO:62). In some embodiments, the nanobody has one or more conservative substitutions in SEQ ID NOs: 60, 61, and/or 62. For example, in some embodiments the disclosed TcdA-specific nanobody has the amino acid sequence: QVQLQESGGGLVQAGGSLRLSCTPSERTFSAYTMAWYRQPPGKEREFAAGIKWSGSGGITLY ADSVKGRFTISGDNAKSTVYLEMNSLKPEDTAVYYCTAGPTVYNPHYWGQGTQVTVSS (SEQ ID NO:63, A2B5), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:63.

In some embodiments, the nanobody comprises a variable domain having CDR1, CDR2 and CDR3 sequences. For example, in some embodiments, the CDR1 sequence comprises the amino acid sequence GRTFSRYA (SEQ ID NO:64); CDR2 sequence of the variable domain comprises the amino acid sequence ISYSGGTT (SEQ ID NO:65); and the CDR3 sequence of the variable domain comprises the amino acid sequence AAGLFRGDLTRFTLDEYDY (SEQ ID NO:66). In some embodiments, the nanobody has one or more conservative substitutions in SEQ ID NOs: 64, 65, and/or 66. For example, in some embodiments the disclosed TcdA-specific nanobody has the amino acid sequence: QVQLQESGGGLVQTGGSLRLSCTASGRTFSRYAMGWFRQASGKEREFVAAISYSGGTTYYSD SVKGRFTISRDNAKSTVSLQMNSLKPEDTAVYYCAAGLFRGDLTRFTLDEYDYRGQGTQVTVS S (SEQ ID NO:67, A2C2), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:67.

In some embodiments, the nanobody comprises a variable domain having CDR1, CDR2 and CDR3 sequences. For example, in some embodiments, the CDR1 sequence comprises the amino acid sequence GRSFSSGA (SEQ ID NO:68); CDR2 sequence of the variable domain comprises the amino acid sequence VGWSGGLI (SEQ ID NO:69); and the CDR3 sequence of the variable domain comprises the amino acid sequence AINSANSCAGYDCHDKPQVYDY (SEQ ID NO:70). In some embodiments, the nanobody has one or more conservative substitutions in SEQ ID NOs: 68, 69, and/or 70. For example, in some embodiments the disclosed TcdA-specific nanobody has the amino acid sequence: QVQLQESGGGLVQPGGSLRLSCAASGRSFSSGAMGWFRQAPGKEREFVGVGWSGGLIDYAA SVRGRFTITRDKAKNTVYLRMDSLRPEDTAVYYCAINSANSCAGYDCHDKPQVYDYWGQGTQ VTVSS (SEQ ID NO:71, A2F10), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:71.

In some embodiments, the nanobody comprises a variable domain having CDR1, CDR2 and CDR3 sequences. For example, in some embodiments, the CDR1 sequence comprises the amino acid sequence GRTFSRYA (SEQ ID NO:72); CDR2 sequence of the variable domain comprises the amino acid sequence ISWSGDTT (SEQ ID NO:73); and the CDR3 sequence of the variable domain comprises the amino acid sequence AAGLFRGDLTKFELDEYDY (SEQ ID NO:74). In some embodiments, the nanobody has one or more conservative substitutions in SEQ ID NOs: 72, 73, and/or 74. For example, in some embodiments the disclosed TcdA-specific nanobody has the amino acid sequence: QVQLQESGGGLVQAGGSLRLSCAASGRTFSRYAMGWFRQAPGKEREFVAAISWSGDTTYDA DSVKGRFTVSRDNAKNTVYLQMNSLKPEDTAVYYCAAGLFRGDLTKFELDEYDYRGQGTQVT VSS (SEQ ID NO:75, A2F12), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:75.

In some embodiments, the nanobody comprises a variable domain having CDR1, CDR2 and CDR3 sequences. For example, in some embodiments, the CDR1 sequence comprises the amino acid sequence GHSFSTSA (SEQ ID NO:76); CDR2 sequence of the variable domain comprises the amino acid sequence ISWAGGKI (SEQ ID NO:77); and the CDR3 sequence of the variable domain comprises the amino acid sequence AANSQNMCSGWDCEKQPRVYDF (SEQ ID NO:78). In some embodiments, the nanobody has one or more conservative substitutions in SEQ ID NOs: 76, 77, and/or 78. For example, in some embodiments the disclosed TcdA-specific nanobody has the amino acid sequence: QVQLQESGGGLVQPGGSLTLSCIASGHSFSTSAMAWFRQAPGKEREVVGISWAGGKIDYADFV RGRFTISRDNAKNTVSLQMNGLKPEETAVYYCAANSQNMCSGWDCEKQPRVYDFWGQGTQV TVSS (SEQ ID NO:79, A2G1), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:79.

In some embodiments, the nanobody comprises a variable domain having CDR1, CDR2 and CDR3 sequences. For example, in some embodiments, the CDR1 sequence comprises the amino acid sequence GRTLSSYA (SEQ ID NO:80); CDR2 sequence of the variable domain comprises the amino acid sequence ISRGGGMT (SEQ ID NO:81); and the CDR3 sequence of the variable domain comprises the amino acid sequence AASYALIDMSSAYDY (SEQ ID NO:82). In some embodiments, the nanobody has one or more conservative substitutions in SEQ ID NOs: 80, 81, and/or 82. For example, in some embodiments the disclosed TcdA-specific nanobody has the amino acid sequence: QVQLQESGGGLVQAGGSLRLSCAVSGRTLSSYAMGWFRQALGKEREFVAGISRGGGMTRYT DSVKGRFTISRDDAKNTVYLQMNSLKPDDTAVYSCAASYALIDMSSAYDYWGQGTQVTVSS (SEQ ID NO:83, A2G5), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:83.

In some embodiments, the nanobody comprises a variable domain having CDR1, CDR2 and CDR3 sequences. For example, in some embodiments, the CDR1 sequence comprises the amino acid sequence GRTFSRIA (SEQ ID NO:84); CDR2 sequence of the variable domain comprises the amino acid sequence ISGNGGT (SEQ ID NO:85); and the CDR3 sequence of the variable domain comprises the amino acid sequence AADPNYRATYFPYGMDY (SEQ ID NO:86). In some embodiments, the nanobody has one or more conservative substitutions in SEQ ID NOs: 84, 85, and/or 86. For example, in some embodiments the disclosed TcdA-specific nanobody has the amino acid sequence: QVQLQESGGGLVQAGGSLRLSCAASGRTFSRIAMGWFRQVPGKEREFVAGISGNGGTFYTDS VKGRFTISRDNTKNTVYLQMNSLRPEDTAVYYCAADPNYRATYFPYGMDYYGRGTQVTVSS (SEQ ID NO:87, A2G6), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:87.

In some embodiments, the nanobody comprises a variable domain having CDR1, CDR2 and CDR3 sequences. For example, in some embodiments, the CDR1 sequence comprises the amino acid sequence GSIFSINA (SEQ ID NO:88); CDR2 sequence of the variable domain comprises the amino acid sequence ITSGGTT (SEQ ID NO:89); and the CDR3 sequence of the variable domain comprises the amino acid sequence NLPWTSDLGWAVKDY (SEQ ID NO:90). In some embodiments, the nanobody has one or more conservative substitutions in SEQ ID NOs: 88, 89, and/or 90 For example, in some embodiments the disclosed TcdA-specific nanobody has the amino acid sequence: QVQLQESGGGLVQPGGSLRLSCAASGSIFSINAMGWYRQAPGKQRELVAKITSGGTTNYADSV KGRFTISRDGAKNTVYLQMNSLKPEDTAVYYCNLPWTSDLGWAVKDYWGQGTQVTVSS (SEQ ID NO:91, A2H4), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:91.

In some embodiments, the nanobody comprises a variable domain having CDR1, CDR2 and CDR3 sequences. For example, in some embodiments, the CDR1 sequence comprises the amino acid sequence GRASSTYV (SEQ ID NO:92); CDR2 sequence of the variable domain comprises the amino acid sequence DTWGGAGT (SEQ ID NO:93); and the CDR3 sequence of the variable domain comprises the amino acid sequence AAGQGRSVTLFQPSTYDY (SEQ ID NO:94). In some embodiments, the nanobody has one or more conservative substitutions in SEQ ID NOs: 92, 93, and/or 94 For example, in some embodiments the disclosed TcdA-specific nanobody has the amino acid sequence: QVQLQESGGGLVQAGGSLRLSCAASGRASSTYVMAWFRQAPGKEREFVAADTWGGAGTYYA PSVKGRFTISRDNAKNMLYLQMNSLKPEDTAAYYCAAGQGRSVTLFQPSTYDYWGQGTQVTV SS (SEQ ID NO:95, A2H9), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:95.

In some embodiments, the nanobody comprises a variable domain having CDR1, CDR2 and CDR3 sequences. For example, in some embodiments, the CDR1 sequence comprises the amino acid sequence TSINIYP (SEQ ID NO:96); CDR2 sequence of the variable domain comprises the amino acid sequence VNRDGNT (SEQ ID NO:97); and the CDR3 sequence of the variable domain comprises the amino acid sequence NNFGSSS (SEQ ID NO:98). In some embodiments, the nanobody has one or more conservative substitutions in SEQ ID NOs: 96, 97, and/or 98. For example, in some embodiments the disclosed TcdB-specific nanobody has the amino acid sequence: QVQLQESGGGLVQPGGSLRLTCTSSTSINIYPYMGWYRQAPGKQRERVATVNRDGNTNYLDS VKGRFTITRDDAKKTICLQMNNLEPEDTAVYYCNNFGSSSWGQGTQVTVSS (SEQ ID NO:99, B0A9), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:99.

In some embodiments, the nanobody comprises a variable domain having CDR1, CDR2 and CDR3 sequences. For example, in some embodiments, the CDR1 sequence comprises the amino acid sequence RSIDIYVA (SEQ ID NO:100); CDR2 sequence of the variable domain comprises the amino acid sequence IHRGDTT (SEQ ID NO:101); and the CDR3 sequence of the variable domain comprises the amino acid sequence NDFGGTR (SEQ ID NO:102). In some embodiments, the nanobody has one or more conservative substitutions in SEQ ID NOs: 100, 101, and/or 102. For example, in some embodiments the disclosed TcdB-specific nanobody has the amino acid sequence: QVQLQESGGALVQPGGSLRLTCTSSRSIDIYVAMAWYRQAPGKQRERVATIHRGDTTNYSDSV KGRFTISRDNAKNTITLQMNNLKPEDTAVYYCNDFGGTRWGQGTQVTVSS (SEQ ID NO:103, B0A12), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:103.

In some embodiments, the nanobody comprises a variable domain having CDR1, CDR2 and CDR3 sequences. For example, in some embodiments, the CDR1 sequence comprises the amino acid sequence GSIYGMMMMA (SEQ ID NO:104); CDR2 sequence of the variable domain comprises the amino acid sequence FTRDGSTNY (SEQ ID NO:105); and the CDR3 sequence of the variable domain comprises the amino acid sequence NIQRY (SEQ ID NO:106). In some embodiments, the nanobody has one or more conservative substitutions in SEQ ID NOs: 104, 105, and/or 106. For example, in some embodiments the disclosed TcdB-specific nanobody has the amino acid sequence:

QVQLQESGGGSVQPGGSLRLSCAASGSIYGMMMMAWYRQRTGEQRELVANFTRDGSTNYV DSVKGRFTISRDNDKKMVYLQMKDLKPEDTAVYYCNIQRYWGKGTAVTVSS (SEQ ID NO:107, B0B7), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:107.

In some embodiments, the nanobody comprises a variable domain having CDR1, CDR2 and CDR3 sequences. For example, in some embodiments, the CDR1 sequence comprises the amino acid sequence GFYFPNYA (SEQ ID NO:108); CDR2 sequence of the variable domain comprises the amino acid sequence ITSAGGST (SEQ ID NO:109); and the CDR3 sequence of the variable domain comprises the amino acid sequence NADPSYGTRY (SEQ ID NO:110). In some embodiments, the nanobody has one or more conservative substitutions in SEQ ID NOs: 108, 109, and/or 110. For example, in some embodiments the disclosed TcdB-specific nanobody has the amino acid sequence: QVQLQESGGGLVQPGGSLRLSCATSGFYFPNYAMSWHRQAPGQERELVARITSAGGSTDYAD SVKGRFTISRDNSKNTVYLQMNGLISEDTAVYYCNADPSYGTRYWGQGTQVTVSS (SEQ ID NO:111, B0B11), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:111.

In some embodiments, the nanobody comprises a variable domain having CDR1, CDR2 and CDR3 sequences. For example, in some embodiments, the CDR1 sequence comprises the amino acid sequence GFTRKHYT (SEQ ID NO:112); CDR2 sequence of the variable domain comprises the amino acid sequence ITTPDNST (SEQ ID NO:113); and the CDR3 sequence of the variable domain comprises the amino acid sequence GASALGGSSCAQSSSVLHRLFQ (SEQ ID NO:114). In some embodiments, the nanobody has one or more conservative substitutions in SEQ ID NOs: 112, 113, and/or 114. For example, in some embodiments the disclosed TcdB-specific nanobody has the amino acid sequence: QVQLQESGGGLVQPGGSLRLSCTASGFTRKHYTIGWFRQAPGKEREGVSCITTPDNSTYYKD SVKGRFTISRDNAKNTVYLQMNNVKPEDTAVYYCGASALGGSSCAQSSSVLHRLFQWGQGTQ VTVSS (SEQ ID NO:115, BOC10), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:115.

In some embodiments, the nanobody comprises a variable domain having CDR1, CDR2 and CDR3 sequences. For example, in some embodiments, the CDR1 sequence comprises the amino acid sequence GSLPSDYV (SEQ ID NO:116); CDR2 sequence of the variable domain comprises the amino acid sequence ITTADIT (SEQ ID NO:117); and the CDR3 sequence of the variable domain comprises the amino acid sequence KITILPSVSVY (SEQ ID NO:118). In some embodiments, the nanobody has one or more conservative substitutions in SEQ ID NOs: 116, 117, and/or 118. For example, in some embodiments the disclosed TcdB-specific nanobody has the amino acid sequence: QVQLQESGGGLVQPGGSLRLSCAASGSLPSDYVTSWHRQAPGKQRELVASITTADITNYAASV KGRFTISRDRAKNMGYLQMNSLLPEDTAVYYCKITILPSVSVYWGQGTQVTVSS (SEQ ID NO:119, B0D3), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:119.

In some embodiments, the nanobody comprises a variable domain having CDR1, CDR2 and CDR3 sequences. For example, in some embodiments, the CDR1 sequence comprises the amino acid sequence GFSLDYLA (SEQ ID NO:120); CDR2 sequence of the variable domain comprises the amino acid sequence IRSSDGTI (SEQ ID NO:121); and the CDR3 sequence of the variable domain comprises the amino acid sequence GIQAGGSTGDIRLACGGMDH (SEQ ID NO:122). In some embodiments, the nanobody has one or more conservative substitutions in SEQ ID NOs: 120, 121, and/or 122. For example, in some embodiments the disclosed TcdB-specific nanobody has the amino acid sequence: QVQLQESGGGLVQPGGSLRLSCAASGFSLDYLAIGWFRQAPGKGREGVSCIRSSDGTIFYSDS VKGRFTMSRDNAKNTVYLQMNSLKPEDTAVYHCGIQAGGSTGDIRLACGGMDHWGNGTQVT VSS (SEQ ID NO:123, B0D10), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:123.

In some embodiments, the nanobody comprises a variable domain having CDR1, CDR2 and CDR3 sequences. For example, in some embodiments, the CDR1 sequence comprises the amino acid sequence GFTFSHAV (SEQ ID NO:124); CDR2 sequence of the variable domain comprises the amino acid sequence WRSAGGIT (SEQ ID NO:125); and the CDR3 sequence of the variable domain comprises the amino acid sequence KAFVVGSAY (SEQ ID NO:126). In some embodiments, the nanobody has one or more conservative substitutions in SEQ ID NOs: 124, 125, and/or 126. For example, in some embodiments the disclosed TcdB-specific nanobody has the amino acid sequence: QVQLQESGGGLAQPGGSLRLSCAASGFTFSHAVMSWFRQAPGKERELVAGWRSAGGITNYA DSVKGRFTISGDNAKDVVYLQMNSLKPEDTAVYYCKAFVVGSAYWGQGTQVTVSS (SEQ ID NO:127, B0D11), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:127.

In some embodiments, the nanobody comprises a variable domain having CDR1, CDR2 and CDR3 sequences. For example, in some embodiments, the CDR1 sequence comprises the amino acid sequence GTVFKIYV (SEQ ID NO:128); CDR2 sequence of the variable domain comprises the amino acid sequence ISNGGTP (SEQ ID NO:129); and the CDR3 sequence of the variable domain comprises the amino acid sequence NRRQLEGRQSEDY (SEQ ID NO:130). In some embodiments, the nanobody has one or more conservative substitutions in SEQ ID NOs: 128, 129, and/or 130. For example, in some embodiments the disclosed TcdB-specific nanobody has the amino acid sequence: QVQLQESGGGLVLPGGSLRLSCAASGTVFKIYVMGWYRQAPGKQRELVATISNGGTPNYADSV KGRFTISGDRAKNTVFLQMSSLNVEDTAVYYCNRRQLEGRQSEDYWGQGTQVTVSS (SEQ ID NO:131, B0E2), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:131.

In some embodiments, the nanobody comprises a variable domain having CDR1, CDR2 and CDR3 sequences. For example, in some embodiments, the CDR1 sequence comprises the amino acid sequence RSINIYVA (SEQ ID NO:132); CDR2 sequence of the variable domain comprises the amino acid sequence AHKDGGT (SEQ ID NO:133); and the CDR3 sequence of the variable domain comprises the amino acid sequence NAFGSSA (SEQ ID NO:134). In some embodiments, the nanobody has one or more conservative substitutions in SEQ ID NOs: 132, 133, and/or 134. For example, in some embodiments the disclosed TcdB-specific nanobody has the amino acid sequence: QVQLQESGGGVVQPGGSLRLTCTSSRSINIYVAMGWYRQAPGKQRERVATAHKDGGTRYSDS VKGRFTISRDDDKNTVYLQMNNLEPGDTAVYYCNAFGSSAWGQGTQVTVSS (SEQ ID NO:135, B1A11), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:135.

In some embodiments, the nanobody comprises a variable domain having CDR1, CDR2 and CDR3 sequences. For example, in some embodiments, the CDR1 sequence comprises the amino acid sequence GLSLSTDV (SEQ ID NO:136); CDR2 sequence of the variable domain comprises the amino acid sequence IRSAGWIT (SEQ ID NO:137); and the CDR3 sequence of the variable domain comprises the amino acid sequence KVLRLPDGLAF (SEQ ID NO:138). In some embodiments, the nanobody has one or more conservative substitutions in SEQ ID NOs: 136, 137, and/or 138. For example, in some embodiments the disclosed TcdB-specific nanobody has the amino acid sequence: QVQLQESGGGLVQPGGSLRLSCLASGLSLSTDVMSWFRQAPGKERELVAGIRSAGWITNYAD SVKGRFTISVDSAKNTVYLQMNSLKPEDTAVYYCKVLRLPDGLAFWGQGTQVTVSS (SEQ ID NO:139, B1C10), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:139.

In some embodiments, the nanobody comprises a variable domain having CDR1, CDR2 and CDR3 sequences. For example, in some embodiments, the CDR1 sequence comprises the amino acid sequence GFTFSGYG (SEQ ID NO:140); CDR2 sequence of the variable domain comprises the amino acid sequence STADSTP (SEQ ID NO:141); and the CDR3 sequence of the variable domain comprises the amino acid sequence RTRTAWEEY (SEQ ID NO:142). In some embodiments, the nanobody has one or more conservative substitutions in SEQ ID NOs: 140, 141, and/or 142 For example, in some embodiments the disclosed TcdB-specific nanobody has the amino acid sequence: QVQLQESGGGLVQPGGFLRLACAASGFTFSGYGVSWYRQAPGKEREFIAASTADSTPNYAGS VKGRFTISRDSAKNMVYLQMNNLKSEDTGVYYCRTRTAWEEYWGQGTQVTVSS (SEQ ID NO:143, B1C11), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:143.

In some embodiments, the nanobody comprises a variable domain having CDR1, CDR2 and CDR3 sequences. For example, in some embodiments, the CDR1 sequence comprises the amino acid sequence GSLRSGYV (SEQ ID NO:144); CDR2 sequence of the variable domain comprises the amino acid sequence ITTGDIT (SEQ ID NO:145); and the CDR3 sequence of the variable domain comprises the amino acid sequence KITELPTVSVY (SEQ ID NO:146). In some embodiments, the nanobody has one or more conservative substitutions in SEQ ID NOs: 144, 145, and/or 146. For example, in some embodiments the disclosed TcdB-specific nanobody has the amino acid sequence: QVQLQESGGGLVQPGGSLRLSCAASGSLRSGYVMSWHRQAPGKQRELVASITTGDITQYGAS VKGRFTISRDETKNMGWLQMNSLLPEDTAVYYCKITELPTVSVYWGQGTQVTVSS (SEQ ID NO:147, B1E7), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:147.

In some embodiments, the nanobody comprises a variable domain having CDR1, CDR2 and CDR3 sequences. For example, in some embodiments, the CDR1 sequence comprises the amino acid sequence GLTSGTYV (SEQ ID NO:148); CDR2 sequence of the variable domain comprises the amino acid sequence IRDAGGIR (SEQ ID NO:149); and the CDR3 sequence of the variable domain comprises the amino acid sequence KFLRLPESLAY (SEQ ID NO:150). In some embodiments, the nanobody has one or more conservative substitutions in SEQ ID NOs: 148, 149, and/or 150. For example, in some embodiments the disclosed TcdB-specific nanobody has the amino acid sequence: QVQLQESGGGLVQPGGSLRLSCAASGLTSGTYVMSWFRQAPGKEREFVAAIRDAGGIRNYAD SVKGRFTISRDAAKNMIFLQMNSLKPEDTAVYYCKFLRLPESLAYWGQGTQVTVSS (SEQ ID NO:151, B2C5), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:151.

In some embodiments, the nanobody comprises a variable domain having CDR1, CDR2 and CDR3 sequences. For example, in some embodiments, the CDR1 sequence comprises the amino acid sequence GSIFGVNT (SEQ ID NO:152); CDR2 sequence of the variable domain comprises the amino acid sequence ISPGGYT (SEQ ID NO:153); and the CDR3 sequence of the variable domain comprises the amino acid sequence NYRSGTSRPNTN (SEQ ID NO:154). In some embodiments, the nanobody has one or more conservative substitutions in SEQ ID NOs: 152, 153, and/or 154. For example, in some embodiments the disclosed TcdB-specific nanobody has the amino acid sequence: QVQLQESGGGLVHVGESLTLSCVAPGSIFGVNTMAWYRQAPGKQRELVAAISPGGYTNYAEFV KGRFGISRDKAKNTVYLQMNDLKPEDTAVYYCNYRSGTSRPNTNWGQGTQVTVSS (SEQ ID NO:155, B2C11), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:155.

In some embodiments, the nanobody comprises a variable domain having CDR1, CDR2 and CDR3 sequences. For example, in some embodiments, the CDR1 sequence comprises the amino acid sequence GFTFSNYV (SEQ ID NO:156); CDR2 sequence of the variable domain comprises the amino acid sequence VSESGQSR (SEQ ID NO:157); and the CDR3 sequence of the variable domain comprises the amino acid sequence NTGSRTYGARY (SEQ ID NO:158). In some embodiments, the nanobody has one or more conservative substitutions in SEQ ID NOs: 156, 157, and/or 158. For example, in some embodiments the disclosed TcdB-specific nanobody has the amino acid sequence: QVQLQESGGGLVQAGGSLTLSCTASGFTFSNYVMSWYRQAPGKEREWVASVSESGQSRTYA DSVKGRFTISRDNTNFSVYLQMNNLNSEDTAVYYCNTGSRTYGARYWGQGTQVTVSS (SEQ ID NO:159, B2F11), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:159.

In the present invention, “a conservative substitution” refers to 1, 2, 3, 4, 5, or 6 amino acids substituted by amino acids having analogical or similar properties, compared to the amino acid sequence of the nanobody of the present invention. These conservative substitutions can, for example, be produced according to the amino acid substitutions in Table 1.

TABLE 1
Conservative Substitutions

| Original residue | substitution | substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

Also disclosed herein is a fusion protein comprising the disclosed nanobodies or fragments thereof. In addition to almost full-length polypeptides, the present invention also includes fragments of the nanobodies of the invention. Typically, the fragment has at least about 50 contiguous amino acids of the disclosed nanobody, preferably at least about 50 contiguous amino acids, more preferably at least about 80 contiguous amino acids, and most preferably at least about 100 contiguous amino acids.

In some embodiments, the nanobody disclosed herein is conjugated to a cell penetrating peptide (CPP). Non-limiting examples of CPPs include the HIV-1 TAT translocation domain (Green; M. and Loewenstein, P. M. (1988) Cell 55, 1179-1188) and the homeodomain of the Antennapedia protein from Drosophila (Joliot; A. et al. (1991) Proc. Natl. Acad. Sci. USA 88, 1864-1868); a sequence of 16 amino acids called penetratin or pAntp of the Antennapedia protein (Derossi, D. et al. (1994) J. Biol. Chem. 269, 10444-10450); a basic sequence of the HIV-1 Tat protein (Vives, E. et al. (1997) J. Biol. Chem. 272, 16010-16017); and a synthetic peptide developed is the amphipathic model peptide MAP (Oehlke, J. et al. (1998) Biochim. Biophys. Acta 1414, 127-139). Additional non-limiting examples of CPPs are described in U.S. Pat. Nos. 9,303,076; and 9,302,014. Examples of linear CPPs are provided in Table 2.

TABLE 2
Linear CPPs

| | | |
|---|---|---|
| cpp1 | IRRGISRK | SEQ ID NO: 161 |
| cpp2 | KRKRAV | SEQ ID NO: 162 |
| cpp3 | PKPKRQTKR | SEQ ID NO: 163 |
| cpp4 | RRRRHCNR | SEQ ID NO: 164 |
| cpp5 | IPDPTGQS | SEQ ID NO: 165 |
| cpp6 | IKREREND | SEQ ID NO: 166 |
| cpp7 | KKLQEQEKQQKVEFRKR | SEQ ID NO: 167 |
| cpp8 | GPNKKKRKL | SEQ ID NO: 168 |
| cpp9 | RRRRASAPISQWSSSRRSR | SEQ ID NO: 169 |
| cpp10 | KLALKLALKALKAALKLA | SEQ ID NO: 170 |
| cpp11 | FLPLIGRVLSGIL | SEQ ID NO: 171 |
| cpp12 | CVQWSLLRGYQPCCVQWSLLRGYQPC | SEQ ID NO: 172 |
| cpp13 | RKKRKGSGSRKKRKGSGSRKKRKGSGSRKKRK | SEQ ID NO: 173 |
| cpp14 | CVQWSLLRGYQPCGSGSCVQWSLLRGYQPC | SEQ ID NO: 174 |
| cpp15 | KKRRRGSGSKKRRRGSGSKKRRRGSGSKKRRR | SEQ ID NO: 175 |
| cpp16 | RKKRRGSGSRKKRRGSGSRKKRRGSGSRKKRR | SEQ ID NO: 176 |
| cpp17 | RQIKIWFQNRRMKWKKC | SEQ ID NO: 177 |
| [GRKRKRS]4 | GRKRKRSGRKRKRSGRKRKRSGRKRKRS | SEQ ID NO: 178 |
| [PRKKRGR]4 | PRKKRGRPRKKRGRPRKKRGRPRKKRGR | SEQ ID NO: 179 |
| CPP Motif 1 | CVQWSLLRGYQPC | SEQ ID NO: 180 |
| CPP Motif 2 | XKXRX-GSGS, where X is R or K | SEQ ID NO: 181 |
| 6xHis | HHHHHH | SEQ ID NO: 182 |
| HIV-TAT | RKKRRQRRR | SEQ ID NO: 183 |
| HIV-REV | RQARRNRRRRWR | SEQ ID NO: 184 |
| NLS | PKKKRKV | SEQ ID NO: 185 |
| dodecahistidine | HHHHHHHHHHHH | SEQ ID NO: 186 |

In some embodiments, the CPP is a cyclic CPP. Examples of cyclic CPPs are described in US/20210070806, which is incorporated by references in its entirety for the teaching of these CPPs. Examples of cyclic CPPs are provided in Table 3.

TABLE 3
cyclic CPPs

| | | |
|---|---|---|
| CPP1 | cyclo(FΦRRRRQ) | SEQ ID NO: 187 |
| CPP9 | cyclo(fΦRrRrQ) | SEQ ID NO: 188 |
| CPP12 | cyclo(FfΦRrRrQ) | SEQ ID NO: 189 |
| CPP1-1 | cyclo(FtBuRRRRQ) | SEQ ID NO: 190 |
| CPP1-2 | cyclo(DapHexanRRRRQ) | SEQ ID NO: 191 |
| CPP1-3 | cyclo(DapOctanRRRRQ) | SEQ ID NO: 192 |
| CPP1-4 | cyclo(DapDecaRRRRQ) | SEQ ID NO: 193 |
| CPP1-5 | cyclo(Dap1-PyrenRRRRQ) | SEQ ID NO: 194 |
| CPP1-6 | cyclo(Dap3,3-diphenyRRRRQ) | SEQ ID NO: 195 |
| CPP1-7 | cyclo(DapFmocRRRRQ) | SEQ ID NO: 196 |
| CPP1-8 | cyclo(Dap1-PyrenebRRRRQ) | SEQ ID NO: 197 |
| CPP1-10 | cyclo(DapDecaRrRrQ) | SEQ ID NO: 198 |
| CPP1-11 | cyclo(DapDecarRrRQ) | SEQ ID NO: 199 |
| CPP1-12 | cyclo(DapDecaARRRQ) | SEQ ID NO: 200 |
| CPP1-13 | cyclo(DapDecaRRRAQ) | SEQ ID NO: 201 |
| CPP1-14 | cyclo(DapDecaRRRRRQ) | SEQ ID NO: 202 |
| CPP1-15 | cyclo(LysDecaRRRRQ) | SEQ ID NO: 203 |
| CPP1-16 | cyclo(DapDecaRRRQ) | SEQ ID NO: 204 |
| CPP1-17 | cyclo(OrnDecaRRRRQ) | SEQ ID NO: 205 |
| CPP1-18 | cyclo(LysDecaRrRrQ) | SEQ ID NO: 206 |
| CPP1-19 | cyclo(LysDecarRrRQ) | SEQ ID NO: 207 |
| CPP1-20 | cyclo(AspDecyRRRRQ) | SEQ ID NO: 208 |
| CPP1-22 | cyclo(AspDecyRrRrQ) | SEQ ID NO: 209 |
| CPP1-23 | cyclo(GluDecyRrRrQ) | SEQ ID NO: 210 |
| CPP1-24 | cyclo(AspDecyrRrRQ) | SEQ ID NO: 211 |
| CPP1-25 | cyclo(GluDecyrRrRQ) | SEQ ID NO: 212 |

*single letter abbreviations: when shown in capital letters herein it indicates the L-amino acid form, when shown in lower case herein it indicates the D-amino acid form. Φ = 2-naphthylalanine, Dap = 2,3-diaminopropionic acid, tBu = tert-butanoyl, hexan = hexanoyl, octan = octanoyl, deca = decanoyl, 1-pyren = pyrenol, 3,3-diphenyl = 3,3-diphenoyl, Fmoc = fluorenylmethyloxycarbonoyl, 1-pyreneb = 1-pyrenylbutanoyl, decy = decynoyl

In some embodiments, the nanobody may be subjected to an alteration to render it less immunogenic when administered to a human. Such an alteration may comprise one or more of the techniques commonly known as chimerization, humanization, CDR-grafting, deimmunization and/or mutation of framework region amino acids to correspond to the closest human germline sequence (germlining). Bispecific antibodies which have been altered will therefore remain administrable for a longer period of time with reduced or no immune response-related side effects than corresponding bispecific antibodies which have not undergone any such alteration(s). One of ordinary skill in the art will understand how to determine whether, and to what degree a nanobody must be altered in order to prevent it from eliciting an unwanted host immune response.

Also disclosed herein is a polynucleotide molecule encoding the above nanobody or fragment or fusion protein thereof. Polynucleotides of the invention may be in the form of DNA or RNA. DNA forms include cDNA, genomic DNA, or synthetic DNA. DNA can be single-stranded or double-stranded. DNA can be a coding strand or a non-coding strand.

### Detectable Moiety

In some embodiments, the nanobody disclosed herein is conjugated to a detectable moiety. The detectable moiety can be any moiety that is capable of producing, either directly or indirectly, a detectable signal. Examples of detectable moieties for antibodies include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, beta-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance. Such antibodies and their fragments may be used for diagnostic applications, including but not limited to detection applications and imaging applications.

In some embodiments, a detectable moiety comprises a fluorophore. Representative fluorophores include, but are not limited to 7-dimethylaminocoumarin-3-carboxylic acid, dansyl chloride, nitrobenzodiazolamine (NBD), dabsyl chloride, cinnamic acid, fluorescein carboxylic acid, Nile Blue, tetramethylcarboxyrhodamine, tetraethyl sulfohodamine, 5-carboxy-X-rhodamine (5-ROX), and 6-carboxy-X-rhodamine (6-ROX). It is understood that these representative fluorophores are exemplary only, and additional fluorophores can also be employed. For example, there the ALEXA FLUOR® dye series includes at least 19 different dyes that are characterized by different emission spectra. These dyes include ALEXA FLUOR® 350, 405, 430, 488, 500, 514, 532, 546, 555, 568, 594, 610, 633, 635, 647, 660, 680, 700, and 750 (available from Invitrogen Corp., Carlsbad, Calif., United States of America), and the choice of which dye to employ can be made by the skilled artisan after consideration of the instant specification based on criteria including, but not limited to the chemical compositions of the specific ALEXA FLUORO, whether multiple detectable moieties are to be employed and the emission spectra of each, the detection technique to be employed, etc.

In some embodiments, a detectable moiety is a cyanine dye. Non-limiting examples of cyanine dyes that can be conjugated to the antibody fragments of the presently disclosed subject matter include the succinimide esters CyS, CyS.5, and Cy7, supplied by Amersham Biosciences (Piscataway, N.J., United States of America).

In some embodiments, a detectable moiety comprises a near infrared (NIR) dye. Non-limiting examples of near infrared dyes that can be conjugated to the antibody fragment of the presently disclosed subject matter include NIR641, NIR664, NIT7000, and NIT782.

### Pharmaceutical Composition

Also disclosed is a pharmaceutical composition comprising a disclosed nanobody in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, PA. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutically acceptable carriers include any and all suitable solvents, dispersion media, coatings, antibacterial and antifungal agents, isotonicity agents, antioxidants and absorption delaying agents, and the like that are physiologically compatible with a bispecific antibody of the present invention. Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, carboxymethyl cellulose colloidal solutions, tragacanth gum and injectable organic esters, such as ethyl oleate, and/or various buffers. Pharmaceutically acceptable carriers include sterile aqueous solutions or dispersions and sterile powders for the extemporaneous preparation of sterile injectable solutions or dispersion. Proper fluidity may be maintained, for example, by the use of coating materials, such as lecithin, by the maintenance of the required particle size in the case of dispersions, and by the use of surfactants.

Pharmaceutical nanobody may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole, butylated hydroxytoluene, lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical nanobody may also comprise isotonicity agents, such as sugars, polyalcohols, such as mannitol, sorbitol, glycerol or sodium chloride in the compositions.

The pharmaceutical nanobody may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The bispecific antibodies may be prepared with carriers that will protect the bispecific antibody against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art.

Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Also disclosed is the use of a disclosed nanobody for use as a medicament for the treatment of human monocytic ehrlichiosis (HME).

### Methods of Use

Also disclosed is a method for reducing the risk of C. difficile infection and/or improving resistance to C. difficile infection, and/or reducing the severity of C. difficile infection and/or decreasing the amount of C. difficile toxin, comprising administering, to a subject in need of such treatment, an effective amount of a composition described herein. In certain embodiments, the method of reducing the severity or risk of C. difficile infection comprises reducing the severity or risk of a C. difficile-associated disease, as described herein.

Subjects in need of such treatment or compositions include subjects who are either at risk for developing C. difficile infection and/or subjects who have existing C. difficile infection.

Subjects at risk for C. difficile infection include individuals who are or have been treated with an antibiotic; individuals who are very young (juvenile) or who are old (geriatric, e.g. humans aged 65 years or older); individuals suffering from an inflammatory bowel disease or condition (including human inflammatory bowel disease IBD and Crohn's Disease); individuals who are hospitalized or in a long-term care facility or who have been, in the past 2, 3, 4, 5, or 6 weeks, hospitalized or in a long-term care facility; individuals with cancer including those undergoing anti-cancer treatment and/or stem cell or bone marrow transplant recipients; individuals who have previously suffered C. difficile infection, and individuals undergoing immunosuppressive therapy or with an otherwise compromised immune system (e.g. subjects infected with an immunodeficiency causing retrovirus such as HIV, FIV, FLV, etc.).

Non-limiting examples of antibiotics associated with risk of C. difficile infection include ß-lactam antibiotics such as penicillin, ampicillin, and amoxicillin; clindamycin; cephalosporins such as but not limited to cefixime; quinolone antibiotics such as ciprofloxacin, levofloxacin and fluoroquinolone; macrolide antibiotics; trimethoprim; or sulfonamide antibiotics. In one specific non-limiting embodiment, the antibiotic is not enrofloxacin.

C. difficile infection, as that term is used herein, is distinct from the mere presence of the bacterium or C. difficile spores in the host gastrointestinal tract; infection is indicated by the presence of one or more symptom, such as intestinal tenderness, pain, and/or cramping; diarrhea for example watery diarrhea occurring at least 3, at least 5, or at least 8 times per day; blood or pus in stool or diarrhea; fever; loss of appetite; and/or nausea; and/or one or more clinical sign such as an elevated white blood cell count, decreased serum albumin, and/or the appearance of pseudomembrane in the intestinal and/or rectal mucosa. In a specific, non-limiting embodiment, C. difficile infection in a human may be manifested (in the case of a serious infection) by a fever of at least 38.3° C., a white blood cell count of greater than 15,000 cells/mm³, serum albumin less than 2.5 g/dl, and age greater than 60 years (Zar et al., Clin. Infect. Dis. 45:302-307, 2007).

In certain non-limiting embodiments, a “C. difficile-associated disease” refers to any disease involving unwanted growth, toxin production, or tissue invasion in the bowel by C. difficile. C. difficile-associated diseases are known in the art and include antibiotic-associated diarrhea (i.e., C. difficile colitis), pseudomembranous colitis, and C. difficile-associated toxic megacolon. C. difficile colitis generally refers to profuse, watery diarrheal illness associated with the presence of at least one C. difficile toxin. In certain embodiments, pseudomembranous colitis refers to a severe form of C. difficile colitis further characterized by bloody diarrhea, fever, and bowel wall invasion by C. difficile. The appearance of pseudomembranes on the surface of the colon or rectum, or in the intestinal and/or rectal mucosa, is diagnostic of the condition. In certain embodiments, the pseudomembranes are composed principally of inflammatory debris and white blood cells.

In certain non-limiting embodiments, the present invention provides for a method for reducing the risk of C. difficile infection and/or improving resistance to C. difficile infection, comprising administering, to a subject in need of such treatment, an effective amount of a composition or therapeutic bacteria described herein, for example, C. scindens bacteria.

An effective amount of a composition described herein, for example, C. scindens, is an amount which increases resistance to C. difficile infection, reduces the amount of C. difficile toxin, and/or inhibits proliferation and/or growth of C. difficile in a subject.

In certain non-limiting embodiments, the present invention provides for a method for reducing the severity of C. difficile infection, and/or decreasing the amount of C. difficile toxin, and/or reducing the risk of C. difficile infection, and/or improving resistance to C. difficile infection, comprising administering, to a subject in need of such treatment, an effective amount of a composition described herein.

Reducing the severity of C. difficile infection refers to an amelioration in the clinical symptoms or signs of infection, for example, but not by way of limitation, one or more of the following: a decrease in the frequency or volume of diarrhea; a decrease in fever; a decrease in abdominal cramping, pain, and/or tenderness; a reduction in white blood cells in the blood; an increase in serum albumin; weight maintenance or gain; and a decrease the appearance of pseudomembrane in the intestinal and/or rectal mucosa.

A subject treated according to the invention may be concurrently or sequentially treated with one or more agent that reduces the risk of and/or ameliorates C. difficile infection, for example, but not limited to, one or more antibiotic for example, but not limited to, vancomycin, metronidazole, and/or fidaxomicin; an immunotherapeutic agent such as an anti-toxin antibody; an herbal remedy such as Puerariae radix, Scutellariae radix, Rhizoma coptidis, garlic, or one or more extract thereof; and/or a probiotic remedy including for example, but not limited to, Lactobaccilus acidophilus, Lactobacillus casei, Bifidobacteriua, Streptococcus thermophiles, and/or Saccharomyces boulardii.In certain non-limiting embodiments, the treatment does not further comprise administration of cholestyramine.

The present disclosure also provides for methods of diagnosing or identifying a subject with a C. difficile infection, or at risk for C. difficile infection.

In certain embodiments, such methods comprise determining the level of one more bacterium present in an intestinal microbiota sample of a subject that can convert a primary bile acid to a secondary bile acid, for example, C. scindens, wherein the subject is diagnosed or identified as having a C. difficile infection, or at risk for C. difficile infection, when the level or amount of the one or more bacterium in the subject's microbiota is lower than a bacterium reference level. In one embodiment, a bacterium reference level is a level of bacterium, for example, C. scindens or any other bacteria that can convert a primary bile acid to a secondary bile acid, present in intestinal microbiota, a level below which is indicative of C. difficile infection, or risk of C. difficile infection, as determined by a medical doctor or person of skill in the art. In one non-limiting example, such a reference level can be the level of said bacterium in the microbiota of a subject who does not have a C. difficile infection, or at risk for C. difficile infection.

In other embodiments, such methods comprise determining the activity or level of 7α-hydroxysteroid dehydrogenase enzyme present in the intestinal microbiota of a subject, wherein the subject is diagnosed or identified as having a C. difficile infection, or at risk for C. difficile infection, when the activity or level of 7α-hydroxysteroid dehydrogenase enzyme in the subject's microbiota is lower than a 7α-hydroxysteroid dehydrogenase enzyme reference level. In one embodiment, a 7α-hydroxysteroid dehydrogenase enzyme reference level is an activity or level of 7α-hydroxysteroid dehydrogenase enzyme present in intestinal microbiota, a level or activity below which is indicative of C. difficile infection, or risk of C. difficile infection, as determined by a medical doctor or person of skill in the art. In one non-limiting example, such a reference level can be the activity or level of 7α-hydroxysteroid dehydrogenase in the microbiota of a subject who does not have a C. difficile infection, or at risk for C. difficile infection.

In other embodiments, such methods comprise quantifying the level of bile acid-inducible (bai) 7α/ß-dehydroxylation operon nucleic acid present in a fecal sample of a subject, wherein the subject is diagnosed or identified as having a C. difficile infection, or at risk for C. difficile infection, when the level of bile acid-inducible (bai) 7α/ß-dehydroxylation operon nucleic acid present in the fecal sample is lower than a bile acid-inducible (bai) 7α/ß-dehydroxylation operon nucleic acid reference level. In one embodiment, a bile acid-inducible (bai) 7α/ß-dehydroxylation operon nucleic acid reference level is the level of bile acid-inducible (bai) 7α/ß-dehydroxylation operon nucleic acid present in a fecal sample, a level below which is indicative of C. difficile infection, or risk of C. difficile infection, as determined by a medical doctor or person of skill in the art. In one non-limiting example, such a reference level can be the level of bile acid-inducible (bai) 7α/ß-dehydroxylation operon nucleic acid present in a fecal sample of a subject who does not have a C. difficile infection, or at risk for C. difficile infection. In certain embodiments, the level of nucleic acid is quantified using metagenomic sequencing, quantitative PCR, or any other method known in the art for quantifying nucleic acid in a sample.

In certain embodiments, when the level or activity of the one more bacterium present in an intestinal microbiota sample of a subject that can convert a primary bile acid to a secondary bile acid, the 7α-hydroxysteroid dehydrogenase enzyme in the subject's microbiota, and/or the level of bile acid-inducible (bai) 7α/ß-dehydroxylation operon nucleic acid present in the fecal sample is above their respective reference levels, the subject is not administered an antibiotic selected from the group consisting of a ß-lactam antibiotic, clindamycin, a cephalosporin, a quinolone antibiotic, levofloxacin, fluoroquinolone, a macrolide antibiotic, trimethoprim, and a sulfonamide antibiotic.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Nanobodies Against C. difficile Large Clostridial Toxins Reveal Unexpected

### Results

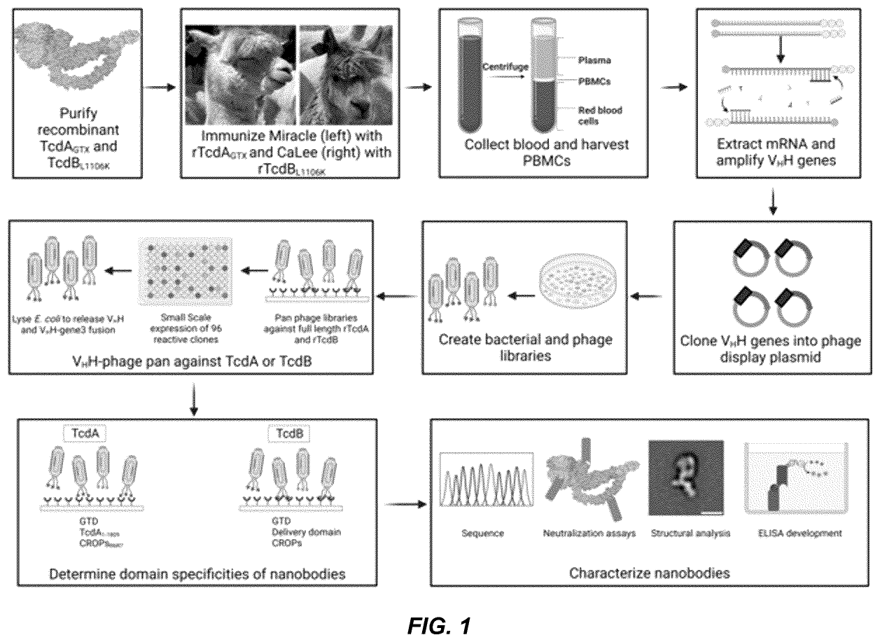

As outlined in FIG. 1, two alpacas were immunized with either TcdA_{GTX} (a glucosyltransferase-deficient mutant of TcdA) or TcdB L1106K, a non-toxic version of a TcdB1 sequence derived from the VPI10463 strain. ‘Miracle’ was immunized eight times with TcdAGTX, and ‘CaLee’ was similarly immunized with TcdB L1106K. Following immunization, peripheral blood was drawn, RNA was isolated, cDNA was made, and phage display libraries were produced. A single round of panning using TcdA_{GTX} and TcdB L1106K was done against each library, and reactive phage were recovered into E. coli and stored as glycerol stocks. From these libraries, deep-well 96 well plates were inoculated with single clones recovered from the pans (three plates from the TcdB L1106K pan and two from the TcdA_{GTX} pan), and protein expression was induced. Lysates from these clones were used in ELISA-like assays against either purified full-length toxins, or isolated domains. In total, each TcdA supernatant was screened against full-length TcdA, the TcdA glucosyltransferase domain (GTD), a GTD-APD-DD construct lacking the CROPS domain (TcdA₁₋₁₈₀₉) or repeats 6 and 7 of the TcdA CROPS (TcdA_{R6R7}). Each TcdB supernatant was screened against full-length TcdB1, the TcdB1 glucosyltransferase domain (GTD), the TcdB1 delivery domain (TcdB₈₄₂₋₁₈₃₄) or the TcdB CROPS domain (TcdB_{X-Y}). Clones encoding nanobodies that recognized full length TodA and TcdA₁₋₁₈₀₉ but failed to recognize TcdA GTD were tentatively assigned as APD-DD binders.

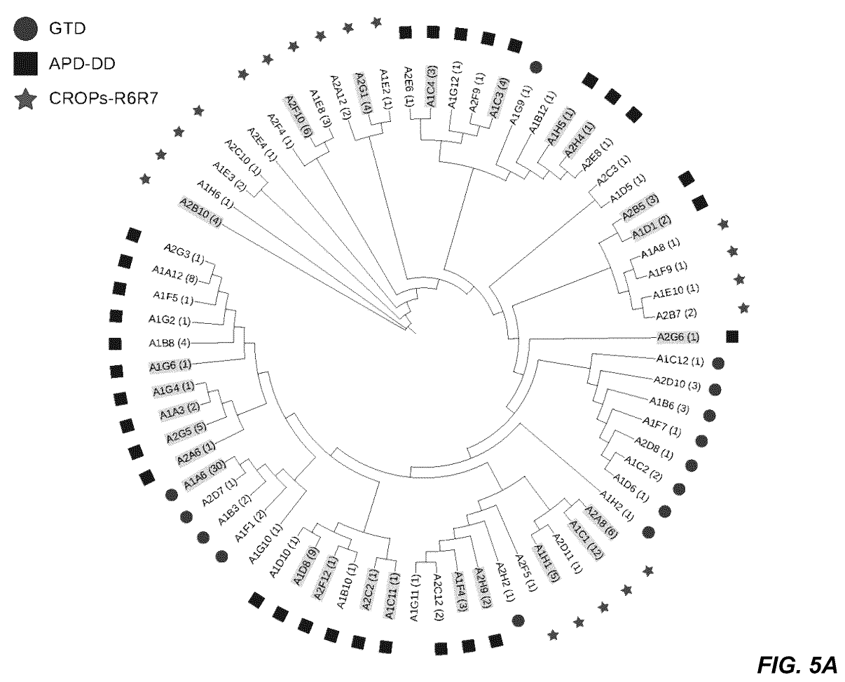

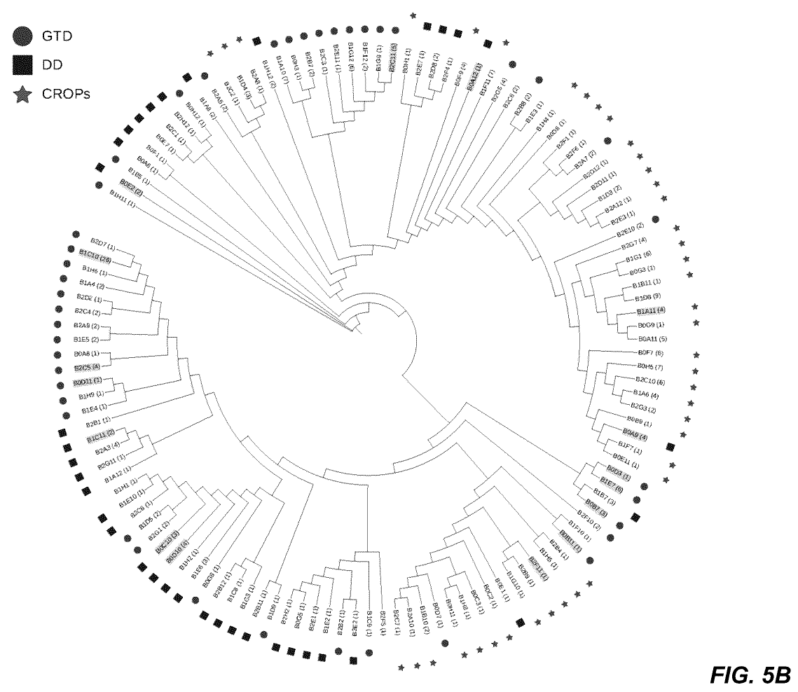

All clones (five 96 well plates) were sequenced, and cladograms were assembled based on the protein sequences (FIG. 5). The antigenic sequences were well-distributed across the length of the four-domain toxins (Table 1). TcdA_{GTX} immunization resulted in 69 unique clones: 14 against GTD, 30 against APD-DD, and 19 against CROPS R6R7. TcdB L1106K immunization resulted in 126 unique clones: 43 against GTD, 35 against DD, and 41 against CROPS. Sequence analysis identified substantial redundancy in the TcdA panel, such that only 69 of 176 recovered clones were unique, with some clones being particularly common. The TcdB panel was somewhat more diverse, with 126 of 267 clones being unique (Table 4). Each group had a few relatively dense clades (FIG. 5) with each group having a single very successful clone. For example, the A1A6 sequence (recovered from the TcdA immunized animal) was represented 30 times, while the B0A7 sequence (recovered from the TcdB immunized animal) was represented 26 times. Additional sequence analysis to map the likely germline origin of these clones revealed somewhat limited V gene usage, with a single V gene responsible for ˜75% of the clones identified from each animal. IGHV 3-3 was the inferred germline parent of 71% of TcdA clones, and IGHV 3S53 was the inferred germline parent of 79.4% of the TcdB clones. Inferred D gene usage was more variable, but inferred J gene usage also showed a small number of dominant gene segments. 98.9% of TcdA clones use TGHJ4, 44.2% of TcdB clones use IGHJ 6, and an additional 37.5% of TcdB clones use IGHJ4. The relatively constrained V gene usage highlights the importance of CDR3. A summary of these results is presented in Table 5.

TABLE 4

| | Total sequences | Non-redundant |
|---|---|---|
| TcdA | 176 | 69 |
| Domain specificity | | |
| GTD | 50 | 14 |
| APD-DD | 63 | 30 |
| CROPs | 54 | 19 |
| n.d | 9 | 6 |
| TcdB | 267 | 126 |
| Domain specificity | | |
| GTD | 106 | 43 |
| DD | 50 | 35 |
| CROPs | 98 | 41 |
| n.d | 13 | 7 |

n.d. = not determined

TABLE 5

| | V/D/J Gene | # Clones | Percent |
|---|---|---|---|
| TcdA (total clones = 176) | | | |
| | IGHV 3-3 | 125 | 71.0 |
| | IGHV 3S53 | 15 | 8.5 |
| | IGHV 3S61 | 3 | 1.7 |
| | IGHV 3S66 | 31 | 17.6 |
| | IGHV 3S65/3S66 | 2 | 1.1 |
| | IGHD 1 | 9 | 5.1 |
| | IGHD 2 | 29 | 16.5 |
| | IGHD 3 | 36 | 20.5 |
| | IGHD 4 | 32 | 18.2 |
| | IGHD 5 | 26 | 14.8 |
| | IGHD 6 | 1 | 0.6 |
| | IGHD 7 | 7 | 4.0 |
| | n.d. | 36 | 20.5 |
| | IGHJ 4 | 174 | 98.9 |
| | IGHJ 6 | 1 | 0.6 |
| | IGHJ 7 | 1 | 0.6 |
| | TcdB (total clones = 267) | | |
| | IGHV 3-3 | 5 | 1.9 |
| | IGHV 3S6 | 1 | 0.4 |
| | IGHV 3S9 | 1 | 0.4 |
| | IGHV 3S42 | 1 | 0.4 |
| | IGHV 3S53 | 212 | 79.4 |
| | IGHV 3S54 | 2 | 0.7 |
| | IGHV 3S55 | 2 | 0.7 |
| | IGHV 3S56 | 3 | 1.1 |
| | IGHV 3S60 | 6 | 2.2 |
| | IGHV 3S61 | 3 | 1.1 |
| | IGHV 3S65 | 16 | 6.0 |
| | IGHV 3S67 | 1 | 0.4 |
| | Other | 14 | 5.2 |
| | IGHV 3S10/3S6/3S9 | 1 | 0.4 |
| | IGHV 3S30/3S31 | 3 | 1.1 |
| | IGHV 3S36/3S53 | 1 | 0.4 |
| | IGHV 3S39/3S42 | 1 | 0.4 |
| | IGHV 3S53/3S54/3S56/3S57 | 8 | 3.0 |
| | IGHD 1 | 26 | 9.7 |
| | IGHD 2 | 28 | 10.5 |
| | IGHD 3 | 41 | 15.4 |
| | IGHD 4 | 16 | 6.0 |
| | IGHD 5 | 66 | 24.7 |
| | IGHD 6 | 34 | 12.7 |
| | IGHD 7 | 8 | 3.0 |
| | IGHD 8 | 2 | 0.7 |
| | n.d. | 46 | 17.2 |
| | IGHJ 2 | 3 | 1.1 |
| | IGHJ 3 | 11 | 4.1 |
| | IGHJ 4 | 100 | 37.5 |
| | IGHJ 4/6 | 14 | 5.2 |
| | IGHJ 6 | 118 | 44.2 |
| | IGHJ 7 | 20 | 7.5 |
| | no rearrangement | 1 | 0.4 |

*n.d. No result in IGMT analysis

In general, sequence redundancy was used to guide clone selection for further study. For instance, the screen for anti-GTD nanobodies in the TcdA library yielded 30 identical clones. A representative of this clade, A1A6, was selected for further study. Of the 25 TcdA clones selected for study, 16 were recovered multiple times (FIG. 5A). Similarly, 26 identical GTD directed clones were found within the TcdB panel and 11 of the 17 TcdB clones selected for additional study were identified more than once (FIG. 5B). Following promising results in neutralization studies, described below, the TcdA anti-APD-DD panel was expanded such that a total of 18 clones in this group were characterized. The highlighted antibodies (FIG. 5) were all expressed and purified to homogeneity.

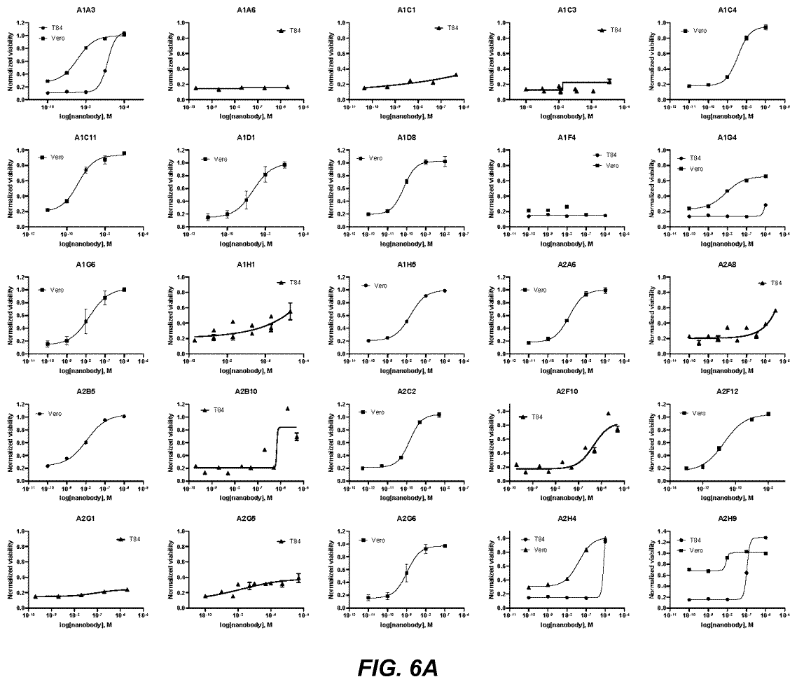

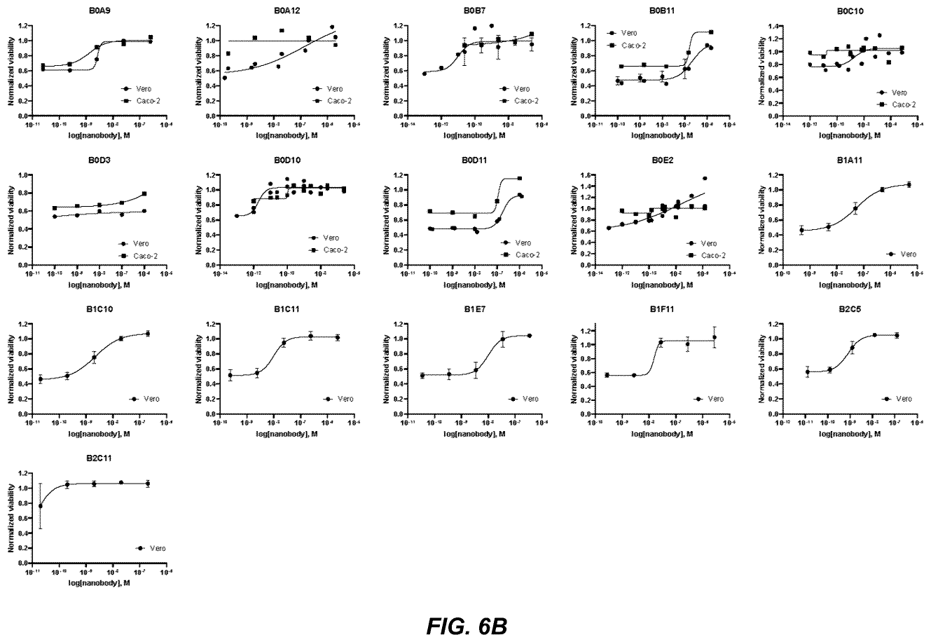

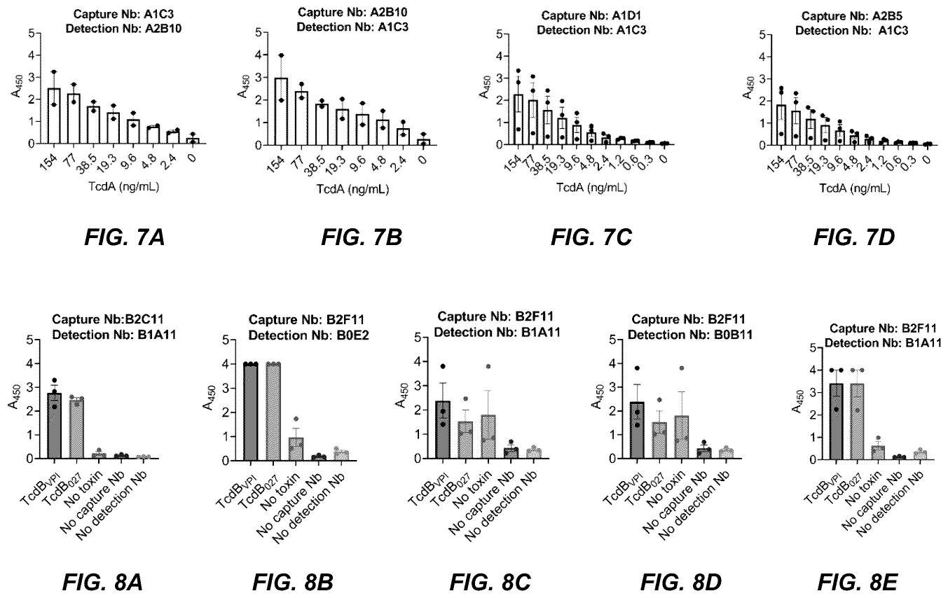

Toxin neutralization experiments were performed using purified toxin and purified nanobodies in a cell viability assay. For TcdA, nanobody-dependent neutralization was assessed using T84 and Vero cells. For TcdB, Caco-2 and Vero cells were used. Neutralization data are summarized in Table 6, and viability curves are shown in FIG. 6. Despite the functionally similar domains, the neutralization data suggest significant differences in how the two toxins can be inhibited. Within TcdA clones, strong neutralizing activity was seen only against the putative APD-DD targeted clones with 15 of 18 nanobodies able to neutralize and 5 of these clones showing sub-nanomolar neutralization. The GTD-targeted clone, A1A6, showed no neutralizing activity. In contrast, all but one of the GTD-targeted TcdB nanobodies were neutralizing, with multiple potent neutralizers within the group. Similarly, only weak neutralizers were identified against the TcdA CROPS R6R7 region despite potent neutralizers being found against the TcdB CROPS.

TABLE 6

EC50 (nM)

| Nanobody | TcdA Domain | T84 | Vero |
|---|---|---|---|
| A1A6 | GTD | n.d. | n.t. |
| A2F12 | APD-DD | 1.1 | <1 |
| A1D8 | APD-DD | 2.1 | <1 |
| A2C2 | APD-DD | 2.4 | <1 |
| A2G6 | APD-DD | 9.2 | <1 |
| A1C11 | APD-DD | 10.4 | <1 |
| A1D1 | APD-DD | n.t. | 1.5 |
| A1G6 | APD-DD | n.t. | 6.4 |
| A1H5 | APD-DD | n.t. | 15.7 |
| A2B5 | APD-DD | n.t. | 11.1 |
| A1C4 | APD-DD | 60.3 | 2.5 |
| A2A6 | APD-DD | 91 | 1 |
| A2H9 | APD-DD | 105.3 | 8.7 |
| A1A3 | APD-DD | 135.5 | 3.7 |
| A2H4 | APD-DD | 838.3 | 40.9 |
| A1G4 | APD-DD | 843.7 | 8.5 |
| A2G5 | APD-DD | >10 μM | n.t. |
| A1F4 | APD-DD | >10 μM | n.d. |
| A1C3 | APD-DD | n.d. | n.t. |
| A2F10 | CROPs-R6R7 | 455.2 | n.t. |
| A2B10 | CROPs-R6R7 | 677.5 | n.t. |
| A1C1 | CROPs-R6R7 | >10 μM | n.t. |
| A1H1 | CROPs-R6R7 | >10 μM | n.t |
| A2A8 | CROPs-R6R7 | >10 μM | n.t. |
| A2G1 | CROPs-R6R7 | n.d. | n.t. |
| | | Caco-2 | Vero |
| B1C10 | GTD | n.t. | 2.2 |
| B1E7 | GTD | n.t. | 10 |
| B0D11 | GTD | 110.9 | 176 |
| B0B11 | GTD | 160.7 | 350 |
| B0B7 | GTD | n.d. | <1 |
| B2C5 | GTD | n.t. | <1 |
| B2C11 | GTD | n.t. | <1 |
| B0D3 | GTD | n.d. | n.d. |
| B0C10 | DD | <1 | 1.3 |
| B1C11 | DD | n.t. | 10 |
| B0A9 | DD | 1.3 | 12.9 |
| B0E2 | DD | <1 | 28 |
| B0A12 | DD | n.d. | 141 |
| B0D10 | DD | <1 | <1 |
| B2F11 | CROPs | n.t. | 15.6 |
| B1A11 | CROPs | n.t. | 38.4 |

| | TcdA | TcdB |
|---|---|---|
| Tested for neutralization | 25 | 16 |
| Neutralizers (EC50 < 1 μM) | 17 | 15 |
| Specificity of neutralizers | | |
| GTD | 0 | 7 |
| APD-DD or DD | 15 | 6 |
| CROPs | 2 | 2 |

n.d. = not detected within tested range
n.t. = not tested

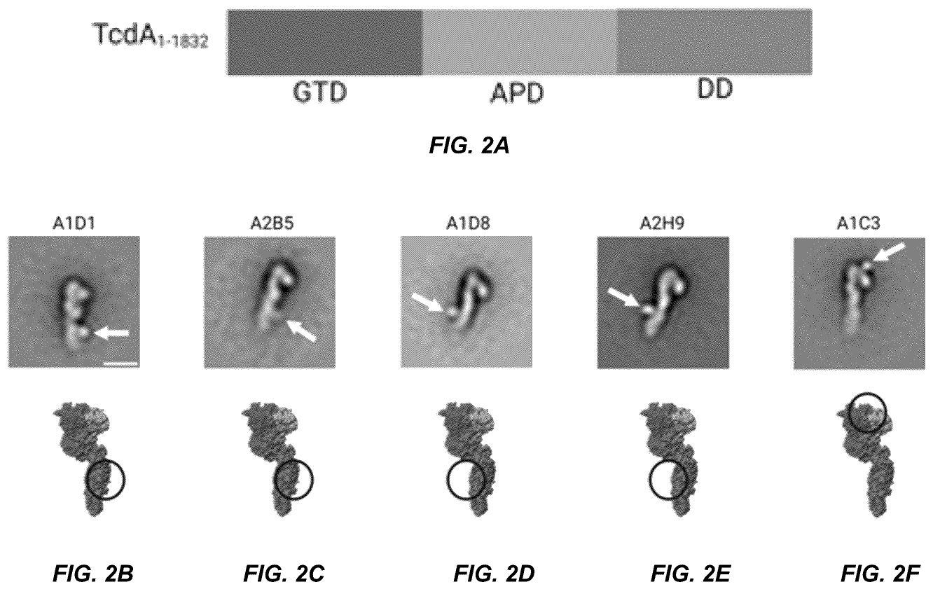

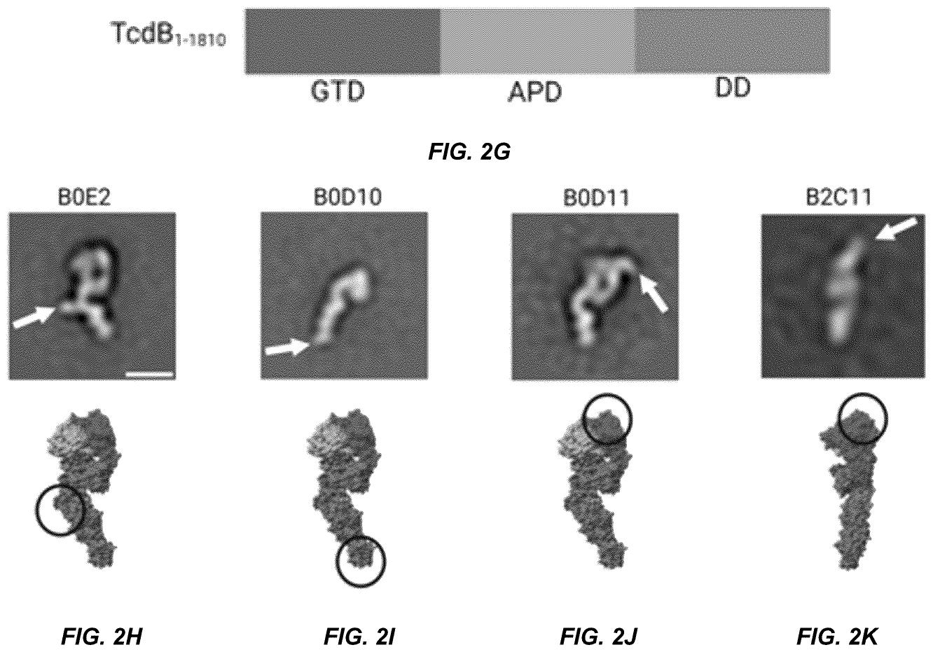

Negative stain electron microscopy (EM) was performed with single particle averaging for a subset of nanobodies to confirm binding to toxin and to locate the binding site of each nanobody (FIG. 2). We were particularly interested in the nanobodies where we had deduced binding to the TcdA APD-DD. The A1D1, A1H5, A1D8, and A2H9 nanobodies all bind the DD, and all neutralize (FIGS. 2 and 6 and Table 3). A1C3, a non-neutralizing TcdA clone, binds near the interface of the GTD, APD, and DD (FIG. 2). As with TcdA, we focused on locating the binding site of a subset of TcdB nanobodies. B0E2 and B0D10 bound the DD, and both neutralized (FIGS. 2 and 6 and Table 3). Both B0D11 and B2C11 bound the GTD; however, only B0D11 could neutralize (FIGS. 2 and 6 and Table 3).

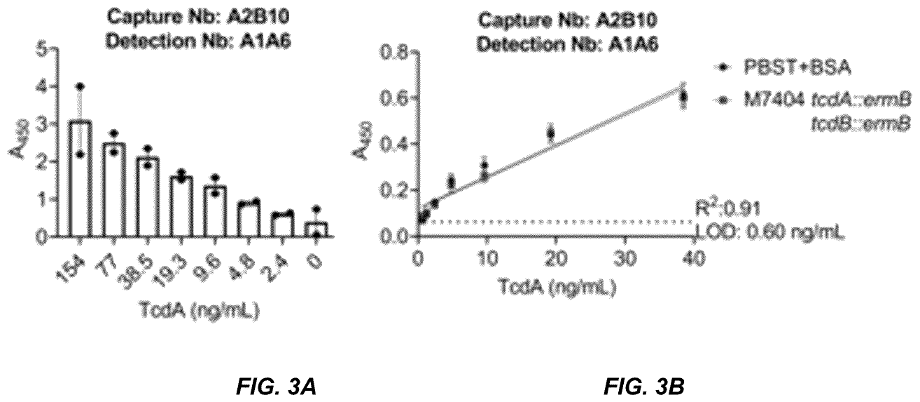

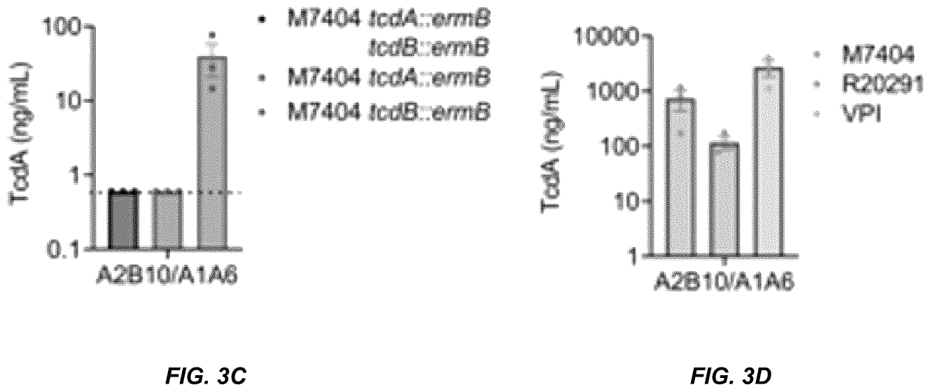

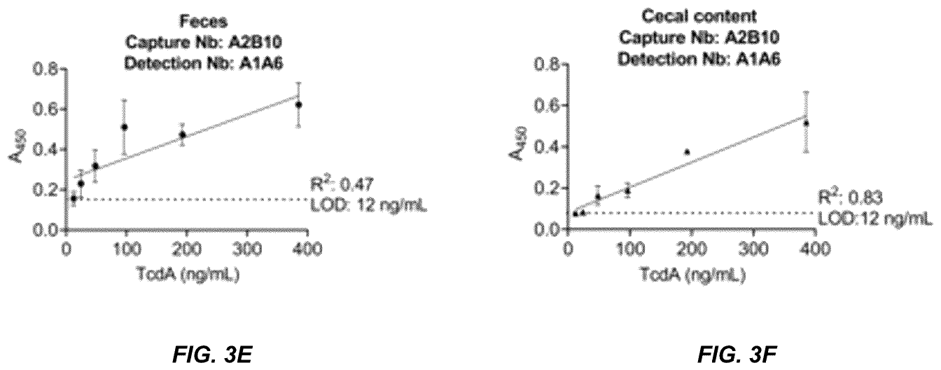

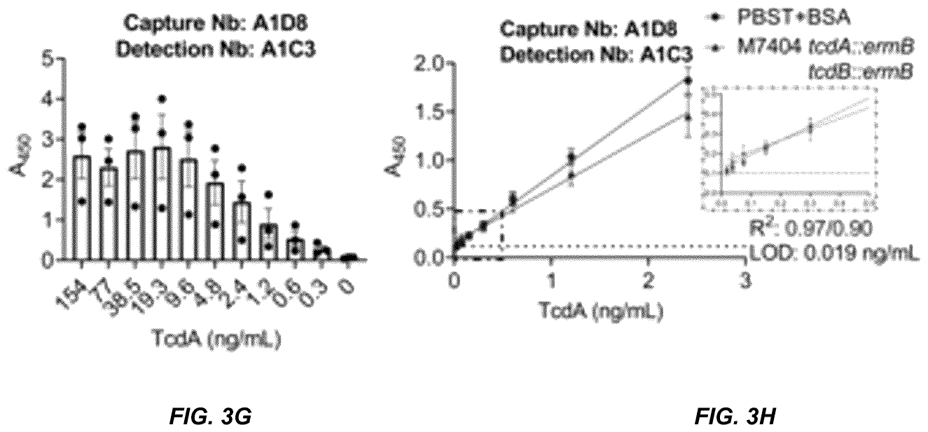

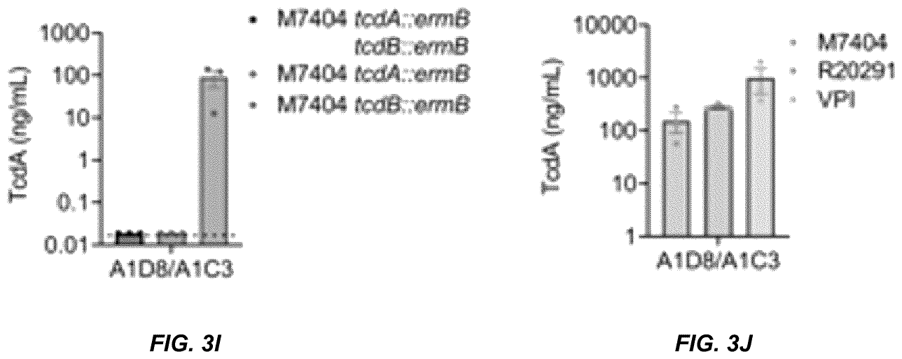

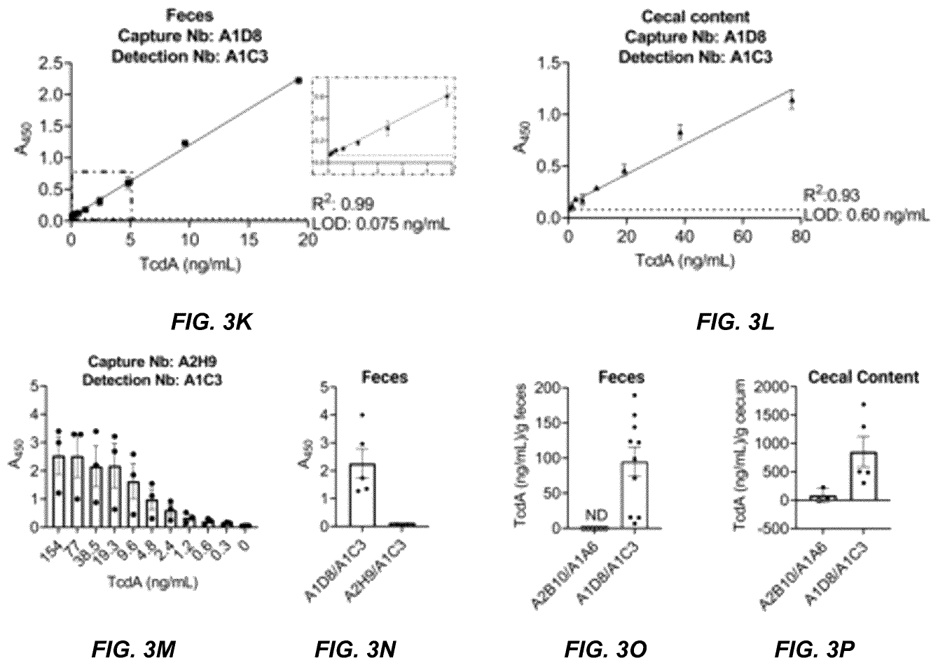

With this understanding of where nanobodies were binding, we chose to develop sandwich ELISA-based quantification assays for TcdA and TcdB. In all assays, plates were coated with a capture nanobody, and we biotinylated the detection nanobody to enable use of a streptavidin-horseradish peroxidase (HRP) assay for the readout. For TcdA, a CROPS targeted capture nanobody, A2B10, was used in conjunction with the GTD targeted detection nanobody, A1A6 (FIG. 3, panels A-F). This pairing was effective at detecting TcdA in buffer or culture supernatant (from a toxin deleted strain), showing a limit of detection (LOD) of 0.6 ng/ml. The pairing was 20-fold worse at detecting toxin added to resuspended mouse feces or mouse cecal content (FIGS. 3A, 3B, 3E, and 3F). To address this limitation, we then developed an assay using A1D8 and A1C3 as capture and detection nanobodies, respectively. Both clones bind the APD-DD region but bind to distinctly different regions (FIG. 2). This assay was significantly better, with a LOD of 0.19 ng/mL in buffer or culture supernatant, 0.075 ng/ml in resuspended feces, and 0.6 ng/ml in cecal content (FIGS. 3G, 3H, 3K, 3L). Both capture and detection pairs were able to detect TcdA from strains M7404, R20291, and VPI10463 (FIG. 3, panels D and J). TcdA was then quantified in fecal and cecal samples from C. difficile infected mice two days after infection. Panels N and O show that the A1D8/A1C3 pair was effective while the A2H9/A1C3, A2B10/A1C3, and A2B10/A1A6 pairs were not. The differences between the ability of pairs of nanobodies to detect TcdA motivated us to test additional pairs, and to swap capture and detection nanobodies. FIG. 6 shows results from toxin titrations using capture/detection pairs A1C3/A2B10, A2B10/A1C3, A1D1/A1C3, and A2B5/A1C3. These pairs all work in vitro.

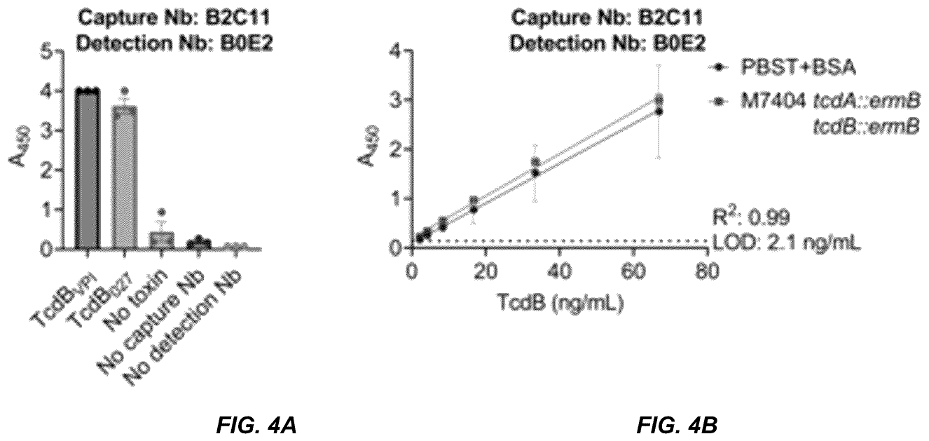

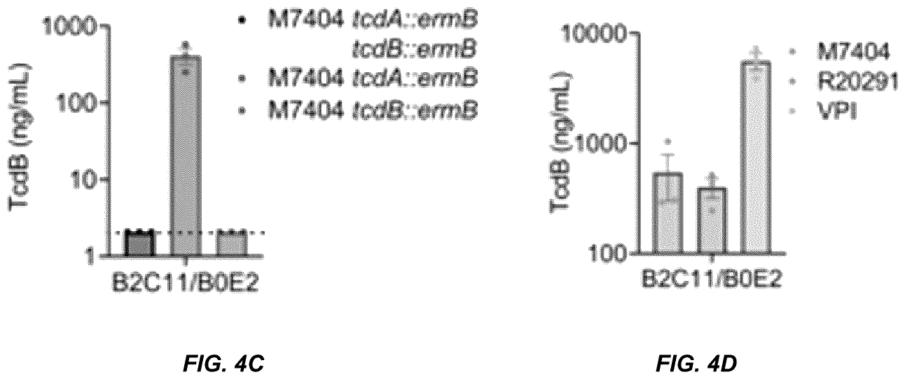

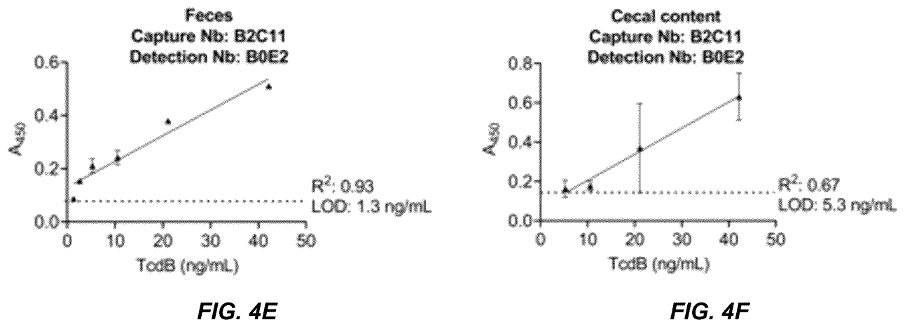

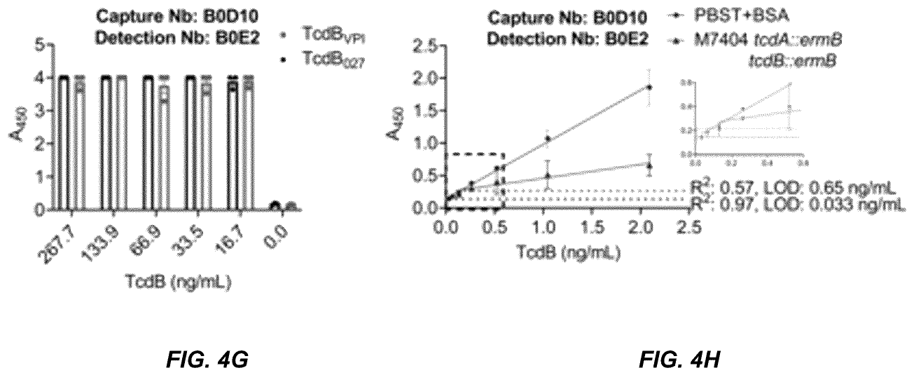

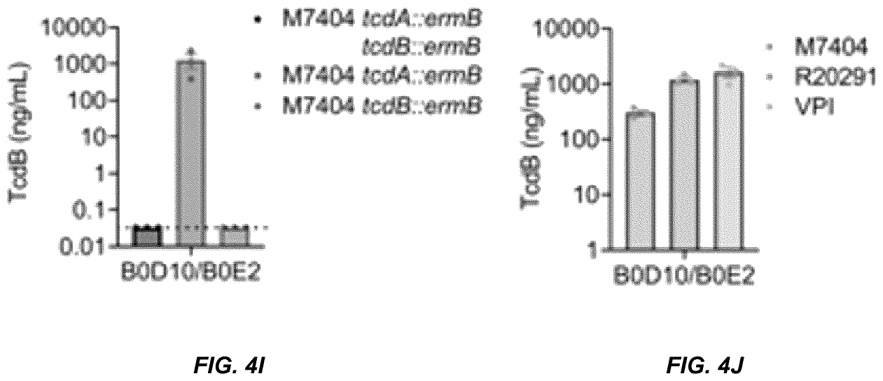

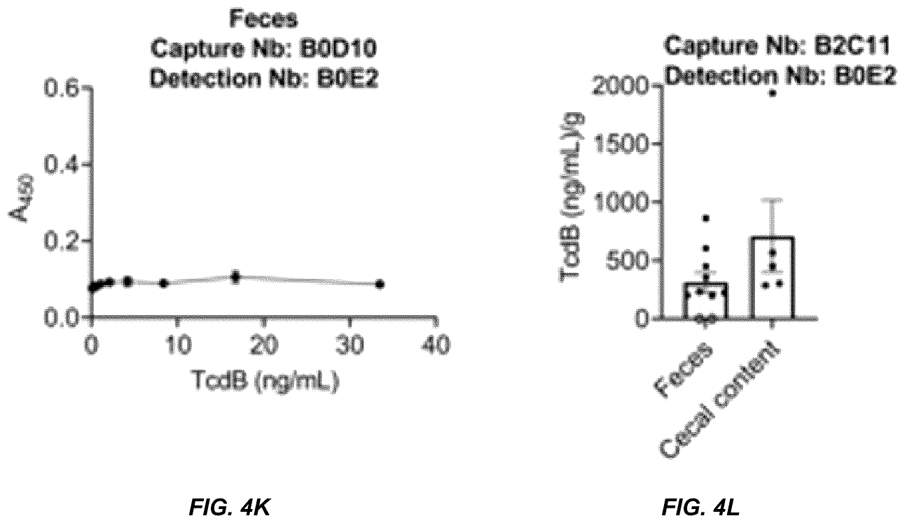

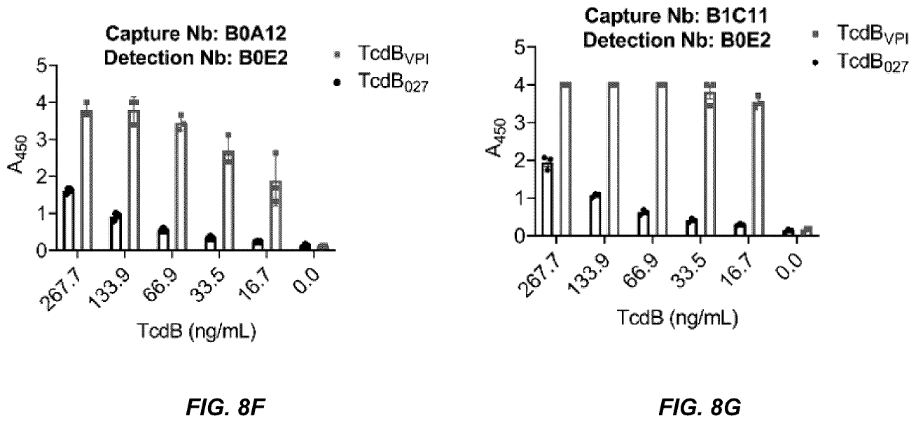

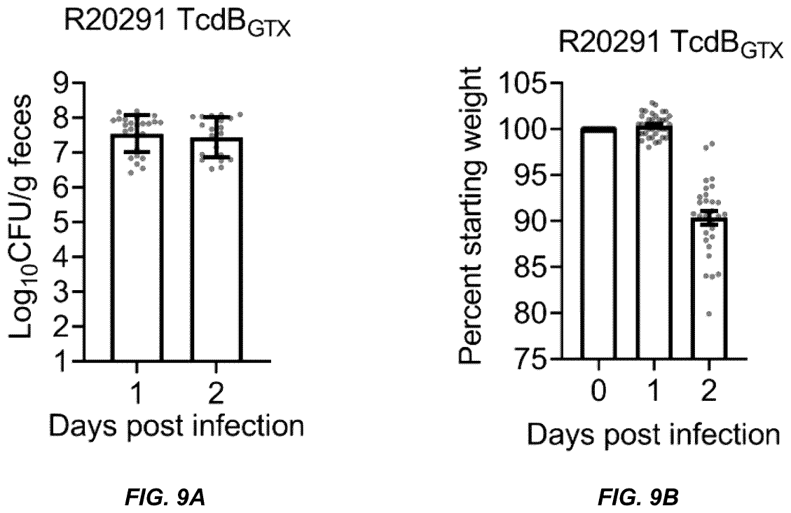

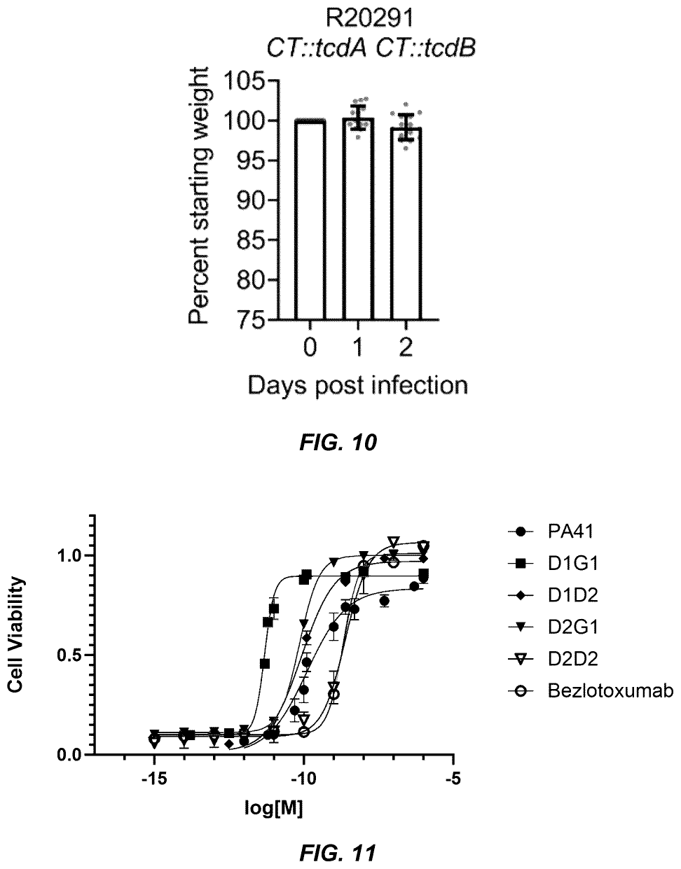

A similar approach was used to develop a sandwich ELISA for TcdB (FIG. 4). B2C11 and B0D10 were used as capture nanobodies, and B0E2 was biotinylated and used for detection. TcdB sequences vary across strains and can be classified into 5 different sequence types. TcdB_{VPI} was derived from a VPI10463 strain and is a member of the TcdB1 family of sequences, while TcdB₀₂₇ can be found in B1/NAP1/027 strains such as R20291 and M7404 and is a member of the TcdB2 family. Both pairs were effective in recognizing TcdB1and TcdB2sequences (FIG. 4A, 4G) The B2C11/B0E2 pair had a LOD of 2.1 ng/ml in buffer and culture supernatant while the B0D10/B0E2 pair had a LOD of 0.57 ng/ml in buffer and 0.033 ng/ml in culture supernatant (FIG. 4B, 4H). Both pairs could detect TcdB from strains M7404, R20291, and VPI10463 (FIGS. 4D and 4J). Despite these similarities, the B2C11/B0E2 was moderately effective in detecting TcdB spiked into the feces and cecal contents of mice while the B0D10/B0E2 pair was completely unable to detect toxin in resuspended feces (FIG. 4K). Therefore, the B2C11/B0E2 based ELISA was used to quantify TcdB in fecal and cecal samples from C. difficile infected mice two days after infection (FIG. 4L). We tested an additional panel of capture/detection pairs for the ability to recognize TcdB from VPI10463 or R20291 (FIG. 8) showing that the B2C11/B1A11, B2F11/B0E2, B2F11/B1A11, B0A12/B0E2, and B1C11/B0E2pairs worked well while the B2F11/B1A11 and B2F11/B0B11 pairs did not.

### Discussion

In this study, non-toxic point mutants of TcdA and TcdB were used without further inactivation, such as crosslinking or boiling, to immunize alpacas. We screened the nanobodies from TcdA and TcdB-specific panning steps for binding to defined toxin domains, resulting in panels of domain specific nanobodies. Prior studies have focused on the CROPS domain, in part because it was thought to contain the receptor-binding function and in part because it was thought to be immunodominant (Yang Z, et al. J Infect Dis. 2014 210:964-972; Castagliuolo I, et al. Infect Immun. 2004 72:2827-2836). The data summarized in Table 1 suggest that no domain is immunodominant. This observation holds both when considering unique and total clones. A key difference between our study and the prior study where alpacas were immunized with full-length TcdA and TcdB mutants is that we did more rounds of immunization (8 versus ≤5) with shorter spacing between immunizations (2 weeks versus ≥3 weeks). These subtle differences may have promoted a more diverse immune response.

Despite broad domain coverage, both the TcdA and TcdB nanobody panels were very constrained in variable gene usage (Table 2). The dominant genes used IGHV 3-3 for TcdA and IGHV 3S53 for TcdB and are known to be among the most commonly used alpaca variable regions. However, they normally account for ˜19% and ˜15% of the total V_{HH} repertoire

[30], suggesting that a broad immune response in terms of epitopes targeted can arise from very restricted gene usage.

Neutralization studies were focused on nanobodies that bound outside the CROPS region, both because we had many such clones and because CROPS targeted nanobodies and antibodies have been extensively studied (Hussack G, et al. J Biol Chem. 2011 286:8961-8976; Orth P, et al. J Biol Chem. 2014 289:18008-18021; Mileto S J, et al. Gut Microbes. 2022 14:2117504). Despite this focus, we did find that TcdB CROPS binding nanobodies were neutralizing. While the mechanism for this neutralization is currently unknown, it could suggest a role for CROPS-dependent glycan binding or as yet unidentified protein receptors in TcdB interactions with host cells. The DD, however, was the target of all potent TcdA binding clones. This was also unexpected and a better understanding of the mechanism of inhibition used by these nanobodies merits further investigation. There is precedent, in the case of the well-studied clinical candidate PA41, for neutralizing antibodies to block GTD delivery. However in that instance GTD is blocked from leaving the endosome by direct binding of antibody PA41 to the GTD (Kroh HK, et al. J Biol Chem. 2018 293:941-952). Whatever the mechanism by which DD domain nanobodies neutralize, they likely target a conserved intoxication step because potent neutralizers binding the DD were found for TcdA and TcdB. Upon recognizing that potent neutralizers bound the TcdA DD, we expanded the panel of clones against this domain.

In addition to our interest in identifying neutralizers, we speculated that nanobodies recognizing discrete epitopes on the surface of the DD might improve the efficacy of a sandwich ELISA assay. In addition to autoprocessing and release of the GTD, the toxins are subjected to exogenous proteases withing the complex milieu of the intestinal tract. Nanobody reagents that bind to N-or C-terminal sequences of the toxins have the potential to lose efficacy as the toxins undergo proteolytic degradation. Indeed, several nanobody pairs were identified that were highly sensitive in ELISA reagents in PBS or media, but ineffective in the feces or cecal contents of mice. Efforts to focus on nanobodies binding unique areas of the DD was an effort to mitigate that issue. For example, while CROPS directed capture and GTD directed detection (A2B10/A1A6) worked well for quantifying TcdA in buffer, it did not work well in feces or cecal content. The switch to two nanobodies binding distinct epitopes on the TcdA DD, A1D8and A1C3, led to a marked improvement in our ability to detect TcdA in the cecal and fecal contents of mice. However, adopting this strategy for TcdB did not result in the same improvements. B0D10 and B0E2, which target different sites on the TcdB DD, were a very efficient ELISA pair in buffer or culture supernatant but were completely unable to detect TcdB in feces. This loss in efficacy may result not from proteolytic degradation, but rather from an occlusion or conformational disruption of the B0D10 nanobody epitope when the toxin is in a complex mixture. B2C11, a GTD targeted clone, can pair with B0E2 to efficiently measure toxin in feces despite having a higher LOD in buffer or media. If true that binding sites are being disrupted by a conformational change or occluded through carbohydrate, protein, or lipid binding, or other non-specific mechanisms such as aggregation, the process of identifying effective targets for in vivo neutralization may need to also consider toxin epitope availability within the complex of environment of the intestinal lumen.

In summary, a panel of nanobodies that bind specific structural and functional domains of TcdA and TcdB have been developed. In testing for neutralization, it was noted that many of the potent neutralizers of TcdA bind epitopes within the delivery domain, a finding that could reflect roles of the delivery domain in receptor binding and/or the conserved role of pore-formation in the delivery of the toxin enzyme domains to the cytosol. Potent neutralizers against TcdB target the GTD, DD, or CROPS domains. Exploring the therapeutic potential of these reagents will require on-going development. Nanobodies lack Fc functions and have short half-lives in vivo but there is precedent for therapeutic and preventative development by expressing the nanobodies as chimeric fusions and through probiotic delivery strategies (Chen K, et al. Science Translational Medicine. 2020 12).

The nanobodies were also used for the creation of sandwich ELISA assays. While quantitation was possible by comparison with standard curves in vitro, it was noted that a decrease in efficacy when quantifying toxins in the cecal or fecal contents of mice. This could reflect toxin proteolysis and degradation or the occlusion of antibody binding sites in complex mixtures. As such, the assays are not robust for absolute quantitation or clinical diagnostics in vivo, but rather, are comparative tools that should allow researchers to monitor the dynamics of TcdA and TcdB production over time, and the impact of various experimental interventions on toxin production in vivo.

### MATERIAL AND METHODS

### Isolation of Anti-TcdA and TcdB-Nanobodies:

TcdA and TcdB-targeted nanobodies were developed in collaboration with Turkey Creek Biotech, Waverly, Tennessee, USA and the Vanderbilt Antibody and Protein Resource core facility (VAPR), Vanderbilt University, Nashville, TN. The strategy was similar to other established protocols (Maass D R, et al. J Immunol Methods. 2007 324:13-25; Pardon E, et al. Nat Protoc. 2014 9:674-693). An alpaca was immunized 6 times with 125 μg of purified mutant toxins in Gerbu adjuvant. The TcdA used was an in inactive mutant, a mutation to the DXD motif in the glucosyltransferase domain. The TcdB used was an L1106K mutant. Following immunization, blood was drawn into citrate containing blood bags, and peripheral blood mononuclear cells (PBMCs) were isolated by centrifugation from ˜100 mL of blood using Sepmate centrifugal devices (Stemcell Technologies). A cDNA library was made by reverse transcription using oligo dT primers and Superscript IV reverse transcriptase (ThermoScientific). A two-step, nested, PCR strategy was used to amplify coding regions of VHH fragments. This was done as described (Maass D R, et al. J Immunol Methods. 2007 324:13-25; Pardon E, et al. Nat Protoc. 2014 9:674-693). The resulting PCR fragments were ligated into pBBBR3, a modified pADL22 vector (Antibody Design Labs), containing sequences for in-frame, C-terminal, HA and hexahistadine tags. The plasmids were electroporated into high-efficiency TG1 cells (Lucent), and phage were produced using CM13 helper phage.

A single round of panning was done against 10 μg TcdA GTX or TcdB L1106K immobilized on maxisorb plates. Three wells of a maxisorb plate were coated overnight with 10 μg of toxin in PBS with three PBS coated wells serving as controls. Wells were blocked with 2% nonfat milk in PBS, washed, and incubated with 2×10¹¹ phage particles in blocking buffer for 1 hour. After extensive alternating washes with PBS and PBS +0.5% Tween 20, phage were eluted with 100 μL 100 mM Glycine pH 2.2, which was immediately neutralized with 1M pH 8 Tris. Recovered phage were used to infect TG1 E. coli and single clones were picked into deep well 96 blocks or terrific broth with 100 μg/mL ampicillin. Plates were grown at 37° C. for 5 hours followed by 28° C. overnight. Bacteria were pelleted and lysed my two freeze thaw cycles with a total of 400 μL of phosphate buffered saline (PBS pH 7.4). Positive clones were identified with a modified ELISA using a maxisorb plate coated with 0.5 μg of toxin per well, which was detected with 25-50 μL of periplasmic extract. The plate was developed using an anti-HA (12CA5) antibody followed by HRP-labeled goat anti-mouse secondary (Jackson ImmunoResearch) and 1 Step Ultra-ELISA TMB substrate (ThermoFisher).

### Nanobody Expression & Purification

Selected nanobody constructs were expressed and purified from either T7 shuffle E. coli (New England Biolabs) or expi283 cells (ThermoFisher). VHH genes were codon optimized, synthesized, and sub-cloned into pET28a or pcDNA3.4 plasmids (Genscript). For bacterial expression, an overnight culture of T7Shuffle transformed with a nanobody in pET28a was diluted 1:100 into fresh Luria Broth with 50 μg/mL kanamycin and grown at 37° C. until a OD600 of ˜1.2 was reached. The temperature was lowered to 18° C., 1 mM IPTG was added, and protein expression was continued overnight. Cells were harvested by centrifugation and lysed with an emulsiflex. Nanobody was purified using Talon affinity resin (Takara). Clones that did not express well in E. coli were expressed from pcDNA3.4 in expi293 cells following the manufacturers protocol (ThermoScientific) except using Freestyle F17 media and PEI as a transfection reagent (Boussif O, et al. Proc Natl Acad Sci USA. 1995 92:7297-7301). Nanobodies were purified from clarified tissue culture supernatants using Talon affinity resin (Takara). Eluted nanobodies were concentrated in an Amicon centrifugal spin concentrator (3K MWCO), then run over an S-75 size exclusion column equilibrated in PBS pH 7.4. Fractions were analyzed by SDS-PAGE, pooled, snap-frozen in liquid N2, and stored at −70° C. until use.

Selected nanobodies with C-terminal Avi-tags were biotinylated as described [38]. Briefly, a sample of Avi-tagged nanobody (100 mM) was incubated with 10 mM MgCl₂, 10 mM ATP, 1/10^{th} mass BirA (Addgene plasmid 20857), and 50 M D-biotin at 30° C. for 1 h Samples were further purified over an S-75 size exclusion column equilibrated in PBS pH 7.4. Fractions containing biotinylated nanobody were analyzed by SDS-PAGE, and nanobody-containing fractions (>90% purity) were stored at −20° C.

### Bacterial Growth Conditions, Medium, and Strains

C. difficile strains were grown on BHIS (brain heart infusion-supplemented) medium or TY medium in a strict anaerobic environment within a COY anaerobic chamber (5% H₂, 5% N₂, and 90% CO₂). E. coli strains were maintained on Lysogeny Broth (LB) supplemented with respective antibiotics. All bacterial strains can be found in Table 7 and plasmids in this study can be found in Table 8.

TABLE 7
Strains used in this study

| | Strain name | |
|---|---|---|
| Number | C. difficile | Relevant characteristics |
| DBLCD6 | R20291 | Wild-type BI/NAPI/027 from Nottingham Clostridia Research |
| | | Group |
| DBLCD1 | R20291 | DBLCD6 containing two ClosTron insertions one after |
| | tcdA1584s::CT | nucleotide 1584 of tedA harboring ermB (Ermr) and another |
| | tcdB1578s::CT | after nucleotide 1578 of tcdB harboring catP (Camr). |
| DBLCD5 | R20291- | DBLCD6 containing TcdB::D286N/D288N |
| | TcdBGTX | |
| DBLCD7 | VPI 10463 | Abdominal wound isolate Toxinotype 0 |
| DBLCD24 | M7404 | Canadian B1/NAP1/027 isolate |
| DBLCD62 | M7404 | DBLCD24 containing targetron insertion after nucleotide |
| | tcdA4068s::ermB | 4068 of tcdA harboring ermB (Ermr). |
| DBLCD64 | M7404 | DBLCD24 containing targetron insertion after nucleotide |
| | tcdB1587s::ermB | 1587 of tcdB harboring ermB (Ermr). |
| DBLCD66 | M7404 | DBLCD64 containing targetron insertion after nucleotide |
| | | 4068 of tcdA harboring ermB (Ermr). |

TABLE 8
Plasmids used in this study

| Number | Plasmid | Relevant characteristics |
|---|---|---|
| | Parent Plasmids | |
| | pC-His1622 | Low copy plasmid for protein overexpression in Bacillus |
| | | megaterium, C-terminal 6X-histidine tag, inducible P_{xylR}, |
| | | Tet^{r}, Amp^{r} |
| | pET28a/b/c(+) | Low copy plasmid for protein overexpression, N- and C- |
| | | terminal 6X-histidine tag, inducible with lacI, Kan^{r} |
| | pBG101 | Low copy plasmid for protein overexpression, pET27 |
| | | derivative, N-terminal 6X-histidine tag, inducible with lacI, |
| | | Kan^{r} |
| | pBBR3 | Modified pADL22 plasmid, C-terminal HA-tag and 6X- |
| | | histidine tag, inducible with lacI, Amp^{r} |
| | Toxin Constructs | |
| | TcdA | |
| pBL282 | pC-His1622-tcdA | pC-HIS1622, TcdA₁₋₂₇₁₀ |
| pBL764 | pC-His1622- | pC-HIS1622, TcdA₁₋₂₇₁₀ with mutations in the |
| | tcdA_{D285N/D287N} | glucosyltransferase domain (D285N, D287N) |
| pBL515 | pC-His1622- | pC-HIS1622, TcdA₁₋₁₈₃₂ (no CROPs domain) |
| | tcdA₁₋₁₈₃₂ | |
| pBL657 | pC-His1622- | pC-HIS1622, TcdA₁₋₁₈₀₉ (no CROPs domain) |
| | tcdA₁₋₁₈₀₉ | |
| pBL500 | pC-His1622-tcdA₁₋₅₄₂ | pC-HIS1622, TcdA₁₋₅₄₂ (glucosyltransferase domain only) |
| pBL840 | pET28a(+)- | pET28a(+), TcdA₂₄₆₀₋₂₇₁₀ (CROPs repeats 6-7) |
| | tcdA₂₄₆₀₋₂₇₁₀ | |
| | TcdB | |
| pBL377 | pC-His1622-tcdB | pC-HIS1622, TcdB₁₋₂₃₆₆ |
| pBL682 | pC-His1622- | pC-HIS1622, TcdB₁₋₂₃₆₆ with point mutation L1106K |
| | tcdB_{L1106K} | |
| pBL832 | pC-His1622- | pC-HIS1622, TcdB₁₋₁₈₁₀ (no CROPs domain) |
| | tcdB₁₋₁₈₁₀ | |
| pBL834 | pC-His1622-tcdB₁₋₅₄₃ | pC-HIS1622, TcdB₁₋₅₄₃ (glucosyltransferase domain only) |
| pBL757 | pET28b(+)- | pET28b(+), TcdB₈₄₂₋₁₈₃₄ (delivery domain only) |
| | tcdB₈₄₂₋₁₈₃₄ | |
| pBL281 | pBG101-tcdB₁₈₃₂₋₂₃₆₆ | pBG101, TcdB₁₈₂₇₋₂₃₆₆ (CROPs domain only) |
| | Nanobodies | |
| | TcdA | |
| pNB126 | pET28b(+)-A1A3 | pET28b(+), nanobody from plate A1 location A3 |
| pNB104 | pET28b(+)-A1A6 | pET28b(+), nanobody from plate A1 location A6 |
| pNB105 | pET28a(+)-A1C1 | pET28a(+), nanobody from plate A1 location C1 |
| pNB127 | pBBR3-A1C4 | pBBR3, nanobody from plate A1 location C4 |
| pNB128 | pBBR3-A1C11 | pBBR3, nanobody from plate A1 location C11 |
| pNB129 | pBBR3-A1D1 | pBBR3, nanobody from plate A1 location D1 |
| pNB130 | pBBR3-A1D8 | pBBR3, nanobody from plate A1 location D8 |
| pNB131 | pBBR3-A1F4 | pBBR3, nanobody from plate A1 location F4 |
| pNB132 | pBBR3-A1G4 | pBBR3, nanobody from plate A1 location G4 |
| pNB133 | pBBR3-A1G6 | pBBR3, nanobody from plate A1 location G6 |
| pNB107 | pET28a(+)-A1H1 | pET28a(+), nanobody from plate A1 location H1 |
| pNB134 | pBBR3-A1H5 | pBBR3, nanobody from plate A1 location H5 |
| pNB135 | pBBR3-A2A6 | pBBR3, nanobody from plate A2 location A6 |
| pNB109 | pET28a(+)-A2A8 | pET28a(+), nanobody from plate A2 location A8 |
| pNB110 | pET28a(+)-A2B10 | pET28a(+), nanobody from plate A2 location B10 |
| pNB136 | pBBR3-A2B5 | pBBR3, nanobody from plate A2 location B5 |
| pNB137 | pBBR3-A2C2 | pBBR3, nanobody from plate A2 location C2 |
| pNB111 | pET28a(+)-A2F10 | pET28a(+), nanobody from plate A2 location F10 |
| pNB138 | pBBR3-A2F12 | pBBR3, nanobody from plate A2 location F12 |
| pNB112 | pET28a(+)-A2G1 | pET28a(+), nanobody from plate A2 location G1 |
| pNB113 | pET28a(+)-A2G5 | pET28a(+), nanobody from plate A2 location G5 |
| pNB139 | pBBR3-A2G6 | pBBR3, nanobody from plate A2 location G6 |
| pNB140 | pBBR3-A2H4 | pBBR3, nanobody from plate A2 location H4 |
| pNB141 | pBBR3-A2H9 | pBBR3, nanobody from plate A2 location H9 |
| | TcdB | |
| pNB007 | pBBR3-B0A9 | pBBR3, nanobody from plate B0 location A9 |
| pNB010 | pBBR3-B0A12 | pBBR3, nanobody from plate B0 location A12 |
| pNB017 | pBBR3-B0B7 | pBBR3, nanobody from plate B0 location B7 |
| pNB021 | pBBR3-B0B11 | pBBR3, nanobody from plate B0 location B11 |
| pNB032 | pBBR3-B0C10 | pBBR3, nanobody from plate B0 location C10 |
| pNB037 | pBBR3-B0D3 | pBBR3, nanobody from plate B0 location D3 |
| pNB044 | pBBR3-B0D10 | pBBR3, nanobody from plate B0 location D10 |
| pNB045 | pBBR3-B0D11 | pBBR3, nanobody from plate B0 location D11 |
| pNB048 | pBBR3-B0E2 | pBBR3, nanobody from plate B0 location E2 |
| pNB114 | pET28c(+)-B1A11 | pET28c(+), nanobody from plate B1 location A11 |
| pNB116 | pET28c(+)-B1C10 | pET28c(+), nanobody from plate B1 location C10 |
| pNB117 | pET28c(+)-B1C11 | pET28c(+), nanobody from plate B1 location C11 |
| pNB118 | pET28c(+)-B1E7 | pET28c(+), nanobody from plate B1 location E7 |
| pNB120 | pET28c(+)-B2C5 | pET28c(+), nanobody from plate B2 location C5 |
| pNB122 | pET28c(+)-B2C11 | pET28c(+), nanobody from plate B2 location C11 |
| pNB124 | pET28c(+)-B2F11 | pET28c(+), nanobody from plate B2 location F11 |
| | Avi-tagged | |
| pNB142 | pET28b(+)-A1A6-Avi | pET28b(+), nanobody from plate A1 location A6, C-terminal |
| | | HA-tag, Avi-tag, and 5X histidine tag |
| pNB143 | pET28b(+)-A2B10- | pET28b(+), nanobody from plate A2 location B10, C- |
| | Avi | terminal HA-tag, Avi-tag, and 6X histidine tag |
| pNB146 | pET28b(+)-B0E2-Avi | pET28b(+), nanobody from plate B0 location E2, C-terminal |
| | | HA-tag, Avi-tag, and 6X histidine tag |

C. difficile Toxin Constructs Expression & Purification

All recombinant TcdA and TcdB constructs were derived from the VPI10463 strain sequence and were expressed in Bacillus megaterium and purified as described previously. Parent plasmids include full-length TcdA (pBL282), TcdA₁₋₁₈₃₂ (pBL515), TcdA₁₋₁₈₀₉ (pBL657), TcdA-GTD (pBL500), TcdA-R6R7 (pBL840), full-length TcdB (pBL377), TcdB₁₋₁₈₁₀ (pBL832), TcdB-GTD (pBL834), TcdB₈₄₂₋₁₈₃₄ (pBL757), and TcdB-CROPs (pBL281).

### Toxin Domain Specificity Assignment

Domain specificities for individual nanobody clones were determined by ELISA against isolated protein domains (TcdA-GTD, TcdA₁₋₁₈₀₉ (APD-GTD-DD), TcdA-CROPs-R6R7, TcdB-GTD, TcdB₈₄₂₋₁₈₃₄ (delivery domain), TcdB-CROPs. Briefly, protein domains were biotinylated and added to 96-well plates coated with NeutrAvidin (10 μg/mL solution). Bacterial supernatants from TG1 E. coli expressing the individual nanobodies were then added to the wells, testing each 96-well plate against each antigen domain. Bound nanobodies were detected with a Li-Cor 880-labeled mouse anti-HA-tag antibody on a Li-Cor Odyssey imager.

Nanobody Sequence analysis

The DNA plasmids containing the nanobody clones were sequenced (Azenta). The corresponding amino acid sequences for the clones were manually analyzed to confirm the absence of frameshifts and spurious stop codons. Alignments of the full amino acid sequences were made in CLUSTAL OMEGA, and phylogenetic analysis was performed with RAxML using the raxmlGUI platform (Edler D, et al. Methods in Ecology and Evolution. 2021 12:373-377). Results were visualized in ITOL v6.6 (Madeira F, et al. Nucleic Acids Res. 2022 50:W276-W279; Letunic I, et al. Nucleic Acids Res. 2021 49: W293-W296). VHH germline gene analysis was performed with IMGT/HighV-QUEST against the Vicugna pacos (alpaca) reference directory, IGH gene locus (Alamyar E, et al. Immunome Res. 2012 08).

### In Vitro Toxin Neutralization Assays

Multiple cell lines were used to evaluate neutralization of the toxins by individual nanobodies: T84 (TcdA), Caco-2 (TcdB), and Vero (TcdA, TcdB). Cells were plated in 96-well, black, clear-bottom, cell culture plates (Costar) at 3×10³ (T84, Caco-2) or 1.5×10³ (Vero) cells per well and incubated overnight at 37° C. and 5% CO₂. Purified toxins were incubated with serial dilutions of nanobodies for 0.5 hr at room temperature, then added to the cells. Plates were incubated for 72 hr, then the media were aspirated, and fresh media added to cells. CellTiter Blue (Promega) reagent (20 μL/well) was added, and the plates were incubated for 3.5 hr. Fluorescence was read in a Cytation plate reader (Bio-Tek) at 560/590 excitation/emission. Cell viability was determined by subtraction of an untreated control and normalized to the toxin-only value. EC₅₀ was calculated in GraphPad Prism by least squares fit of the log (agonist) vs. response—Variable slope (four parameters) model.

### Negative Stain EM

Purified recombinant TcdA₁₋₁₈₃₂ or TcdB₁₋₁₈₁₀ was run over an Superdex-200 size exclusion column equilibrated in 20 mM Tris, pH 8.0, 100 mM NaCl to remove potential aggregates. The toxins were mixed in a 1:2 molar ratio (100 nM toxin: 200 nM nanobody) with individual purified nanobodies in the same buffer. The proteins were incubated at room temperature for 30 min then diluted four-fold in buffer immediately before application to the grids. Samples (3 μL) were applied to freshly glow-discharged, carbon-coated copper grids (EMS), incubated for 1.5 min at room temperature, and stained for 1.5 min with freshly prepared 0.75% (mass/volume) uranyl formate (Ohi M, et al. Biol Proced Online. 2004 6:23-34). Micrographs were collected at 62,000× magnification (1.7574 Å/pixel) with Serial EM software (Mastronarde D N. J Struct Biol. 2005 152:36-51) on an FEI Tecnai F20 (200 keV) TEM equipped with a Gatan US4000 charge-coupled device camera. Individual particle datasets were picked for each nanobody complex, and two-dimensional alignment and classification was performed in RELION (Scheres S H W. Struct Biol. 2012 180:519-530).

### Anti-Toxin ELISAs

ELISA plates (96-well flat-bottom; Nunc Maxisorp) were coated overnight at 4° C. on an orbital shaker with 100 ng/ml solutions (in PBS) of either A2B10, A1D8 (anti-TcdA) or B2C11, B0D10 (anti-TcdB) capture nanobody. Next, the plates were washed 4 times with PBS+0.05% Tween-20 (PBS-T), and incubated with blocking buffer (PBS-T+2% BSA) for 2 hr at room temperature with shaking, followed by 4 washes with PBS-T. For the toxin standard curves, each toxin was diluted to 1 nM (308 or 270 ng/ml of TcdA/B) in blocking buffer and a 2-fold dilution series was set up. The toxins were added to the coated plate, incubated for 1 hr at room temperature with shaking, then washed 4 times with PBS-T. For the supernatant standard curves, clarified bacterial supernatant was serially diluted (2-fold) and added to the plates. The plates were then incubated for 2 hours at room temperature with shaking and washed 4 times with PBS-T. Captured toxins were detected by addition of 100 μL of solutions containing biotinylated Avi-tagged detection nanobodies: A1A6 (20 ng/ml), A1C3 (5 ng/ml), and B0E2 (5 ng/ml). Plates were incubated for 2 hr at room temperature with shaking and washed 4 times with PBS-T. HRP conjugated Streptavidin (ThermoScientific) was diluted 1:20,000 in blocking buffer, added to the plates, and incubated for 1 hr at room temperature with shaking. Plates were washed five times with PBS-T, then 75 μl of 1 Step UltraTMB ELISA substrate solution (equilibrated to room temperature) was added to each well. For TcdA ELISAs, color development was quenched immediately with 75 μl of 2 M H₂SO₄, and TcdB ELISAs were quenched following a 30 min incubation in the dark. The ELISAs were read at 450 nM in a Cytation plate reader (Biotek). In order to calculate the total amount of toxin captured from the samples, all plates contained a full standard curve.

Toxin Secretion in C. difficile

All strains were streaked onto BHIS with thiamphenicol plates. Well-isolated colonies were picked into TY medium and grown for 16 hr under anaerobic conditions (without shaking). The following day, the resulting growth was sub-cultured 1:200 into fresh TY medium and grown for 24 hr. The cultures were centrifuged at 3000×g for 5 min, then the supernatant was filtered through a 0.8 μM syringe filter and stored at −70° C. until use.

For anti-toxin ELISAs, the supernatants were thawed on ice and serially diluted 2-fold in PBS-T+2% BSA. To account for plate-to-plate variability, rTcdA or rTcdB standard curves were included on each plate, and the total toxin was calculated based on those standard curves. Concentrations of toxin that fell at or below the limit of quantification are plotted on the curve as such.

### Measurement of Toxin Concentrations in a Murine Model

All animal experiments described in this study have been reviewed and approved by the Institutional Animal Care and Use Committee at Vanderbilt University Medical Center. Mice were monitored daily and humanely euthanized via CO₂ asphyxiation at various time points. All animal experiments were performed using the cefoperazone mouse infection model using 10⁴ spores of C. difficile (Theriot C M, et al. Gut Microbes. 2011 2:326-334).

TcdA and TcdB standard curves were generated using mice infected with R20291 tcdA::CT tcdB::CT. Stool was collected daily from mice, and cecal content was harvested at four days post infection. Samples were frozen in liquid Nitrogen and stored in −80° C. Stool and cecal content was homogenized in PBS to a concentration of roughly 50 mg/ml or 500 mg/mL, respectively. ELISAs were performed as described above, except the toxins and fecal or cecal content mixture was incubated at 37° C. for 30 min with shaking followed by 1.5 hrs at room temperature to simulate conditions within the mouse. To account for plate-to-plate variability, rTcdA or rTcdB standard curves were included on each plate.

TcdA and TcdB were measured in mice infected with of R20291 TcdB_{GTX}. Stool and cecal content were collected at two days post infection, and toxin concentrations were determined on the same day. Briefly, fecal and cecal content were resuspended in PBS to a concentration of roughly 50 mg/mL or 500 mg/mL, respectively. A sample of the fecal slurry was plated for CFU's to ensure colonization. The remainder of the slurries were subjected to an ELISA as described above. To account for plate-to-plate variability, rTcdA or rTcdB standard curves were included on each plate. No deviations from the calculated from the standard curve in PBS from the spike in plates compared to the fecal/cecal content measurements was found, therefore the total toxin was calculated based on the standard curves determined from the fecal or cecal content spike-in values. Concentrations of toxin that fell at or below the limit of quantification are plotted on the curve as such.

### Example 2

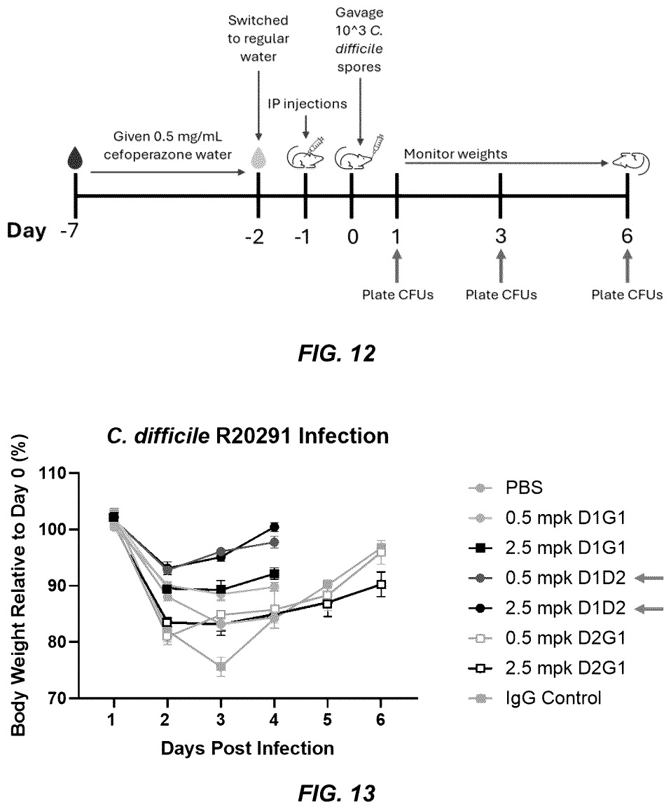

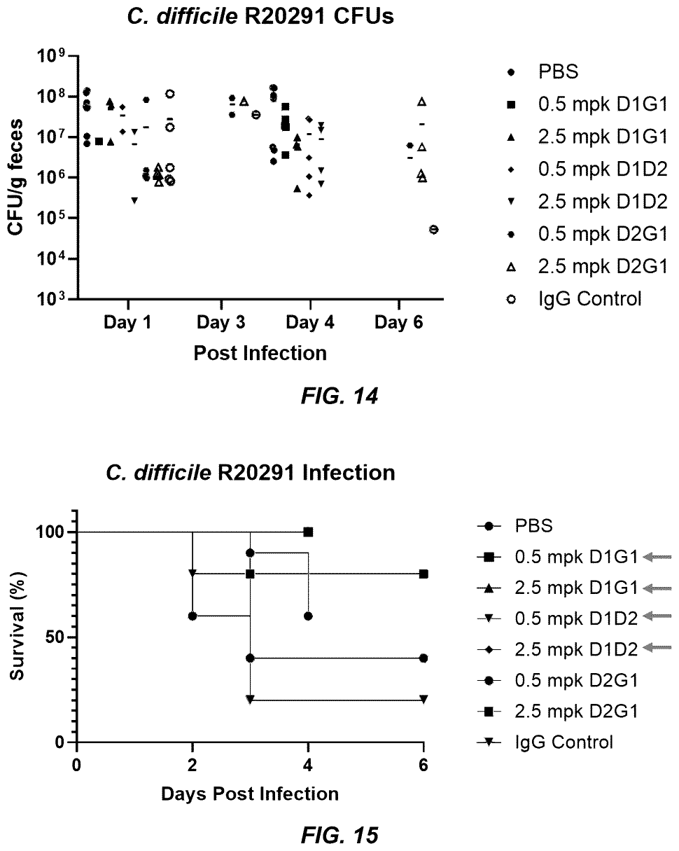

FIG. 11 shows neutralization of TcdB1 on Vero cells. Table 9 provides a summary of IC50 neutralization data. FIG. 12 shows a mouse model of CDI. FIG. 13 shows D1D2 treated mice lose less weight and regain to near pre-infection weight. FIG. 14 shows no clear trends emerged for colony forming units (CFUs) up to day 6 post infection. FIG. 15 shows both D1G1 and D1D2 protect against R20291 infection.

TABLE 9

| | TcdB1 VPI | TcdB2 R20291 | TcdB3 M68 |
|---|---|---|---|
| Bezlotoxumab | 2105 | 4773 | 157 |
| D1G1 (B0E2-B2C11) | 5 | 49 | 8 |
| D1D2 (B0E2-B0D10) | 84 | 631 | 15 |
| D2G1 (B0D10-B2C11) | 72 | 245 | 18 |
| D2D2 (B0D10-B0D10) | 2453 | 2287 | 97 |

D = delivery domain targeting (D1 = B0E2; D2 = B0D10)
G = GTD targeting (G1 = B2C11)

While D1G1 was expected to do the best based on in vitro work, D1D2 outperformed D1G1 in vivo. D1D2 was able to restore weight loss to near pre-infection weight. Although D1D2 performed better than D1G1 for reducing weight loss, both garnered protection in mice against infection

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising a nanobody, wherein the nanobody comprises CDR1, CDR2, and CDR3 sequences,
wherein the CDR1 sequence comprises the amino acid sequence GRIFSIKS (SEQ ID NO:1); wherein the CDR2 comprises the amino acid sequence ITSGGST (SEQ ID NO:2); wherein the CDR3 comprises the amino acid sequence RRVVVTPYPDEYEYDY (SEQ ID NO:3);
wherein the CDR1 sequence comprises the amino acid sequence GFIDDDYA (SEQ ID NO:5); wherein the CDR2 comprises the amino acid sequence ISSSNGKI (SEQ ID NO:6); and wherein the CDR3 comprises the amino acid sequence AAETRGWSYCSGYGWSRYKY (SEQ ID NO:7);
wherein the CDR1 sequence comprises the amino acid sequence GGTFSSYS (SEQ ID NO:9); wherein the CDR2 comprises the amino acid sequence ITWRGIT (SEQ ID NO:10); and wherein the CDR3 comprises the amino acid sequence AARDRRAARIQEFDY (SEQ ID NO:11).
wherein the CDR1 sequence comprises the amino acid sequence GRSFSINT (SEQ ID NO:13); wherein the CDR2 comprises the amino acid sequence ITTGGNT (SEQ ID NO:14); and wherein the CDR3 comprises the amino acid sequence RTVVVTPYPDEFEYDY (SEQ ID NO:15);
wherein the CDR1 sequence comprises the amino acid sequence GRSFSINT (SEQ ID NO:17); wherein the CDR2 comprises the amino acid sequence ITTGGNT (SEQ ID NO:18); and wherein the CDR3 comprises the amino acid sequence RTVVVTPYPDEFEYDY (SEQ ID NO:19);
wherein the CDR1 sequence comprises the amino acid sequence GRTFSTHT (SEQ ID NO:21); wherein the CDR2 comprises the amino acid sequence IRWSDGMT (SEQ ID NO:22); and wherein the CDR3 comprises the amino acid sequence GAGPTMYHPTY (SEQ ID NO:23);
wherein the CDR1 sequence comprises the amino acid sequence GGTFSRYA (SEQ ID NO:24); wherein the CDR2 comprises the amino acid sequence ISYSGATT (SEQ ID NO:25); and wherein the CDR3 comprises the amino acid sequence AAGFFRGDLTKFKLDEYDY (SEQ ID NO:26);
wherein the CDR1 sequence comprises the amino acid sequence GRTFTTYN (SEQ ID NO:28); wherein the CDR2 comprises the amino acid sequence ITGLTRHT (SEQ ID NO:29); and wherein the CDR3 comprises the amino acid sequence AVSSGGDLNERVNYEY (SEQ ID NO:30);
wherein the CDR1 sequence comprises the amino acid sequence GRTLNSYA (SEQ ID NO:32); wherein the CDR2 comprises the amino acid sequence ISRAGGMT (SEQ ID NO:33); and wherein the CDR3 comprises the amino acid sequence AASFALVDSAGAYDY (SEQ ID NO:34);
wherein the CDR1 sequence comprises the amino acid sequence GRAFSSYA (SEQ ID NO:36); wherein the CDR2 comprises the amino acid sequence ISWSGGST (SEQ ID NO:37); and wherein the CDR3 comprises the amino acid sequence AADFSQPLLATVPDDYDY (SEQ ID NO:38);
wherein the CDR1 sequence comprises the amino acid sequence GGTFSKTS (SEQ ID NO:40); wherein the CDR2 comprises the amino acid sequence ITWSGNT (SEQ ID NO:41); and wherein the CDR3 comprises the amino acid sequence AARERTAARIQEFDY (SEQ ID NO:42);
wherein the CDR1 sequence comprises the amino acid sequence GSIFSINA (SEQ ID NO:44); wherein the CDR2 comprises the amino acid sequence ITSGGST (SEQ ID NO:45); and wherein the CDR3 comprises the amino acid sequence HVPWTDDFGWAVKDY (SEQ ID NO:46). In some embodiments, the nanobody has one or more conservative substitutions in SEQ ID NOs: 44, 45, and/or 46.
wherein the CDR1 sequence comprises the amino acid sequence GRTFSRYE (SEQ ID NO:48); wherein the CDR2 comprises the amino acid sequence INRLGRST (SEQ ID NO:49); and wherein the CDR3 comprises the amino acid sequence AAGVRLNLPQIPDVIDF (SEQ ID NO:50);
wherein the CDR1 sequence comprises the amino acid sequence GGTFSSYS (SEQ ID NO:52); wherein the CDR2 comprises the amino acid sequence ITWRNNT (SEQ ID NO:53); and wherein the CDR3 comprises the amino acid sequence AARERRVARIQEFDY (SEQ ID NO:54);
wherein the CDR1 sequence comprises the amino acid sequence GRDFSSGA (SEQ ID NO:56); wherein the CDR2 comprises the amino acid sequence VGWSGGLI (SEQ ID NO:57); and wherein the CDR3 comprises the amino acid sequence AVNSANSCAGYDCHDKPQTYNY (SEQ ID NO:58);
wherein the CDR1 sequence comprises the amino acid sequence ERTFSAYT (SEQ ID NO:60); wherein the CDR2 comprises the amino acid sequence IKWSGSGGIT (SEQ ID NO:61); and wherein the CDR3 comprises the amino acid sequence TAGPTVYNPHY (SEQ ID NO:62);
wherein the CDR1 sequence comprises the amino acid sequence GRTFSRYA (SEQ ID NO:64); wherein the CDR2 comprises the amino acid sequence ISYSGGTT (SEQ ID NO:65); and wherein the CDR3 comprises the amino acid sequence AAGLFRGDLTRFTLDEYDY (SEQ ID NO:66);
wherein the CDR1 sequence comprises the amino acid sequence GRSFSSGA (SEQ ID NO:68); wherein the CDR2 comprises the amino acid sequence VGWSGGLI (SEQ ID NO:69); and wherein the CDR3 comprises the amino acid sequence AINSANSCAGYDCHDKPQVYDY (SEQ ID NO:70);
wherein the CDR1 sequence comprises the amino acid sequence GRTFSRYA (SEQ ID NO:72); wherein the CDR2 comprises the amino acid sequence ISWSGDTT (SEQ ID NO:73); and wherein the CDR3 comprises the amino acid sequence AAGLFRGDLTKFELDEYDY (SEQ ID NO:74);
wherein the CDR1 sequence comprises the amino acid sequence GHSFSTSA (SEQ ID NO:76); wherein the CDR2 comprises the amino acid sequence ISWAGGKI (SEQ ID NO:77); and wherein the CDR3 comprises the amino acid sequence AANSQNMCSGWDCEKQPRVYDF (SEQ ID NO:78);
wherein the CDR1 sequence comprises the amino acid sequence GRTLSSYA (SEQ ID NO:80); wherein the CDR2 comprises the amino acid sequence ISRGGGMT (SEQ ID NO:81); and wherein the CDR3 comprises the amino acid sequence AASYALIDMSSAYDY (SEQ ID NO:82);
wherein the CDR1 sequence comprises the amino acid sequence GRTFSRIA (SEQ ID NO:84); wherein the CDR2 comprises the amino acid sequence ISGNGGT (SEQ ID NO:85); and wherein the CDR3 comprises the amino acid sequence AADPNYRATYFPYGMDY (SEQ ID NO:86);
wherein the CDR1 sequence comprises the amino acid sequence GSIFSINA (SEQ ID NO:88); wherein the CDR2 comprises the amino acid sequence ITSGGTT (SEQ ID NO:89); and wherein the CDR3 comprises the amino acid sequence NLPWTSDLGWAVKDY (SEQ ID NO:90);
wherein the CDR1 sequence comprises the amino acid sequence GRASSTYV (SEQ ID NO:92); wherein the CDR2 comprises the amino acid sequence DTWGGAGT (SEQ ID NO:93); and wherein the CDR3 comprises the amino acid sequence AAGQGRSVTLFQPSTYDY (SEQ ID NO:94);
wherein the CDR1 sequence comprises the amino acid sequence TSINIYP (SEQ ID NO:96); wherein the CDR2 comprises the amino acid sequence VNRDGNT (SEQ ID NO:97); and the CDR3 sequence of the variable domain comprises the amino acid sequence NNFGSSS (SEQ ID NO:98);
wherein the CDR1 sequence comprises the amino acid sequence RSIDIYVA (SEQ ID NO:100); wherein the CDR2 comprises the amino acid sequence IHRGDTT (SEQ ID NO:101); and wherein the CDR3 comprises the amino acid sequence NDFGGTR (SEQ ID NO:102);
wherein the CDR1 sequence comprises the amino acid sequence GSIYGMMMMA (SEQ ID NO:104); wherein the CDR2 comprises the amino acid sequence FTRDGSTNY (SEQ ID NO:105); and wherein the CDR3 comprises the amino acid sequence NIQRY (SEQ ID NO:106);
wherein the CDR1 sequence comprises the amino acid sequence GFYFPNYA (SEQ ID NO:108); wherein the CDR2 comprises the amino acid sequence ITSAGGST (SEQ ID NO:109); and wherein the CDR3 comprises the amino acid sequence NADPSYGTRY (SEQ ID NO:110);
wherein the CDR1 sequence comprises the amino acid sequence GFTRKHYT (SEQ ID NO:112); wherein the CDR2 comprises the amino acid sequence ITTPDNST (SEQ ID NO:113); and wherein the CDR3 comprises the amino acid sequence GASALGGSSCAQSSSVLHRLFQ (SEQ ID NO:114);
wherein the CDR1 sequence comprises the amino acid sequence GSLPSDYV (SEQ ID NO:116); wherein the CDR2 comprises the amino acid sequence ITTADIT (SEQ ID NO:117); and wherein the CDR3 comprises the amino acid sequence KITILPSVSVY (SEQ ID NO:118);
wherein the CDR1 sequence comprises the amino acid sequence GFSLDYLA (SEQ ID NO:120); wherein the CDR2 comprises the amino acid sequence IRSSDGTI (SEQ ID NO:121); and wherein the CDR3 comprises the amino acid sequence GIQAGGSTGDIRLACGGMDH (SEQ ID NO:122);
wherein the CDR1 sequence comprises the amino acid sequence GFTFSHAV (SEQ ID NO:124); wherein the CDR2 comprises the amino acid sequence WRSAGGIT (SEQ ID NO:125); and wherein the CDR3 comprises the amino acid sequence KAFVVGSAY (SEQ ID NO:126);
wherein the CDR1 sequence comprises the amino acid sequence GTVFKIYV (SEQ ID NO:128); CDR2 sequence of the variable domain comprises the amino acid sequence ISNGGTP (SEQ ID NO:129); and t wherein the CDR3 comprises the amino acid sequence NRRQLEGRQSEDY (SEQ ID NO:130);
wherein the CDR1 sequence comprises the amino acid sequence RSINIYVA (SEQ ID NO:132); wherein the CDR2 comprises the amino acid sequence AHKDGGT (SEQ ID NO:133); and wherein the CDR3 comprises the amino acid sequence NAFGSSA (SEQ ID NO:134);
wherein the CDR1 sequence comprises the amino acid sequence GLSLSTDV (SEQ ID NO:136); wherein the CDR2 comprises the amino acid sequence IRSAGWIT (SEQ ID NO:137); and wherein the CDR3 comprises the amino acid sequence KVLRLPDGLAF (SEQ ID NO:138);
wherein the CDR1 sequence comprises the amino acid sequence GFTFSGYG (SEQ ID NO:140); wherein the CDR2 comprises the amino acid sequence STADSTP (SEQ ID NO:141); and wherein the CDR3 comprises the amino acid sequence RTRTAWEEY (SEQ ID NO:142);
wherein the CDR1 sequence comprises the amino acid sequence GSLRSGYV (SEQ ID NO:144); wherein the CDR2 comprises the amino acid sequence ITTGDIT (SEQ ID NO:145); and wherein the CDR3 comprises the amino acid sequence KITELPTVSVY (SEQ ID NO:146);
wherein the CDR1 sequence comprises the amino acid sequence GLTSGTYV (SEQ ID NO:148); wherein the CDR2 comprises the amino acid sequence IRDAGGIR (SEQ ID NO:149); and wherein the CDR3 comprises the amino acid sequence KFLRLPESLAY (SEQ ID NO:150);
wherein the CDR1 sequence comprises the amino acid sequence GSIFGVNT (SEQ ID NO:152); wherein the CDR2 comprises the amino acid sequence ISPGGYT (SEQ ID NO:153); and wherein the CDR3 comprises the amino acid sequence NYRSGTSRPNTN (SEQ ID NO:154); or
wherein the CDR1 sequence comprises the amino acid sequence GFTFSNYV (SEQ ID NO:156); wherein the CDR2 comprises the amino acid sequence VSESGQSR (SEQ ID NO:157); and wherein the CDR3 comprises the amino acid sequence sequence NTGSRTYGARY (SEQ ID NO:158).

2. The composition of claim 1, wherein the nanobody comprises the amino acid sequence selected from the group consisting of SEQ ID NO:4, SEQ ID NO:8, SEQ ID NO:12, SEQ ID NO:16, SEQ ID NO:20, SEQ ID NO:160, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:4, SEQ ID NO:8, SEQ ID NO:12, SEQ ID NO:16, SEQ ID NO:20, SEQ ID NO:160, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, or SEQ ID NO:159.

3. The composition of claim 1, wherein the nanobody is conjugated to a detectable moiety.

4. A method for treating reducing the risk of Clostridium difficile infection or reducing the severity of Clostridium difficile infection in a subject, comprising administering to the subject an effective amount of the composition of claim 1.

5. The method of claim 4, further comprising administering to the subject an antibiotic, an immunotherapeutic agent, an herbal remedy, a probiotic, or combinations thereof.

6. The method of claim 4, wherein the subject has previously suffered a Clostridium difficile infection.

7. A method for diagnosing Clostridium difficile infection in a subject, comprising contacting a feces sample from the subject with the composition of claim 1, and assaying the sample for binding of the nanobody to an antigen.

8. The method of claim 7, wherein the assay comprises an Enzyme-linked immunosorbent assay (ELISA) immunoassay.

9. The method of claim 7, wherein the method determines that the subject has a Clostridium difficile infection.

10. The method of claim 7, further comprising administering to the subject an effective amount of the composition of claim 1.


SEQUENCE LISTING (212 sequences)

SEQ ID NO: 1 - protein, 8 aa, synthetic construct
GRIFSIKS

SEQ ID NO: 2 - protein, 7 aa, synthetic construct
ITSGGST

SEQ ID NO: 3 - protein, 16 aa, synthetic construct
RRVVVTPYPDEYEYDY

SEQ ID NO: 4 - protein, 122 aa, synthetic construct
QVQLQESGGGLVQPGGSLRLSCAASGRIFSIKSMGWYRQAPGKQRELVADITSGGSTNYADSVKGRFTISRDSAWNTVYLQMNSLKPEDTAVYYCRRVVVTPYPDEYEYDYWGQGTQVTVSS

SEQ ID NO: 5 - protein, 8 aa, synthetic construct
GFIDDDYA

SEQ ID NO: 6 - protein, 8 aa, synthetic construct
ISSSNGKI

SEQ ID NO: 7 - protein, 20 aa, synthetic construct
AAETRGWSYCSGYGWSRYKY

SEQ ID NO: 8 - protein, 127 aa, synthetic construct
QVQLQESGGGSVQAGGSLRLSCAASGFIDDDYAIGWFRQAPGKEREGISCISSSNGKIHYADSVKGRFTISEDIAKKTVYLQMNFLKPEDTAVYYCAAETRGWSYCSGYGWSRYKYWGQGTQVTVSS

SEQ ID NO: 9 - protein, 8 aa, synthetic construct
GGTFSSYS

SEQ ID NO: 10 - protein, 7 aa, synthetic construct
ITWRGIT

SEQ ID NO: 11 - protein, 15 aa, synthetic construct
AARDRRAARIQEFDY

SEQ ID NO: 12 - protein, 121 aa, synthetic construct
QVQLQESGGGLVQAGGSLRLSCAASGGTFSSYSVGWFRQAPGLEREFVGMITWRGITYFEDFVKDRFNISRDNAKNTVYLQMNSLKPEDTAVYSCAARDRRAARIQEFDYWGQGTQVTVSS

SEQ ID NO: 13 - protein, 8 aa, synthetic construct
GRSFSINT

SEQ ID NO: 14 - protein, 7 aa, synthetic construct
ITTGGNT

SEQ ID NO: 15 - protein, 16 aa, synthetic construct
RTVVVTPYPDEFEYDY

SEQ ID NO: 16 - protein, 122 aa, synthetic construct
QVQLQESGGGLVQPGGSLRLSCAASGRSFSINTMGWYRQAPGNKRDMVATITTGGNTNYADSVKGRFTISRDNAKNTVYLQMNSLKPEDTAVYYCRTVVVTPYPDEFEYDYWGQGTQVTVSS

SEQ ID NO: 17 - protein, 8 aa, synthetic construct
GRSFSINT

SEQ ID NO: 18 - protein, 7 aa, synthetic construct
ITTGGNT

SEQ ID NO: 19 - protein, 16 aa, synthetic construct
RTVVVTPYPDEFEYDY

SEQ ID NO: 20 - protein, 126 aa, synthetic construct
QVQLQESGGGLVQAGDSLRLSCAASGRTFSRYAMGWFRQAPGKEREFVAAISWSGGTTYYADSVKGRFTISRDNAKNTVYLQMNSLKPEDTAVYYCAAGLFRGDLTRFKLDEYDYRGQGTQVTVSS

SEQ ID NO: 21 - protein, 8 aa, synthetic construct
GRTFSTHT

SEQ ID NO: 22 - protein, 8 aa, synthetic construct
IRWSDGMT

SEQ ID NO: 23 - protein, 11 aa, synthetic construct
GAGPTMYHPTY

SEQ ID NO: 24 - protein, 8 aa, synthetic construct
GGTFSRYA

SEQ ID NO: 25 - protein, 8 aa, synthetic construct
ISYSGATT

SEQ ID NO: 26 - protein, 19 aa, synthetic construct
AAGFFRGDLTKFKLDEYDY

SEQ ID NO: 27 - protein, 126 aa, synthetic construct
QVQLQESGGGLVQAGGSLRLSCVASGGTFSRYALGWFRQAPGKEREFVAAISYSGATTYYADSVKGRFTISRDNAKNTVFLQMNSLKPEDTAVYYCAAGFFRGDLTKFKLDEYDYRGQGTQVTVSS

SEQ ID NO: 28 - protein, 8 aa, synthetic construct
GRTFTTYN

SEQ ID NO: 29 - protein, 8 aa, synthetic construct
ITGLTRHT

SEQ ID NO: 30 - protein, 16 aa, synthetic construct
AVSSGGDLNERVNYEY

SEQ ID NO: 31 - protein, 123 aa, synthetic construct
QVQLQESGGGLVQAGGSLRLSCTASGRTFTTYNWAWFCQAPGKEREFVAAITGLTRHTYYADSVKGRFTISRDNAKNTVYLQMNSLKPEDTAVYFCAVSSGGDLNERVNYEYWGQGTQVTVSS

SEQ ID NO: 32 - protein, 8 aa, synthetic construct
GRTLNSYA

SEQ ID NO: 33 - protein, 8 aa, synthetic construct
ISRAGGMT

SEQ ID NO: 34 - protein, 15 aa, synthetic construct
AASFALVDSAGAYDY

SEQ ID NO: 35 - protein, 122 aa, synthetic construct
QVQLQESGGGLVQAGGSLRLSCAVSGRTLNSYAMGWFRQALGKEREFVAGISRAGGMTRYTDSVKGRFTISRDDAKNTVYLQMNSLKPDDTAVYSCAASFALVDSAGAYDYWGQGTQVTVSS

SEQ ID NO: 36 - protein, 8 aa, synthetic construct
GRAFSSYA

SEQ ID NO: 37 - protein, 8 aa, synthetic construct
ISWSGGST

SEQ ID NO: 38 - protein, 18 aa, synthetic construct
AADFSQPLLATVPDDYDY

SEQ ID NO: 39 - protein, 125 aa, synthetic construct
QVQLQESGGGLVQPGGSLRLSCAASGRAFSSYAMGWFRQTPGKEREFVAVISWSGGSTYYADSVKGRFTISRDNTKNMVYLQMMSLKPEDTAVYYCAADFSQPLLATVPDDYDYWGQGTQVTVSS

SEQ ID NO: 40 - protein, 8 aa, synthetic construct
GGTFSKTS

SEQ ID NO: 41 - protein, 7 aa, synthetic construct
ITWSGNT

SEQ ID NO: 42 - protein, 15 aa, synthetic construct
AARERTAARIQEFDY

SEQ ID NO: 43 - protein, 121 aa, synthetic construct
QVQLQESGGGLVQAGGSLRLSCAASGGTFSKTSVGWFRQAPGLEREFVALITWSGNTYFVDSVRERFAISRDNAENTVYLQMNSLEPEDTAVYYCAARERTAARIQEFDYWGQGTQVTVSS

SEQ ID NO: 44 - protein, 8 aa, synthetic construct
GSIFSINA

SEQ ID NO: 45 - protein, 7 aa, synthetic construct
ITSGGST

SEQ ID NO: 46 - protein, 15 aa, synthetic construct
HVPWTDDFGWAVKDY

SEQ ID NO: 47 - protein, 121 aa, synthetic construct
QVQLQESGGGLVQPGGSLRLSCAASGSIFSINAMGWYRQAPGKQRELVASITSGGSTNYADSVKGRFTISRDGAKNTVYLQMNSLKPEDTAVYYCHVPWTDDFGWAVKDYWGQGTQVTVSS

SEQ ID NO: 48 - protein, 8 aa, synthetic construct
GRTFSRYE

SEQ ID NO: 49 - protein, 8 aa, synthetic construct
INRLGRST

SEQ ID NO: 50 - protein, 17 aa, synthetic construct
AAGVRLNLPQIPDVIDF

SEQ ID NO: 51 - protein, 124 aa, synthetic construct
QVQLQESGGGLVQAGGSLRLSCADSGRTFSRYEMGWSRQAPGKEREFVAVINRLGRSTYYADSVKGRFTISRDNAKNTVYLQMNSLKPEDTAVYYCAAGVRLNLPQIPDVIDFWGQGTQVTVSS

SEQ ID NO: 52 - protein, 8 aa, synthetic construct
GGTFSSYS

SEQ ID NO: 53 - protein, 7 aa, synthetic construct
ITWRNNT

SEQ ID NO: 54 - protein, 15 aa, synthetic construct
AARERRVARIQEFDY

SEQ ID NO: 55 - protein, 121 aa, synthetic construct
QVQLQESGGGLVQAGDSLRLSCTASGGTFSSYSIGWFRQAPGLEREFVALITWRNNTYLGDSVRERFAISRDNAKNTVYLQMNSLKPEDTAVYSCAARERRVARIQEFDYWGQGTQVTVSS

SEQ ID NO: 56 - protein, 8 aa, synthetic construct
GRDFSSGA

SEQ ID NO: 57 - protein, 8 aa, synthetic construct
VGWSGGLI

SEQ ID NO: 58 - protein, 22 aa, synthetic construct
AVNSANSCAGYDCHDKPQTYNY

SEQ ID NO: 59 - protein, 128 aa, synthetic construct
QVQLQESGGGTVQPGGSLRLSCAASGRDFSSGAMGWFRQTPGNEREFVGVGWSGGLIDYSDSVKGRFTITRDKLKNEVYLRMDRLKPEDTAVYFCAVNSANSCAGYDCHDKPQTYNYWGQGTQVTVSS

SEQ ID NO: 60 - protein, 8 aa, synthetic construct
ERTFSAYT

SEQ ID NO: 61 - protein, 10 aa, synthetic construct
IKWSGSGGIT

SEQ ID NO: 62 - protein, 11 aa, synthetic construct
TAGPTVYNPHY

SEQ ID NO: 63 - protein, 120 aa, synthetic construct
QVQLQESGGGLVQAGGSLRLSCTPSERTFSAYTMAWYRQPPGKEREFAAGIKWSGSGGITLYADSVKGRFTISGDNAKSTVYLEMNSLKPEDTAVYYCTAGPTVYNPHYWGQGTQVTVSS

SEQ ID NO: 64 - protein, 8 aa, synthetic construct
GRTFSRYA

SEQ ID NO: 65 - protein, 8 aa, synthetic construct
ISYSGGTT

SEQ ID NO: 66 - protein, 19 aa, synthetic construct
AAGLFRGDLTRFTLDEYDY

SEQ ID NO: 67 - protein, 126 aa, synthetic construct
QVQLQESGGGLVQTGGSLRLSCTASGRTFSRYAMGWFRQASGKEREFVAAISYSGGTTYYSDSVKGRFTISRDNAKSTVSLQMNSLKPEDTAVYYCAAGLFRGDLTRFTLDEYDYRGQGTQVTVSS

SEQ ID NO: 68 - protein, 8 aa, synthetic construct
GRSFSSGA

SEQ ID NO: 69 - protein, 8 aa, synthetic construct
VGWSGGLI

SEQ ID NO: 70 - protein, 22 aa, synthetic construct
AINSANSCAGYDCHDKPQVYDY

SEQ ID NO: 71 - protein, 128 aa, synthetic construct
QVQLQESGGGLVQPGGSLRLSCAASGRSFSSGAMGWFRQAPGKEREFVGVGWSGGLIDYAASVRGRFTITRDKAKNTVYLRMDSLRPEDTAVYYCAINSANSCAGYDCHDKPQVYDYWGQGTQVTVSS

SEQ ID NO: 72 - protein, 8 aa, synthetic construct
GRTFSRYA

SEQ ID NO: 73 - protein, 8 aa, synthetic construct
ISWSGDTT

SEQ ID NO: 74 - protein, 19 aa, synthetic construct
AAGLFRGDLTKFELDEYDY

SEQ ID NO: 75 - protein, 126 aa, synthetic construct
QVQLQESGGGLVQAGGSLRLSCAASGRTFSRYAMGWFRQAPGKEREFVAAISWSGDTTYDADSVKGRFTVSRDNAKNTVYLQMNSLKPEDTAVYYCAAGLFRGDLTKFELDEYDYRGQGTQVTVSS

SEQ ID NO: 76 - protein, 8 aa, synthetic construct
GHSFSTSA

SEQ ID NO: 77 - protein, 8 aa, synthetic construct
ISWAGGKI

SEQ ID NO: 78 - protein, 22 aa, synthetic construct
AANSQNMCSGWDCEKQPRVYDF

SEQ ID NO: 79 - protein, 128 aa, synthetic construct
QVQLQESGGGLVQPGGSLTLSCIASGHSFSTSAMAWFRQAPGKEREVVGISWAGGKIDYADFVRGRFTISRDNAKNTVSLQMNGLKPEETAVYYCAANSQNMCSGWDCEKQPRVYDFWGQGTQVTVSS

SEQ ID NO: 80 - protein, 8 aa, synthetic construct
GRTLSSYA

SEQ ID NO: 81 - protein, 8 aa, synthetic construct
ISRGGGMT

SEQ ID NO: 82 - protein, 15 aa, synthetic construct
AASYALIDMSSAYDY

SEQ ID NO: 83 - protein, 122 aa, synthetic construct
QVQLQESGGGLVQAGGSLRLSCAVSGRTLSSYAMGWFRQALGKEREFVAGISRGGGMTRYTDSVKGRFTISRDDAKNTVYLQMNSLKPDDTAVYSCAASYALIDMSSAYDYWGQGTQVTVSS

SEQ ID NO: 84 - protein, 8 aa, synthetic construct
GRTFSRIA

SEQ ID NO: 85 - protein, 7 aa, synthetic construct
ISGNGGT

SEQ ID NO: 86 - protein, 17 aa, synthetic construct
AADPNYRATYFPYGMDY

SEQ ID NO: 87 - protein, 123 aa, synthetic construct
QVQLQESGGGLVQAGGSLRLSCAASGRTFSRIAMGWFRQVPGKEREFVAGISGNGGTFYTDSVKGRFTISRDNTKNTVYLQMNSLRPEDTAVYYCAADPNYRATYFPYGMDYYGRGTQVTVSS

SEQ ID NO: 88 - protein, 8 aa, synthetic construct
GSIFSINA

SEQ ID NO: 89 - protein, 7 aa, synthetic construct
ITSGGTT

SEQ ID NO: 90 - protein, 15 aa, synthetic construct
NLPWTSDLGWAVKDY

SEQ ID NO: 91 - protein, 121 aa, synthetic construct
QVQLQESGGGLVQPGGSLRLSCAASGSIFSINAMGWYRQAPGKQRELVAKITSGGTTNYADSVKGRFTISRDGAKNTVYLQMNSLKPEDTAVYYCNLPWTSDLGWAVKDYWGQGTQVTVSS

SEQ ID NO: 92 - protein, 8 aa, synthetic construct
GRASSTYV

SEQ ID NO: 93 - protein, 8 aa, synthetic construct
DTWGGAGT

SEQ ID NO: 94 - protein, 18 aa, synthetic construct
AAGQGRSVTLFQPSTYDY

SEQ ID NO: 95 - protein, 125 aa, synthetic construct
QVQLQESGGGLVQAGGSLRLSCAASGRASSTYVMAWFRQAPGKEREFVAADTWGGAGTYYAPSVKGRFTISRDNAKNMLYLQMNSLKPEDTAAYYCAAGQGRSVTLFQPSTYDYWGQGTQVTVSS

SEQ ID NO: 96 - protein, 7 aa, synthetic construct
TSINIYP

SEQ ID NO: 97 - protein, 7 aa, synthetic construct
VNRDGNT

SEQ ID NO: 98 - protein, 7 aa, synthetic construct
NNFGSSS

SEQ ID NO: 99 - protein, 113 aa, synthetic construct
QVQLQESGGGLVQPGGSLRLTCTSSTSINIYPYMGWYRQAPGKQRERVATVNRDGNTNYLDSVKGRFTITRDDAKKTICLQMNNLEPEDTAVYYCNNFGSSSWGQGTQVTVSS

SEQ ID NO: 100 - protein, 8 aa, synthetic construct
RSIDIYVA

SEQ ID NO: 101 - protein, 7 aa, synthetic construct
IHRGDTT

SEQ ID NO: 102 - protein, 7 aa, synthetic construct
NDFGGTR

SEQ ID NO: 103 - protein, 113 aa, synthetic construct
QVQLQESGGALVQPGGSLRLTCTSSRSIDIYVAMAWYRQAPGKQRERVATIHRGDTTNYSDSVKGRFTISRDNAKNTITLQMNNLKPEDTAVYYCNDFGGTRWGQGTQVTVSS

SEQ ID NO: 104 - protein, 10 aa, synthetic construct
GSIYGMMMMA

SEQ ID NO: 105 - protein, 9 aa, synthetic construct
FTRDGSTNY

SEQ ID NO: 106 - protein, 5 aa, synthetic construct
NIQRY

SEQ ID NO: 107 - protein, 111 aa, synthetic construct
QVQLQESGGGSVQPGGSLRLSCAASGSIYGMMMMAWYRQRTGEQRELVANFTRDGSTNYVDSVKGRFTISRDNDKKMVYLQMKDLKPEDTAVYYCNIQRYWGKGTAVTVSS

SEQ ID NO: 108 - protein, 8 aa, synthetic construct
GFYFPNYA

SEQ ID NO: 109 - protein, 8 aa, synthetic construct
ITSAGGST

SEQ ID NO: 110 - protein, 10 aa, synthetic construct
NADPSYGTRY

SEQ ID NO: 111 - protein, 117 aa, synthetic construct
QVQLQESGGGLVQPGGSLRLSCATSGFYFPNYAMSWHRQAPGQERELVARITSAGGSTDYADSVKGRFTISRDNSKNTVYLQMNGLISEDTAVYYCNADPSYGTRYWGQGTQVTVSS

SEQ ID NO: 112 - protein, 8 aa, synthetic construct
GFTRKHYT

SEQ ID NO: 113 - protein, 8 aa, synthetic construct
ITTPDNST

SEQ ID NO: 114 - protein, 22 aa, synthetic construct
GASALGGSSCAQSSSVLHRLFQ

SEQ ID NO: 115 - protein, 129 aa, synthetic construct
QVQLQESGGGLVQPGGSLRLSCTASGFTRKHYTIGWFRQAPGKEREGVSCITTPDNSTYYKDSVKGRFTISRDNAKNTVYLQMNNVKPEDTAVYYCGASALGGSSCAQSSSVLHRLFQWGQGTQVTVSS

SEQ ID NO: 116 - protein, 8 aa, synthetic construct
GSLPSDYV

SEQ ID NO: 117 - protein, 7 aa, synthetic construct
ITTADIT

SEQ ID NO: 118 - protein, 11 aa, synthetic construct
KITILPSVSVY

SEQ ID NO: 119 - protein, 117 aa, synthetic construct
QVQLQESGGGLVQPGGSLRLSCAASGSLPSDYVTSWHRQAPGKQRELVASITTADITNYAASVKGRFTISRDRAKNMGYLQMNSLLPEDTAVYYCKITILPSVSVYWGQGTQVTVSS

SEQ ID NO: 120 - protein, 8 aa, synthetic construct
GFSLDYLA

SEQ ID NO: 121 - protein, 8 aa, synthetic construct
IRSSDGTI

SEQ ID NO: 122 - protein, 20 aa, synthetic construct
GIQAGGSTGDIRLACGGMDH

SEQ ID NO: 123 - protein, 127 aa, synthetic construct
QVQLQESGGGLVQPGGSLRLSCAASGFSLDYLAIGWFRQAPGKGREGVSCIRSSDGTIFYSDSVKGRFTMSRDNAKNTVYLQMNSLKPEDTAVYHCGIQAGGSTGDIRLACGGMDHWGNGTQVTVSS

SEQ ID NO: 124 - protein, 8 aa, synthetic construct
GFTFSHAV

SEQ ID NO: 125 - protein, 8 aa, synthetic construct
WRSAGGIT

SEQ ID NO: 126 - protein, 9 aa, synthetic construct
KAFVVGSAY

SEQ ID NO: 127 - protein, 116 aa, synthetic construct
QVQLQESGGGLAQPGGSLRLSCAASGFTFSHAVMSWFRQAPGKERELVAGWRSAGGITNYADSVKGRFTISGDNAKDVVYLQMNSLKPEDTAVYYCKAFVVGSAYWGQGTQVTVSS

SEQ ID NO: 128 - protein, 8 aa, synthetic construct
GTVFKIYV

SEQ ID NO: 129 - protein, 7 aa, synthetic construct
ISNGGTP

SEQ ID NO: 130 - protein, 13 aa, synthetic construct
NRRQLEGRQSEDY

SEQ ID NO: 131 - protein, 119 aa, synthetic construct
QVQLQESGGGLVLPGGSLRLSCAASGTVFKIYVMGWYRQAPGKQRELVATISNGGTPNYADSVKGRFTISGDRAKNTVFLQMSSLNVEDTAVYYCNRRQLEGRQSEDYWGQGTQVTVSS

SEQ ID NO: 132 - protein, 8 aa, synthetic construct
RSINIYVA

SEQ ID NO: 133 - protein, 7 aa, synthetic construct
AHKDGGT

SEQ ID NO: 134 - protein, 7 aa, synthetic construct
NAFGSSA

SEQ ID NO: 135 - protein, 113 aa, synthetic construct
QVQLQESGGGVVQPGGSLRLTCTSSRSINIYVAMGWYRQAPGKQRERVATAHKDGGTRYSDSVKGRFTISRDDDKNTVYLQMNNLEPGDTAVYYCNAFGSSAWGQGTQVTVSS

SEQ ID NO: 136 - protein, 8 aa, synthetic construct
GLSLSTDV

SEQ ID NO: 137 - protein, 8 aa, synthetic construct
IRSAGWIT

SEQ ID NO: 138 - protein, 11 aa, synthetic construct
KVLRLPDGLAF

SEQ ID NO: 139 - protein, 118 aa, synthetic construct
QVQLQESGGGLVQPGGSLRLSCLASGLSLSTDVMSWFRQAPGKERELVAGIRSAGWITNYADSVKGRFTISVDSAKNTVYLQMNSLKPEDTAVYYCKVLRLPDGLAFWGQGTQVTVSS

SEQ ID NO: 140 - protein, 8 aa, synthetic construct
GFTFSGYG

SEQ ID NO: 141 - protein, 7 aa, synthetic construct
STADSTP

SEQ ID NO: 142 - protein, 9 aa, synthetic construct
RTRTAWEEY

SEQ ID NO: 143 - protein, 115 aa, synthetic construct
QVQLQESGGGLVQPGGFLRLACAASGFTFSGYGVSWYRQAPGKEREFIAASTADSTPNYAGSVKGRFTISRDSAKNMVYLQMNNLKSEDTGVYYCRTRTAWEEYWGQGTQVTVSS

SEQ ID NO: 144 - protein, 8 aa, synthetic construct
GSLRSGYV

SEQ ID NO: 145 - protein, 7 aa, synthetic construct
ITTGDIT

SEQ ID NO: 146 - protein, 11 aa, synthetic construct
KITELPTVSVY

SEQ ID NO: 147 - protein, 117 aa, synthetic construct
QVQLQESGGGLVQPGGSLRLSCAASGSLRSGYVMSWHRQAPGKQRELVASITTGDITQYGASVKGRFTISRDETKNMGWLQMNSLLPEDTAVYYCKITELPTVSVYWGQGTQVTVSS

SEQ ID NO: 148 - protein, 8 aa, synthetic construct
GLTSGTYV

SEQ ID NO: 149 - protein, 8 aa, synthetic construct
IRDAGGIR

SEQ ID NO: 150 - protein, 11 aa, synthetic construct
KFLRLPESLAY

SEQ ID NO: 151 - protein, 118 aa, synthetic construct
QVQLQESGGGLVQPGGSLRLSCAASGLTSGTYVMSWFRQAPGKEREFVAAIRDAGGIRNYADSVKGRFTISRDAAKNMIFLQMNSLKPEDTAVYYCKFLRLPESLAYWGQGTQVTVSS

SEQ ID NO: 152 - protein, 8 aa, synthetic construct
GSIFGVNT

SEQ ID NO: 153 - protein, 7 aa, synthetic construct
ISPGGYT

SEQ ID NO: 154 - protein, 12 aa, synthetic construct
NYRSGTSRPNTN

SEQ ID NO: 155 - protein, 118 aa, synthetic construct
QVQLQESGGGLVHVGESLTLSCVAPGSIFGVNTMAWYRQAPGKQRELVAAISPGGYTNYAEFVKGRFGISRDKAKNTVYLQMNDLKPEDTAVYYCNYRSGTSRPNTNWGQGTQVTVSS

SEQ ID NO: 156 - protein, 8 aa, synthetic construct
GFTFSNYV

SEQ ID NO: 157 - protein, 8 aa, synthetic construct
VSESGQSR

SEQ ID NO: 158 - protein, 11 aa, synthetic construct
NTGSRTYGARY

SEQ ID NO: 159 - protein, 118 aa, synthetic construct
QVQLQESGGGLVQAGGSLTLSCTASGFTFSNYVMSWYRQAPGKEREWVASVSESGQSRTYADSVKGRFTISRDNTNFSVYLQMNNLNSEDTAVYYCNTGSRTYGARYWGQGTQVTVSS

SEQ ID NO: 160 - protein, 118 aa, synthetic construct
QVQLQESGGGLVQAGGSLRLSCAASGRTFSTHTMAWFRQAPGKEREFVTGIRWSDGMTIYADSVKGRFTISRDKATNTMYLEMNTLKPDDTAVYYCGAGPTMYHPTYWGQGTQVTVSS

SEQ ID NO: 161 - protein, 8 aa, synthetic construct
IRRGISRK

SEQ ID NO: 162 - protein, 6 aa, synthetic construct
KRKRAV

SEQ ID NO: 163 - protein, 9 aa, synthetic construct
PKPKRQTKR

SEQ ID NO: 164 - protein, 8 aa, synthetic construct
RRRRHCNR

SEQ ID NO: 165 - protein, 8 aa, synthetic construct
IPDPTGQS

SEQ ID NO: 166 - protein, 8 aa, synthetic construct
IKREREND

SEQ ID NO: 167 - protein, 17 aa, synthetic construct
KKLQEQEKQQKVEFRKR

SEQ ID NO: 168 - protein, 9 aa, synthetic construct
GPNKKKRKL

SEQ ID NO: 169 - protein, 19 aa, synthetic construct
RRRRASAPISQWSSSRRSR

SEQ ID NO: 170 - protein, 18 aa, synthetic construct
KLALKLALKALKAALKLA

SEQ ID NO: 171 - protein, 13 aa, synthetic construct
FLPLIGRVLSGIL

SEQ ID NO: 172 - protein, 26 aa, synthetic construct
CVQWSLLRGYQPCCVQWSLLRGYQPC

SEQ ID NO: 173 - protein, 32 aa, synthetic construct
RKKRKGSGSRKKRKGSGSRKKRKGSGSRKKRK

SEQ ID NO: 174 - protein, 30 aa, synthetic construct
CVQWSLLRGYQPCGSGSCVQWSLLRGYQPC

SEQ ID NO: 175 - protein, 32 aa, synthetic construct
KKRRRGSGSKKRRRGSGSKKRRRGSGSKKRRR

SEQ ID NO: 176 - protein, 32 aa, synthetic construct
RKKRRGSGSRKKRRGSGSRKKRRGSGSRKKRR

SEQ ID NO: 177 - protein, 17 aa, synthetic construct
RQIKIWFQNRRMKWKKC

SEQ ID NO: 178 - protein, 28 aa, synthetic construct
GRKRKRSGRKRKRSGRKRKRSGRKRKRS

SEQ ID NO: 179 - protein, 28 aa, synthetic construct
PRKKRGRPRKKRGRPRKKRGRPRKKRGR

SEQ ID NO: 180 - protein, 13 aa, synthetic construct
CVQWSLLRGYQPC

SEQ ID NO: 181 - protein, 9 aa, synthetic construct
  VARIANT 1: note = X is Arg or Lys
  VARIANT 3: note = X is Arg or Lys
  VARIANT 5: note = X is Arg or Lys
XKXRXGSGS

SEQ ID NO: 182 - protein, 6 aa, synthetic construct
HHHHHH

SEQ ID NO: 183 - protein, 9 aa, synthetic construct
RKKRRQRRR

SEQ ID NO: 184 - protein, 12 aa, synthetic construct
RQARRNRRRRWR

SEQ ID NO: 185 - protein, 7 aa, synthetic construct
PKKKRKV

SEQ ID NO: 186 - protein, 12 aa, synthetic construct
HHHHHHHHHHHH

SEQ ID NO: 187 - protein, 7 aa, synthetic construct
  MOD_RES 2: note = 2-naphthylalanine
FARRRRQ

SEQ ID NO: 188 - protein, 7 aa, synthetic construct
  MOD_RES 2: note = 2-naphthylalanine
  SITE 1: note = D-phenylalanine
  SITE 4: note = D-arginine
  SITE 6: note = D-arginine
FARRRRQ

SEQ ID NO: 189 - protein, 8 aa, synthetic construct
  MOD_RES 3: note = 2-naphthylalanine
  SITE 2: note = D-phenylalanine
  SITE 5: note = D-arginine
  SITE 7: note = D-arginine
FFARRRRQ

SEQ ID NO: 190 - protein, 7 aa, synthetic construct
  SITE 2: note = tert-butanoyl
FXRRRRQ

SEQ ID NO: 191 - protein, 7 aa, synthetic construct
  SITE 1: note = 2,3-diaminopropionic acid
  SITE 2: note = hexanoyl
XXRRRRQ

SEQ ID NO: 192 - protein, 7 aa, synthetic construct
  SITE 1: note = 2,3-diaminopropionic acid
  SITE 2: note = octanoyl
XXRRRRQ

SEQ ID NO: 193 - protein, 7 aa, synthetic construct
  SITE 1: note = 2,3-diaminopropionic acid
  SITE 2: note = decanoyl
XXRRRRQ

SEQ ID NO: 194 - protein, 7 aa, synthetic construct
  SITE 1: note = 2,3-diaminopropionic acid
  SITE 2: note = 1-pyrenylbutanoyl
XXRRRRQ

SEQ ID NO: 195 - protein, 7 aa, synthetic construct
  SITE 1: note = 2,3-diaminopropionic acid
  SITE 2: note = 3,3-diphenoyl
XXRRRRQ

SEQ ID NO: 196 - protein, 7 aa, synthetic construct
  SITE 1: note = 2,3-diaminopropionic acid
  SITE 2: note = fluorenylmethyloxycarbonoyl
XXRRRRQ

SEQ ID NO: 197 - protein, 7 aa, synthetic construct
  SITE 1: note = 2,3-diaminopropionic acid
  SITE 2: note = pyrenol
XXRRRRQ

SEQ ID NO: 198 - protein, 7 aa, synthetic construct
  SITE 1: note = 2,3-diaminopropionic acid
  SITE 2: note = decanoyl
  SITE 4: note = D-arginine
  SITE 6: note = D-arginine
XXRRRRQ

SEQ ID NO: 199 - protein, 7 aa, synthetic construct
  SITE 1: note = 2,3-diaminopropionic acid
  SITE 2: note = decanoyl
XXRRRRQ

SEQ ID NO: 200 - protein, 7 aa, synthetic construct
  SITE 1: note = 2,3-diaminopropionic acid
  SITE 2: note = decanoyl
XXARRRQ

SEQ ID NO: 201 - protein, 7 aa, synthetic construct
  SITE 1: note = 2,3-diaminopropionic acid
  SITE 2: note = decanoyl
XXRRRAQ

SEQ ID NO: 202 - protein, 8 aa, synthetic construct
  SITE 1: note = 2,3-diaminopropionic acid
  SITE 2: note = decanoyl
XXRRRRRQ

SEQ ID NO: 203 - protein, 7 aa, synthetic construct
  SITE 2: note = decanoyl
KXRRRRQ

SEQ ID NO: 204 - protein, 6 aa, synthetic construct
  SITE 1: note = 2,3-diaminopropionic acid
  SITE 2: note = decanoyl
XXRRRQ

SEQ ID NO: 205 - protein, 7 aa, synthetic construct
  SITE 1: note = ornithine
  SITE 2: note = decanoyl
XXRRRRQ

SEQ ID NO: 206 - protein, 7 aa, synthetic construct
  SITE 2: note = decanoyl
  SITE 4: note = D-arginine
  SITE 6: note = D-arginine
KXRRRRQ

SEQ ID NO: 207 - protein, 7 aa, synthetic construct
  SITE 2: note = decanoyl
  SITE 3: note = D-arginine
  SITE 5: note = D-arginine
KXRRRRQ

SEQ ID NO: 208 - protein, 7 aa, synthetic construct
  SITE 2: note = decynoyl
DXRRRRQ

SEQ ID NO: 209 - protein, 7 aa, synthetic construct
  SITE 2: note = decynoyl
  SITE 4: note = D-arginine
  SITE 6: note = D-arginine
DXRRRRQ

SEQ ID NO: 210 - protein, 7 aa, synthetic construct
  SITE 2: note = decynoyl
  SITE 4: note = D-arginine
  SITE 6: note = D-arginine
QXRRRRQ

SEQ ID NO: 211 - protein, 7 aa, synthetic construct
  SITE 1: note = decynoyl
  SITE 3: note = D-arginine
  SITE 5: note = D-arginine
DXRRRRQ

SEQ ID NO: 212 - protein, 7 aa, synthetic construct
  SITE 2: note = decynoyl
  SITE 3: note = D-arginine
  SITE 5: note = D-arginine
EXRRRRQ